SEQUENCE LISTING (11 sequences)

SEQ ID NO: 1 - DNA, 49 bp, Artificial Sequence
  SGR-beads-1 sequence
aatgatacggcgaccaccgagatctacactctttccctacacgacgctc

SEQ ID NO: 2 - DNA, 54 bp, Artificial Sequence
  SGR-tag-1 sequence
tggagttcagacgtgtgctcttccgatctgttgtcaagatgctaccgttcagag

SEQ ID NO: 3 - DNA, 25 bp, Artificial Sequence
  SGR-beads-2 sequence
aatgatacggcgaccaccgagatct

SEQ ID NO: 4 - DNA, 57 bp, Artificial Sequence
  SGR-tag-2 sequence
caagcagaagacggcatacgagattcgccttagtgactggagttcagacgtgtgctc

SEQ ID NO: 5 - protein, 124 aa, Artificial Sequence
  Engineered Neo2
MLVNRICGKGIDGGSPKKKIQLHAEHALYDALMILNIVKTNSPPAEEKLEDYAFNFELILEEIARLFESGDQKDEAEKAKRMKEWMKRIKTTASEDEQEEMANAIITILQSWIFSAVDHHHHHH

SEQ ID NO: 6 - protein, 143 aa, Artificial Sequence
  Engineered Nb-Pd-L1
MDQVQLQESGGGLVQPGGSLRLSCAASGKMSSRRCMAWFRQAPGKERERVAKLLTTSGSTYLADSVKGRFTISQNNAKSTVYLQMNSLKPEDTAMYYCAADSFEDPTCTLVTSSGAFQYWGQGTQVTVSSLPETGGCHHHHHH

SEQ ID NO: 7 - protein, 243 aa, Artificial Sequence
  Engineered GFP
MSKGEELFTGVVPILVELDGDVNGHKFSVRGEGEGDATNGKLTLKFICTTGKLPVPWPTLVTTLTYGVQCFSRYPDHMKRHDFFKSAMPEGYVQERTISFKDDGTYKTRAEVKFEGDTLVNRICLKGIDFKEDGNILGHKLEYNFNSHNVYITADKQKNGIKANFKIRHNVEDGSVQLADHYQQNTPIGDGPVLLPDNHYLSTQSVLSKDPNEKRDHMVLLEFVTAAGITHGGGGLEHHHHHH

SEQ ID NO: 8 - protein, 214 aa, Artificial Sequence
  Trastuzumab light chain
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 9 - protein, 453 aa, Artificial Sequence
  Trastuzumab heavy chain
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESLTQPEGKNNTTKPPKVSNKALPAPIEKTISKSRKQPREPQVYTLPPSREEMTKNQVSLTCLVKSLKSPK

SEQ ID NO: 10 - DNA, 20 bp, Artificial Sequence
  20ntssDNA
agcagcacagaggtcagatg

SEQ ID NO: 11 - DNA, 59 bp, Artificial Sequence
  59ntssDNA
tgtcaagatgctaccgttcagagcgcaagacactccacaaaaaaaaaaaaaaaaaaaaa

(19) United States
(12) Patent Application Publication
(10) Pub. No.: US 2024/0261425 A1
(43) Pub. Date: Aug. 8, 2024

(54) CONJUGATE AND THE PREPARING METHOD AND USE THEREOF

(71) Applicant: Nanjing University, Nanjing (CN)

(72) Inventors: Jie LI, Nanjing (CN); Qingsong WU, Nanjing (CN); Wanxing SHA, Nanjing (CN); Yang YANG, Nanjing (CN)

(73) Assignee: Nanjing University, Nanjing (CN)

(21) Appl. No.: 18/554,325

(22) PCT Filed: Apr. 8, 2022
(86) PCT No.: PCT/CN2022/085706

§ 371 Date: Oct. 6, 2023

(30) Foreign Application Priority Data
Apr. 9, 2021 (WO) PCT/CN2021/086094

(51) Int. Cl. A61K 47/68; A61K 47/54; A61P 35/00

(52) U.S. Cl. CPC A61K 47/6851; A61K 47/557; A61P 35/00

(57) ABSTRACT

A conjugate may have formula (1), i.e., M-[(L1)ₐ-(L2)_{b}-(D)_{c}] (1), wherein M is a biological macromolecule having a nucleophilic functional group, M is linked to L1 with the nucleophilic functional of M, D is a functional molecule, L2 is a linker, and L1 is a compound of formula 1

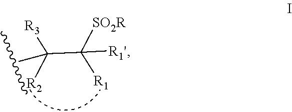

wherein R is —F or —OH, and L2 is linked to R₁, R₃ or R₂, a is an integer of 1 to 10, b, c is each independently an integer of 0 to 10, provided that b and c are not simultaneously 0. R₁, R₂, and R₃, may independently be H, optionally substituted alkyl, or optionally substituted aryl, and R₁, may be H or isotope thereof Δ C may link R₁ and a C linking R₂ form a ring.

### BACKGROUND OF THE INVENTION

The modification of a protein may give the protein new properties and functions. The common protein modification strategy is to modify the amino acid residues on the protein. Because lysine and cysteine have high affinity reactivity, the modification of the protein often occurs on these two amino acids.

In 2019, three antibody-conjugated drugs Fam-trastuzumab Deruxtecan (for treating HER2-positive unresectable or metastatic breast cancer), Polatuzumab Vedotin (for treating relapsed/refractory diffuse large B cells Lymphoma) and Enfortumab Vedotin (for treating locally advanced or metastatic urothelial cancer) were approved by the FDA, and are achieved by modifying the reduced interchain disulfide bonds on the antibody, and the linkers thereof are maleimide. It is well known that the adducts of maleimide and sulfhydryl are unstable and prone to thiol exchange in physiological condition. The ADC synthesized by the addition reaction between the maleimide and the sulfhydryl may cause the drug to be released in advance, which may seriously affect the stability and efficacy of the ADC.

Although chemists have achieved results in improving the stability of maleimide and seeking new stable linkers, the problems have not been overcome yet. Hence, a kind of linker satisfying the demand of preparing the conjugate, especially in balancing the activity and stability, is urgent to be developed.

### SUMMARY OF THE INVENTION

The present application provides a kind of potential linker which is promising in applying in preparing a conjugate, for example, an ADC. The linker may react with a nucleophilic functional group of a biological macromolecule (for example, a —SH of a cysteine) with relatively enhanced efficiency and/or chemoselectivity. In the present application, the linker and/or the conjugate comprising and/or being preparing through the linker may be stable in both in vitro and in vivo environment. For example, the conjugate (for example, an ADC) in the present application may exhibit more effective and/or more efficient killing of a target cell. For the conjugate (for example, an ADC) in the present application may be more stable and/or safer (particularly at a relatively higher concentration). In the present application, the balancing of the activity and stability may be solved by the conjugate of the present application.

In one aspect, the present application provides a conjugate of formula 1, M-[(L1)ₐ-(L2)_{b}-(D)_{c}]1, wherein L1 is a compound of formula 1,

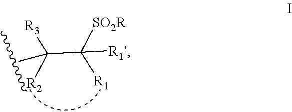

R is —F or —OH, wherein M is a biological macromolecule, and M is linked to L1 with a nucleophilic functional group of M, L2 is a linker, and L2 is linked to R₁, R₃ or R₂, D is a functional molecule, a is an integer of 1 to 10, b, c is each independently an integer of 0 to 10, provided that b and c are not simultaneously 0, wherein R₁ is H, an optionally substituted alkyl or an optionally substituted aryl, wherein R₁′ is H or the isotope thereof, wherein R₂ is H, an optionally substituted alkyl or an optionally substituted aryl, wherein R₃ is H, an optionally substituted alkyl or an optionally substituted aryl, optionally, a C linking R₁ and a C linking R₂ form a ring.

In some embodiments, said nucleophilic functional group of M is selected from a group consisting of —SH, —NH₂, —SeH, —OH, and

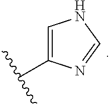 [SMILES: *c1c[nH]cn1]

In one aspect, the present application provides a conjugate of formula 2, M-S-[(L1)ₐ-(L2)_{b}-(D)_{c}] 2, wherein M-S is a biological macromolecule with a cysteine, M-S is linked to L1 with the cysteine, L1 is a compound of formula 1,

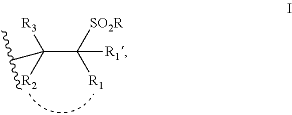

R is —F or —OH, L2 is a linker, L2 is linked to R₁, R₃ or R₂, D is a functional molecule, a is an integer of 1 to 10, b, c is each independently an integer of 0 to 10, provided that b and c are not simultaneously 0, wherein R₁ is H, an optionally substituted alkyl or an optionally substituted Aryl, wherein R₁′ is H or the isotope thereof, wherein R₂ is H, an optionally substituted alkyl or an optionally substituted Aryl, wherein R₃ is H, an optionally substituted alkyl or an optionally substituted Aryl, optionally, a C linking R₁ and a C linking R₂ form a ring.

In some embodiments, in the conjugate of formula 1 and/or the conjugate of formula 2, M is selected from a group consisting of a protein, a DNA, a RNA, and a virus.

In some embodiments, in the conjugate of formula 1 and/or the conjugate of formula 2, M is a biological macromolecule expressed on the surface of a cell.

In some embodiments, in the conjugate of formula 1 and/or the conjugate of formula 2, M is an antigen binding protein or a fragment thereof.

In some embodiments, in the conjugate of formula 1 and/or the conjugate of formula 2, M is a monoclonal antibody, a chimeric antibody, a humanized antibody, a human engineered antibody, a human antibody, a single chain antibody(scFv) or an antibody fragment.

In some embodiments, in the conjugate of formula 1 and/or the conjugate of formula 2, L2 is selected from the group consisting of a cleavable linker, a non-cleavable linker, a hydrophilic linker, a hydrophobic linker, a procharged linker, an uncharged linker and a dicarboxylic acid-based linker.

In some embodiments, in the conjugate of formula 1 and/or the conjugate of formula 2, L2 is selected from the group consisting of VC-PAB, N-succinimidyl-3-(2-pyridyldithio)propionate (SPDP), N-succinimidyl 4-(2-pyridyldithio)pentanoate (SPP), N-succinimidyl 4-(2-pyridyldithio)butanoate (SPDB), N-succinimidyl-4-(2-pyridyldithio)-2-sulfo-butanoate (sulfo-SPDB), N-succinimidyl iodoacetate (SIA), N-succinimidyl(4-iodoacetyl)aminobenzoate (SIAB), maleimide PEG NHS, N-succinimidyl 4-(maleimidomethyl) cyclohexanecarboxylate (SMCC), N-sulfosuccinimidyl 4-(maleimidomethyl) cyclohexanecarboxylate (sulfo-SMCC) or 2,5-dioxopyrrolidin-1-yl 17-(2,5-dioxo-2,5-dihydro-1H-pyrrol-1-yl)-5,8,11,14-tetraoxo-4,7,10,13-tetraazaheptadecan-1-oate (CX1-1).

In some embodiments, in the conjugate of formula 1 and/or the conjugate of formula 2, D has a biological function.

In some embodiments, in the conjugate of formula 1 and/or the conjugate of formula 2, D and/or a derivative thereof is capable of inhibiting the growth of a tumor cell.

In some embodiments, in the conjugate of formula 1 and/or the conjugate of formula 2, D is a drug.

In some embodiments, in the conjugate of formula 1 and/or the conjugate of formula 2, D is selected from the group consisting of a V-ATPase inhibitor, a pro-apoptotic agent, a Bcl2 inhibitor, an MCL1 inhibitor, a HSP90 inhibitor, an IAP inhibitor, an mTor inhibitor, a microtubule stabilizer, a microtubule destabilizer, an auristatin, a dolastatin, a maytansinoid, a MetAP (methionine aminopeptidase), an inhibitor of nuclear export of proteins CRM1, a DPPIV inhibitor, proteasome inhibitors, inhibitors of phosphoryl transfer reactions in mitochondria, a protein synthesis inhibitor, a kinase inhibitor, a CDK2 inhibitor, a CDK9 inhibitor, a kinesin inhibitor, an HDAC inhibitor, a DNA damaging agent, a DNA alkylating agent, a DNA intercalator, a DNA minor groove binder, a DHFR inhibitor, a nucleoside analog, a HD AC inhibitor; an anthracycline; a NAMPT inhibitor; a hydrophilic prodrug; a SN-38 glucuronide, a etoposide phosphate; a nitrogen mustard, a proteosome inhibitor, a cytokine, a Toll-like receptor agonist and a Sting agonist.

In some embodiments, in the conjugate of formula 1 and/or the conjugate of formula 2, D is MMAE or a derivative thereof; melphalan or a derivative thereof; lenalidomide or a derivative thereof; IL-2 or a derivative thereof; neoleukin-2/15 or a derivative thereof; T785 or a derivative thereof; or, MSA-2 or a derivative thereof.

In some embodiments, in the conjugate of formula 1 and/or the conjugate of formula 2, R₁ is —H.

In some embodiments, in the conjugate of formula 1 and/or the conjugate of formula 2, R₁ is —H.

In some embodiments, in the conjugate of formula 1 and/or the conjugate of formula 2, R₃ is —H.

In some embodiments, in the conjugate of formula 1 and/or the conjugate of formula 2, R₂ is an optionally substituted phenyl.

In some embodiments, in the conjugate of formula 1 and/or the conjugate of formula 2, R₂ is

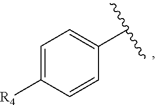

R₄ is selected from a group consisting of —OH, —PO₃H₂, —SeH, —SH, optionally substituted alkyl-OH, optionally substituted alkyl-halogen, optionally substituted alkyl-N₃, —B(OH)₂, -halogen, —OTf, optionally substituted alkyl-NH₂, —O-optionally substituted alkyl-C≡CH, —CO—NH—C≡CH-optionally substituted alkyl.

In some embodiments, in the conjugate of formula 1 and/or the conjugate of formula 2, R₂ is

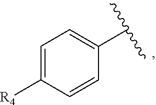

wherein R₄ is selected from a group consisting of: —OH, —PO₃H₂, —SeH, —SH, —CH₂OH, —CH₂Br, —CH₂N₃, —B(OH)₂, —Br, —OTf, —CH₂NH₂, —Cl, —OCH₂C≡CH or —CO—NH—C≡CH.

In some embodiments, in the conjugate of formula 1 and/or the conjugate of formula 2,R₂ is

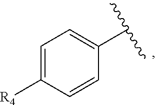

R₄, wherein R₄ is —O—(CH₂)ₙ₁—COO—R₅, n₁ is an integer of 1 to 10, wherein R₅ is selected from a group consisting of:

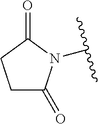 [SMILES: *N1C(=O)CCC1=O]

### and H.

In some embodiments, in the conjugate of formula 1 and/or the conjugate of formula 2,R₂ is

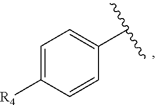

wherein R₄ is —O—(CH₂)ₙ₂—CO—NH—R₆, n₂ is an integer of 1 to 10, wherein R₆ is —(CH₂)ₙ₃—CO—R₇, n₃ is an integer of 1 to 10, wherein R₇ is selected from a group consisting of:

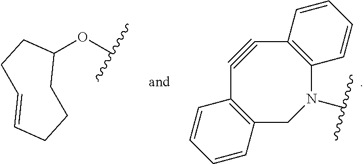 [SMILES: *OC1CC/C=C/CCC1 ; *N1Cc2ccccc2C#Cc2ccccc21]

In some embodiments, in the conjugate of formula 1 and/or the conjugate of formula 2, R₂ is

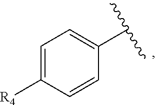

wherein R₄ is —O—(CH₂)ₙ₂—CO—NH—R₆, n₂ is an integer of 1 to 10, wherein R₆ is —(CH₂)ₙ₄-R₈, n₄ is an integer of 1 to 10, wherein R₈ is selected from a group consisting of:

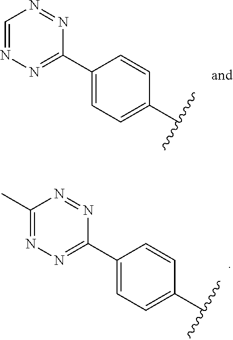 [SMILES: *c1ccc(-c2nncnn2)cc1 ; *c1ccc(-c2nnc(C)nn2)cc1]

In some embodiments, in the conjugate of formula 1 and/or the conjugate of formula 2, R₂ is

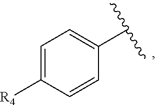

wherein R₄ is —O—(CH₂)ₙ₂—CO—NH—R₆, n₂ is an integer of 1 to 10, wherein R₆ is —(CH₂CH₂—O)ₙ₅—(CH₂)ₙ₆—NH—CO—O—R₉, n₈ is an integer of 1 to 10, n₆ is an integer of 1 to 10, wherein R₉ is selected from a group consisting of: H and

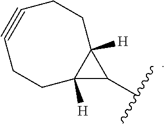 [SMILES: *C1[C@H]2CCC#CCC[C@@H]12]

In some embodiments, in the conjugate of formula 1 and/or the conjugate of formula 2, R₂ is

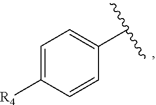

wherein R₄ is —(OCH₂CH₂)ₙ₇—O—(CH₂)ₙ₈—R₁₀, n₇ is an integer of 1 to 10, n₈ is an integer of 1 to 10, wherein R₁₀ is selected from a group consisting of: —COOH, —NH₂ and

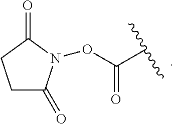 [SMILES: *C(=O)ON1C(=O)CCC1=O]

In some embodiments, in the conjugate of formula 1 and/or the conjugate of formula 2,R₂ is

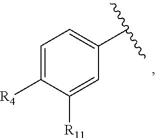

wherein R₄ is —(OCH₂CH₂)ₙ₇—O—(CH₂)ₙ₈—R₁₀, n₇ is an integer of 1 to 10, n₈ is an integer of 1 to 10, wherein R₁₀ is selected from a group consisting of: —COOH, —NH₂ and

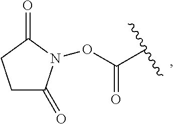

wherein R₁ is selected from a group consisting of: optionally substituted alkyl-halogen, optionally substituted alkyl-N and O—optionally substituted alkyl.

In some embodiments, in the conjugate of formula 1 and/or the conjugate of formula 2, R₂ is

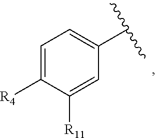

wherein R₄ is —(OCH₂CH₂)ₙ₇—O—(CH₂)ₙ₈—R₁₀, n₇ is an integer of 1 to 10, n₈ is an integer of 1 to 10, wherein R₁₀ is selected from a group consisting of: —COOH, —NH₂ and

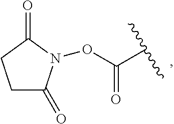

wherein R₁ is selected from a group consisting of: —CF₃, —CN and —OCH₃.

In some embodiments, in the conjugate of formula 1 and/or the conjugate of formula 2, R₂ is optionally substituted alkyl-CH═CH—R₁₂, wherein R₁₂ is

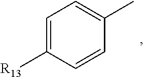

wherein R₁₃ is —CH₂N₃.

In some embodiments, in the conjugate of formula 1 and/or the conjugate of formula 2, said ring is an optionally substituted cycloolefin, or an optionally substituted aryl-cycloolefin.

In some embodiments, in the conjugate of formula 1 and/or the conjugate of formula 2,said ring is selected from the group consisting of:

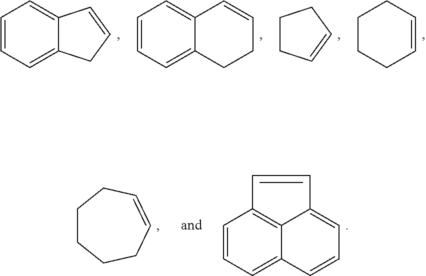

In some embodiments, in the conjugate of formula 1 and/or the conjugate of formula 2, L1 is selected from the group consisting of:

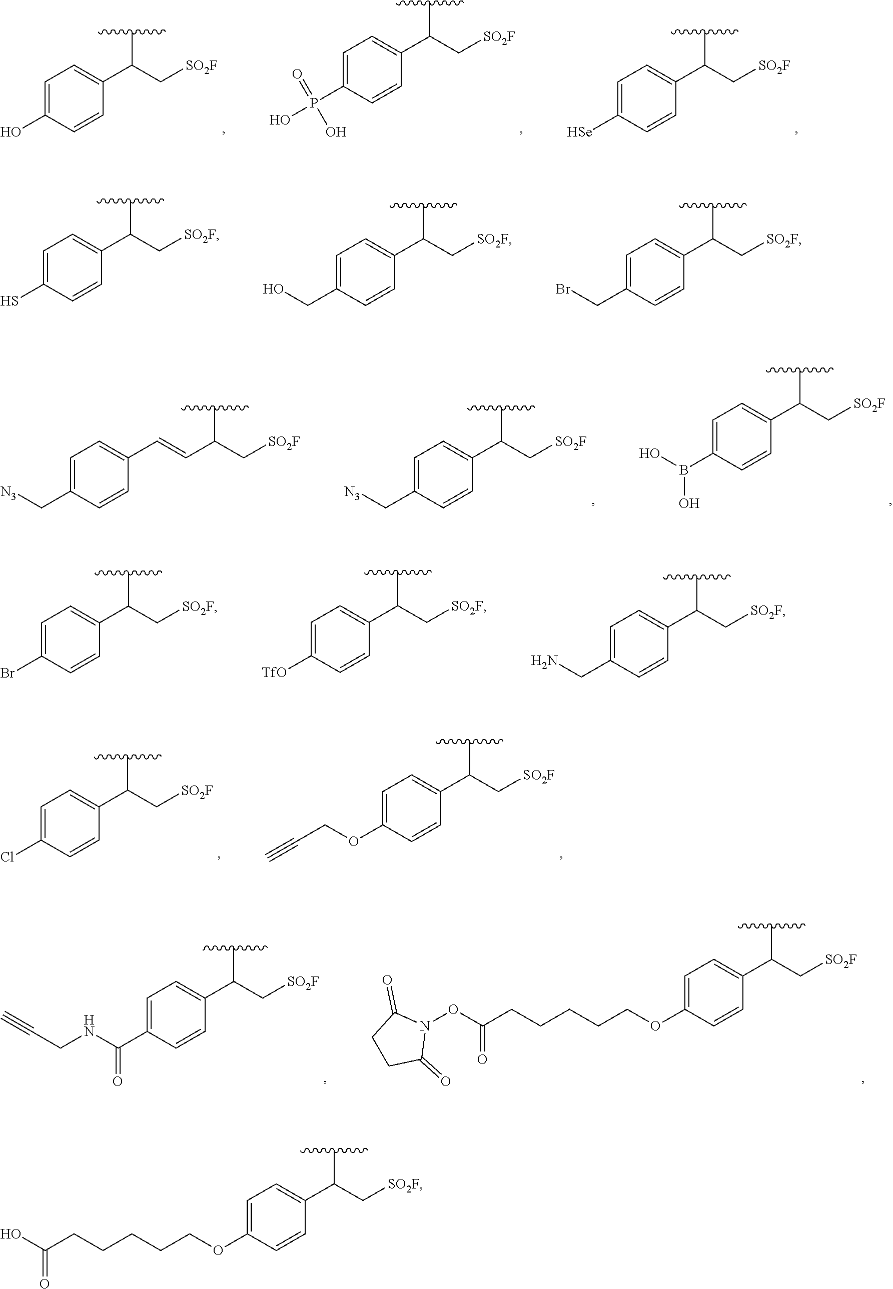

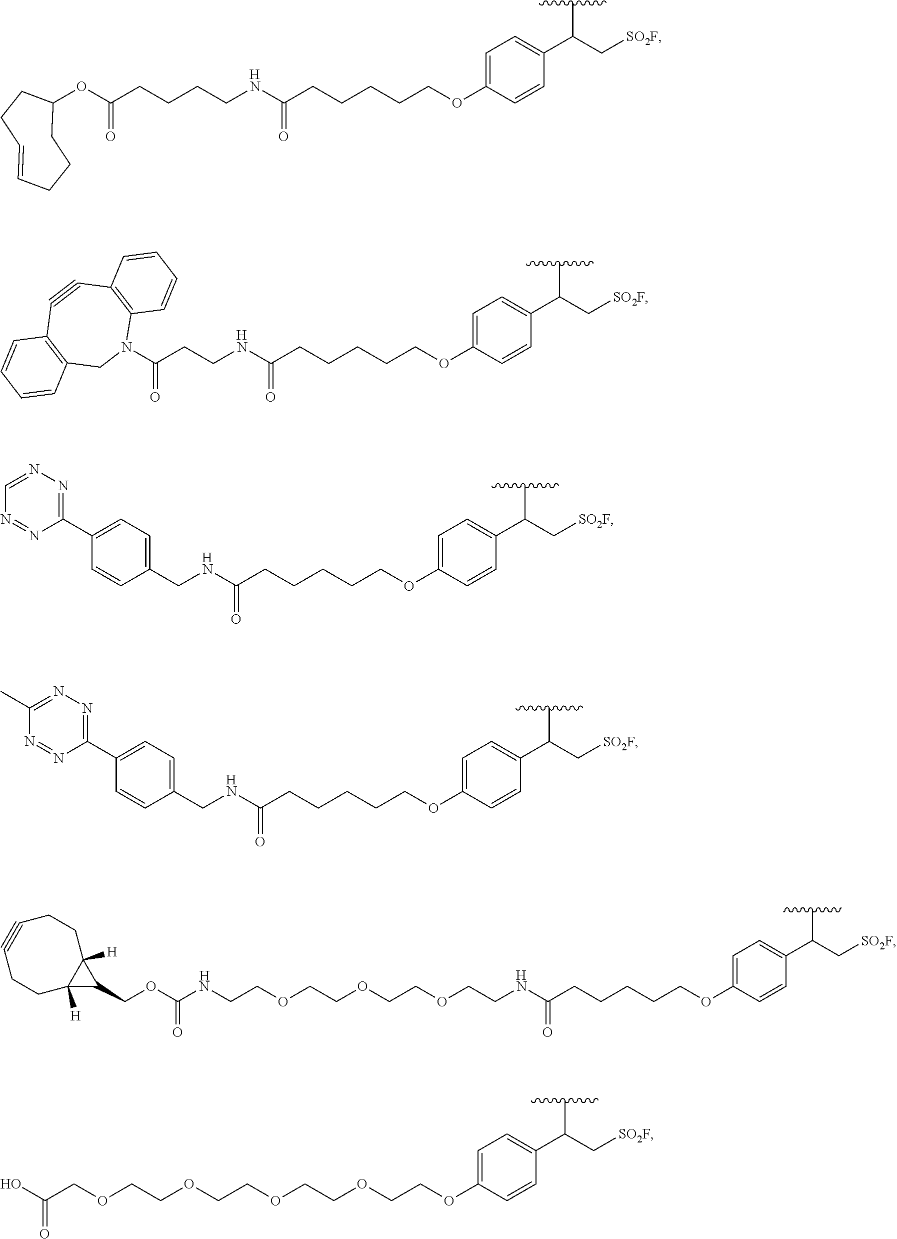

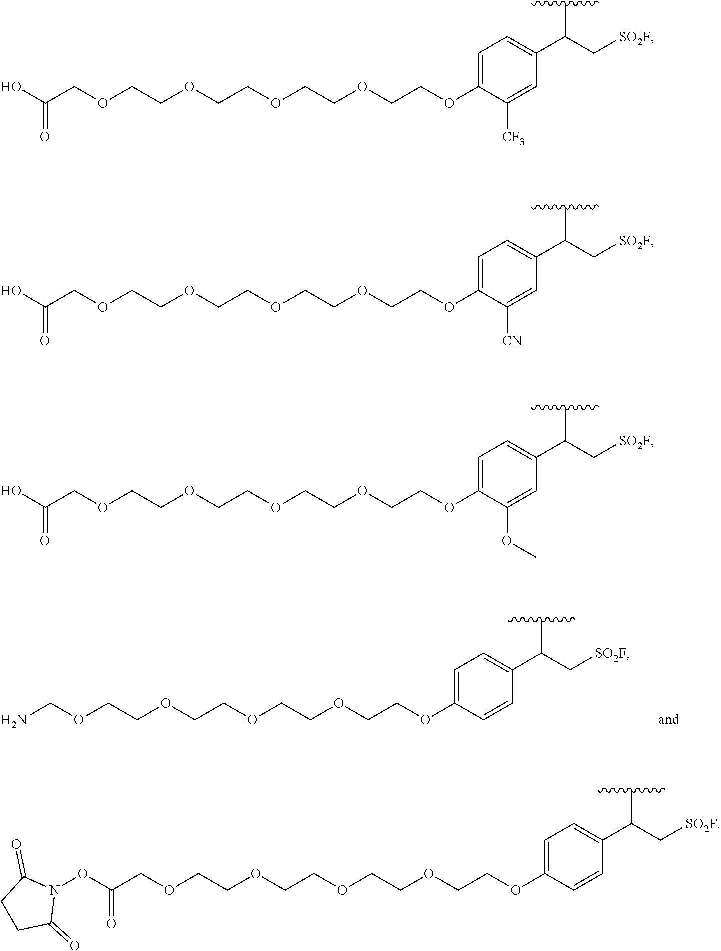 [SMILES: *C(CS(=O)(=O)F)c1ccc(OCCOCCOCCOCCOCC(=O)O)c(C(F)(F)F)c1 ; *C(CS(=O)(=O)F)c1ccc(OCCOCCOCCOCCOCC(=O)O)c(C#N)c1 ; *C(CS(=O)(=O)F)c1ccc(OCCOCCOCCOCCOCC(=O)O)c(OC)c1 ; *C(CS(=O)(=O)F)c1ccc(OCCOCCOCCOCCOCN)cc1 ; *C(CS(=O)(=O)F)c1ccc(OCCOCCOCCOCCOCC(=O)ON2C(=O)CCC2=O)cc1]

In some embodiments, in the conjugate of formula 1 and/or the conjugate of formula 2, said conjugate is selected from the group consisting of:

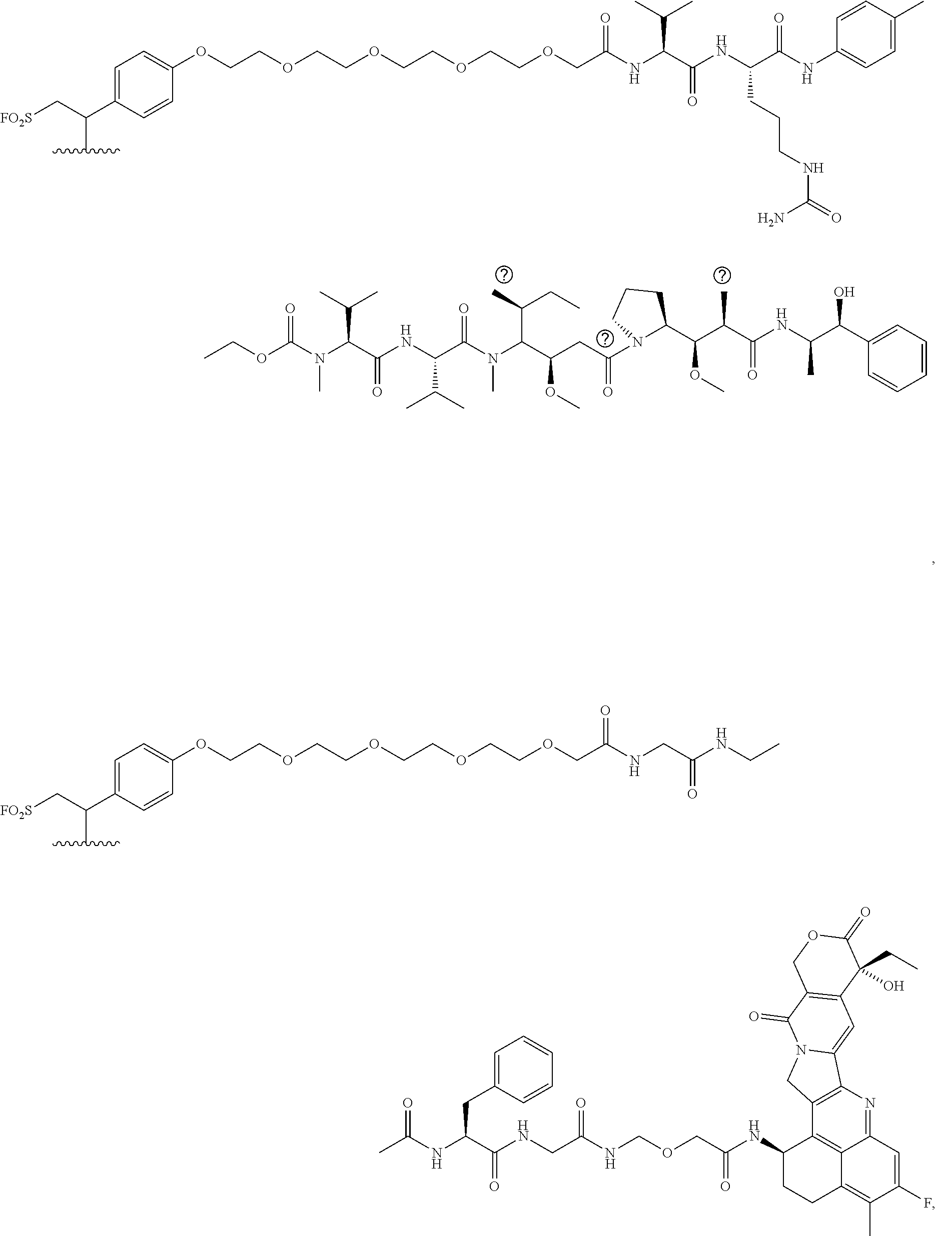

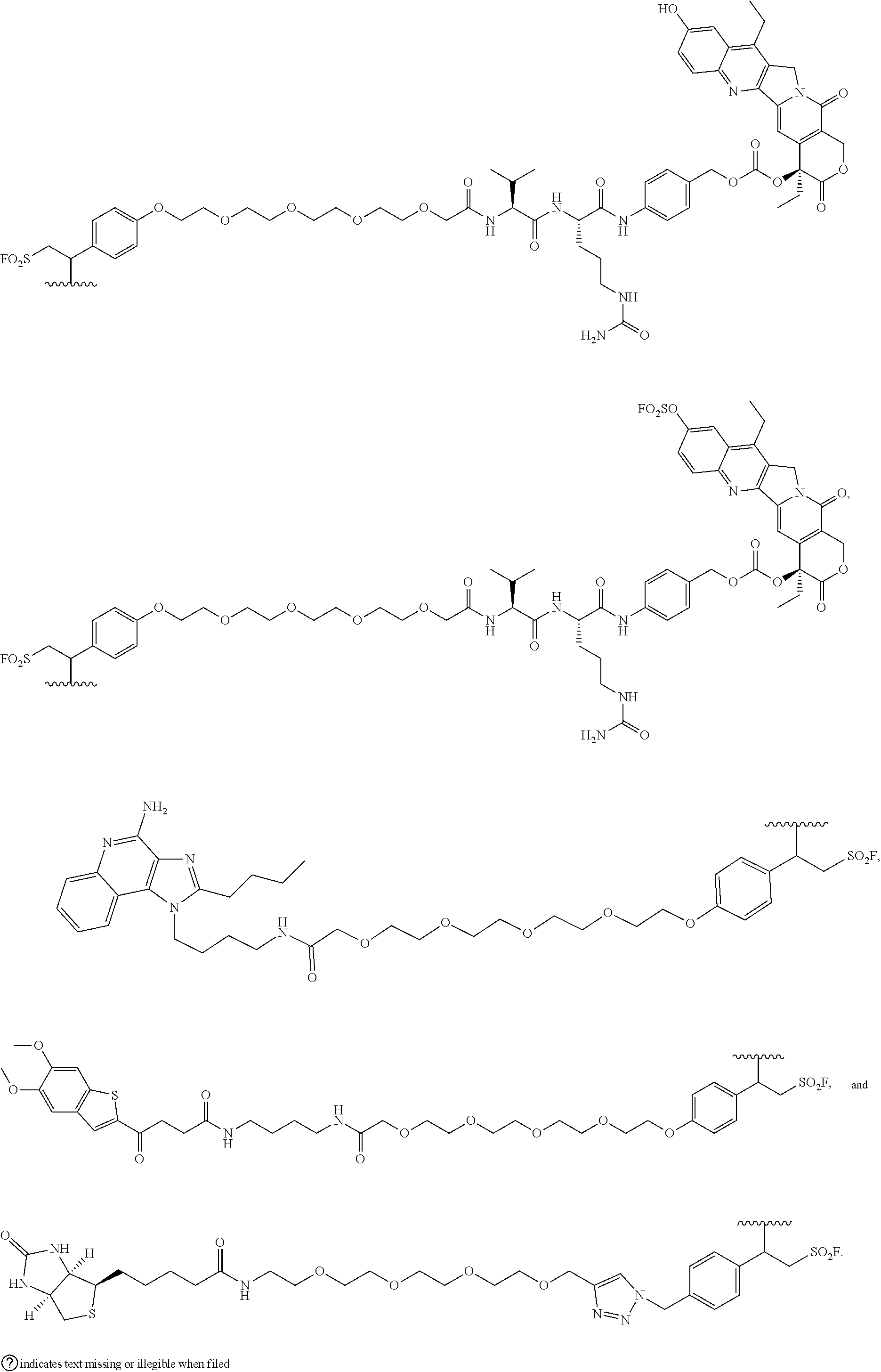

In another aspect, the present application provides a conjugate of formula 3, (L1)ₐ-(L2)_{b}-(D)_{c} 3, wherein L1 is a compound of formula III,

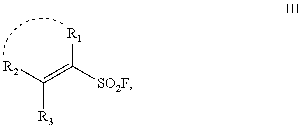

L2 is a linker, and L2 is linked to R₁, R₃ or R₂, D is a functional molecule, a is an integer of 1 to 10, b, c is each independently an integer of 0 to 10, provided that b and c are not simultaneously 0, wherein R₁ is H, an optionally substituted alkyl or an optionally substituted aryl, wherein R₂ is H, an optionally substituted alkyl or an optionally substituted aryl, wherein R₃ is H, an optionally substituted alkyl or an optionally substituted aryl, optionally, a C linking R₁ and a C linking R₂ form a ring.

In some embodiments, in the conjugate of formula 3, L2 is selected from the group consisting of a cleavable linker, a non-cleavable linker, a hydrophilic linker, a hydrophobic linker, a procharged linker, an uncharged linker and a dicarboxylic acid-based linker.

In some embodiments, in the conjugate of formula 3, L2 is selected from the group consisting of VC-PAB, N-succinimidyl-3-(2-pyridyldithio)propionate (SPDP), N-succinimidyl 4-(2-pyridyldithio)pentanoate (SPP), N-succinimidyl 4-(2-pyridyldithio)butanoate (SPDB), N-succinimidyl-4-(2-pyridyldithio)-2-sulfo-butanoate (sulfo-SPDB), N-succinimidyl iodoacetate (SIA), N-succinimidyl(4-iodoacetyl)aminobenzoate (SIAB), maleimide PEG NHS, N-succinimidyl 4-(maleimidomethyl) cyclohexanecarboxylate (SMCC), N-sulfosuccinimidyl 4-(maleimidomethyl) cyclohexanecarboxylate (sulfo-SMCC) or 2,5-dioxopyrrolidin-1-yl 17-(2,5-dioxo-2,5-dihydro-1H-pyrrol-1-yl)-5,8,11,14-tetraoxo-4,7,10,13-tetraazaheptadecan-1-oate (CX1-1).

In some embodiments, in the conjugate of formula 3, D has a biological function.

In some embodiments, in the conjugate of formula 3, D and/or a derivative thereof is capable of inhibiting the growth of a tumor cell.

In some embodiments, in the conjugate of formula 3, D is a drug.

In some embodiments, in the conjugate of formula 3, D is selected from the group consisting of a V-ATPase inhibitor, a pro-apoptotic agent, a Bcl2 inhibitor, an MCL1 inhibitor, a HSP90 inhibitor, an IAP inhibitor, an mTor inhibitor, a microtubule stabilizer, a microtubule destabilizer, an auristatin, a dolastatin, a maytansinoid, a MetAP (methionine aminopeptidase), an inhibitor of nuclear export of proteins CRM1, a DPPIV inhibitor, proteasome inhibitors, inhibitors of phosphoryl transfer reactions in mitochondria, a protein synthesis inhibitor, a kinase inhibitor, a CDK2 inhibitor, a CDK9 inhibitor, a kinesin inhibitor, an HDAC inhibitor, a DNA damaging agent, a DNA alkylating agent, a DNA intercalator, a DNA minor groove binder, a DHFR inhibitor, a nucleoside analog, a HD AC inhibitor; an anthracycline; a NAMPT inhibitor; a hydrophilic prodrug; a SN-38 glucuronide, a etoposide phosphate; a nitrogen mustard, a proteosome inhibitor, a cytokine, a Toll-like receptor agonist and a Sting agonist.

In some embodiments, in the conjugate of formula 3, D is MMAE or a derivative thereof; melphalan or a derivative thereof; lenalidomide or a derivative thereof; IL-2 or a derivative thereof; neoleukin-2/15 or a derivative thereof; T785 or a derivative thereof; or, MSA-2 or a derivative thereof.

In some embodiments, in the conjugate of formula 3, R₁ is —H.

In some embodiments, in the conjugate of formula 3, R₃ is —H.

In some embodiments, in the conjugate of formula 3, R₂ is an optionally substituted phenyl.

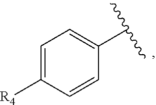

In some embodiments, in the conjugate of formula 3, R₂ is R_{4 l}, wherein R₄ is selected from a group consisting of —OH, —PO3H₂, —SeH, —SH, optionally substituted alkyl-OH, optionally substituted alkyl-halogen, optionally substituted alkyl-N₃, —B(OH)₂, -halogen, —OTf, optionally substituted alkyl-NH₂, —O-optionally substituted alkyl-C≡CH, —CO—NH—C≡CH-optionally substituted alkyl.

In some embodiments, in the conjugate of formula 3, R₂ is

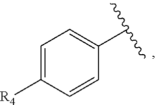

wherein R₄ is selected from a group consisting of —OH, —PO₃H₂, —SeH, —SH, —CH₂OH, —CH₂Br, —CH₂N₃, —B(OH)₂, —Br, —OTf, —CH₂NH₂, —Cl, —OCH₂C≡CH or —CO—NH—C≡CH.

In some embodiments, in the conjugate of formula 3, R₂ is

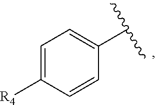

wherein R₄ is —O—(CH₂)ₙ₁—COO—R₅, n₁ is an integer of 1 to 10, wherein R₈ is selected from a group consisting of:

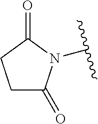 [SMILES: *N1C(=O)CCC1=O]

### and —H.

In some embodiments, in the conjugate of formula 3, R₂ is

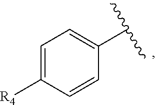

wherein R₄ is —O—(CH₂)ₙ₂—CO—NH—R₆, n₂ is an integer of 1 to 10, wherein R₆ is —(CH₂)ₙ₃—CO—R₇, n₃ is an integer of 1 to 10, wherein R₇ is selected from a group consisting of

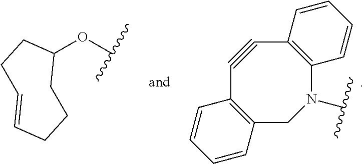 [SMILES: *OC1CC/C=C/CCC1 ; *N1Cc2ccccc2C#Cc2ccccc21]

In some embodiments, in the conjugate of formula 3, R₂ is

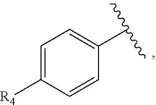

wherein R₄ is —O—(CH₂)ₙ₂—CO—NH—R₆, n₂ is an integer of 1 to 10, wherein R₆ is —(CH₂)ₙ₄-R₈, n₄ is an integer of 1 to 10, wherein R₈ is selected from a group consisting of:

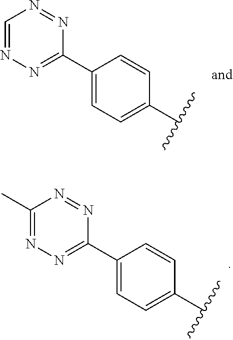 [SMILES: *c1ccc(-c2nncnn2)cc1 ; *c1ccc(-c2nnc(C)nn2)cc1]

In some embodiments, in the conjugate of formula 3, R₂ is

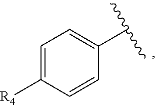

wherein R₄ is —O—(CH₂)ₙ₂—CO—NH—R₆, n₂ is an integer of 1 to 10, wherein R₆ is —(CH₂CH₂—O)ₙ₅—(CH₂)ₙ₆—NH—CO—O—R₉, n₈ is an integer of 1 to 10, n₆ is an integer of 1 to 10, wherein R₉ is selected from a group consisting of: H and

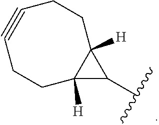 [SMILES: *C1[C@H]2CCC#CCC[C@@H]12]

In some embodiments, in the conjugate of formula 3, R₂ is

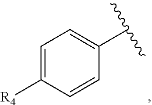

wherein R₄ is —(OCH₂CH₂)ₙ₇—O—(CH₂)ₙ₈—R₁₀, n₇ is an integer of 1 to 10, n₈ is an integer of 1 to 10, wherein R₁₀ is selected from a group consisting of: —COOH, —NH₂ and

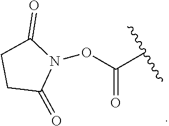 [SMILES: *C(=O)ON1C(=O)CCC1=O]

In some embodiments, in the conjugate of formula 3, R₂ is

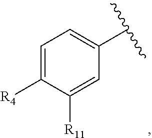

wherein R₄ is —(OCH₂CH₂)ₙ₇—O—(CH₂)ₙ₈—R₁₀, n₇ is an integer of 1 to 10, n₈ is an integer of 1 to 10, wherein R₁₀ is selected from a group consisting of: —COOH, —NH₂ and

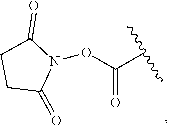

wherein R₁₁ is selected from a group consisting of: optionally substituted alkyl-halogen, optionally substituted alkyl-N and O—optionally substituted alkyl.

In some embodiments, in the conjugate of formula 3, R₂ is

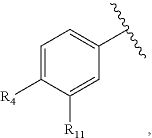

wherein R₄ is —(OCH₂CH₂)ₙ₇—O—(CH₂)ₙ₈—R₁₀, n₇ is an integer of 1 to 10, n₈ is an integer of 1 to 10, wherein R₁₀ is selected from a group consisting of: —COOH, —NH₂ and

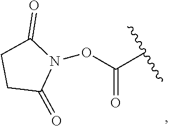

wherein R₁₁ is selected from a group consisting of: —CF₃, —CN and —OCH₃.

In some embodiments, in the conjugate of formula 3, R₂ is optionally substituted alkyl-CH═CH—R₁₂, wherein R₁₂ is

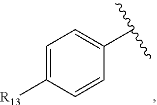

wherein R₁₃ is —CH₂N₃.

In some embodiments, in the conjugate of formula 3, said ring is an optionally substituted cycloolefin, or an optionally substituted aryl-cycloolefin.

In some embodiments, in the conjugate of formula 3, said ring is selected from the group consisting of:

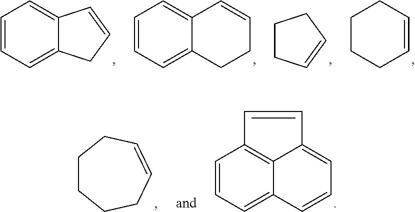

In some embodiments, in the conjugate of formula 3, said L1 is selected from the group consisting of:

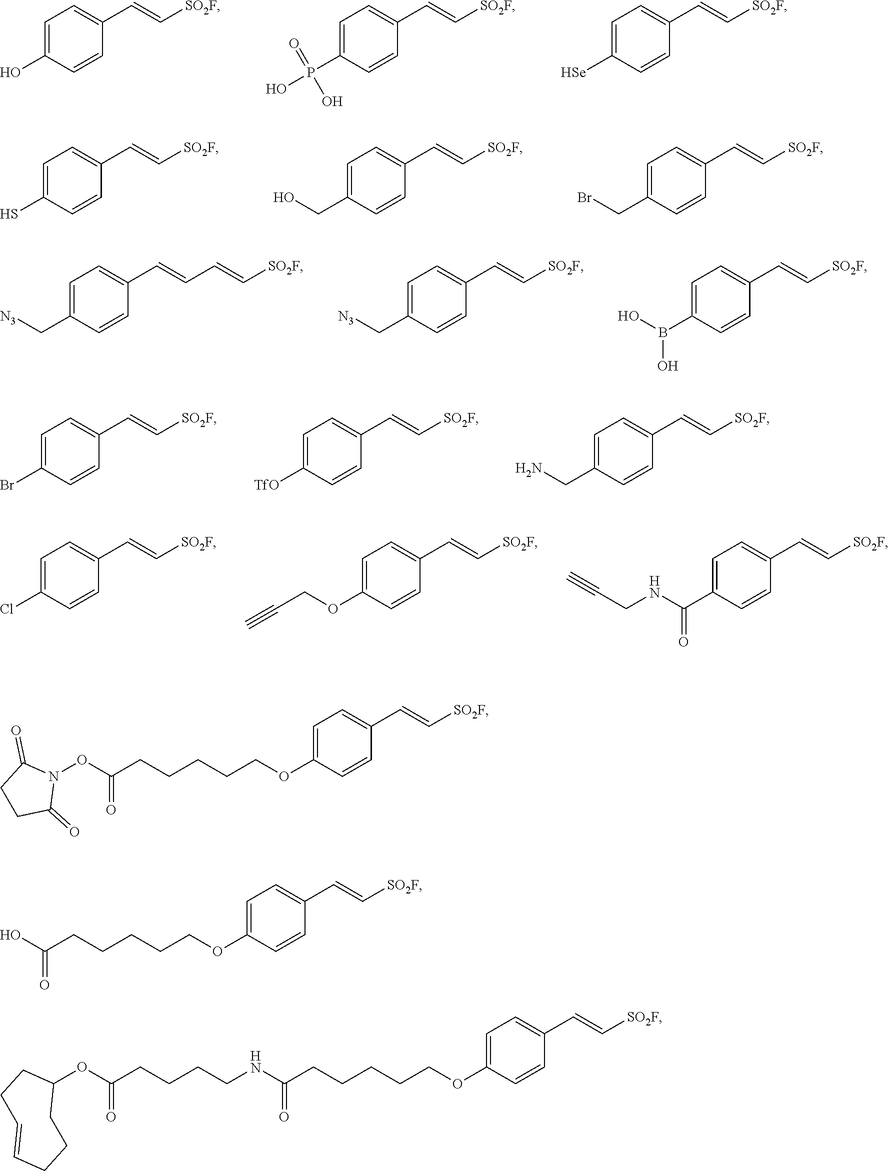

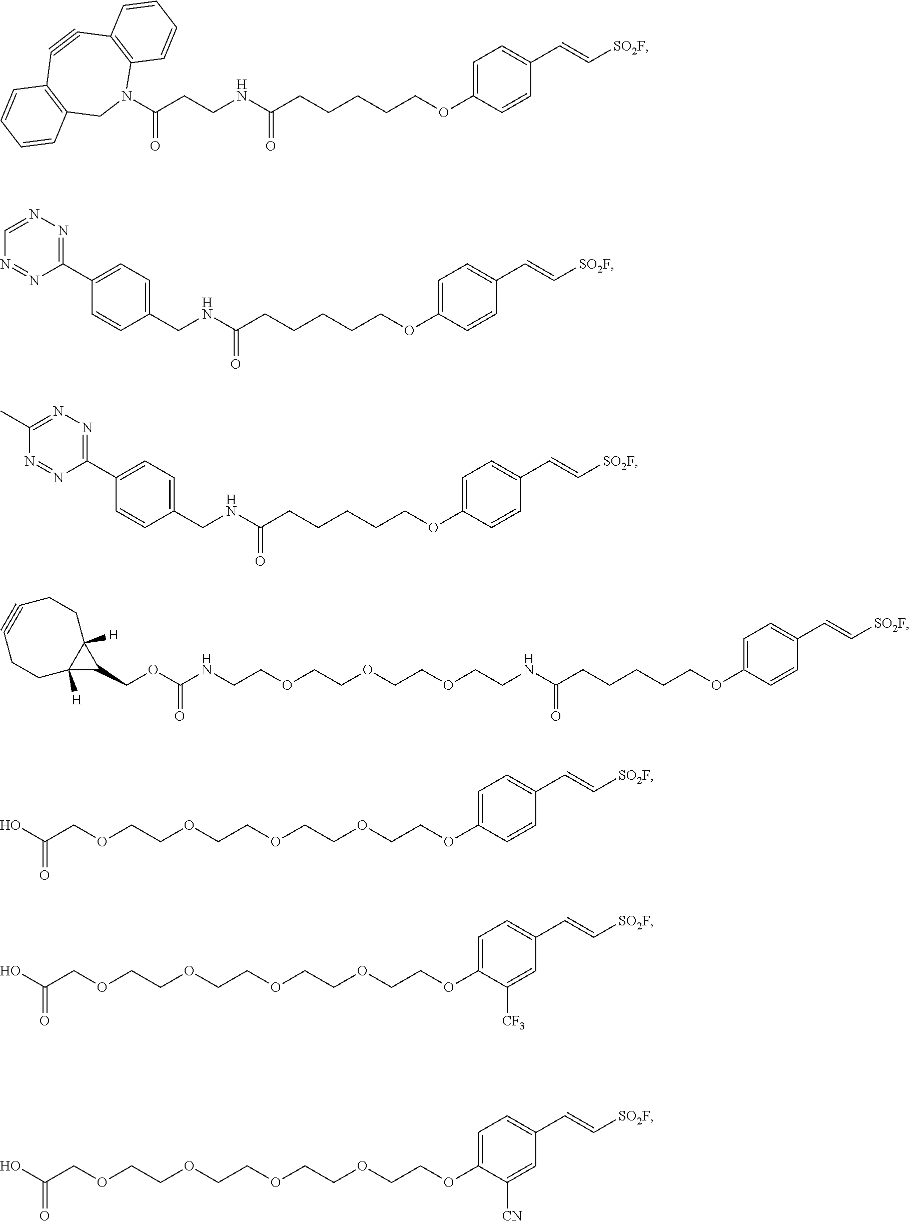

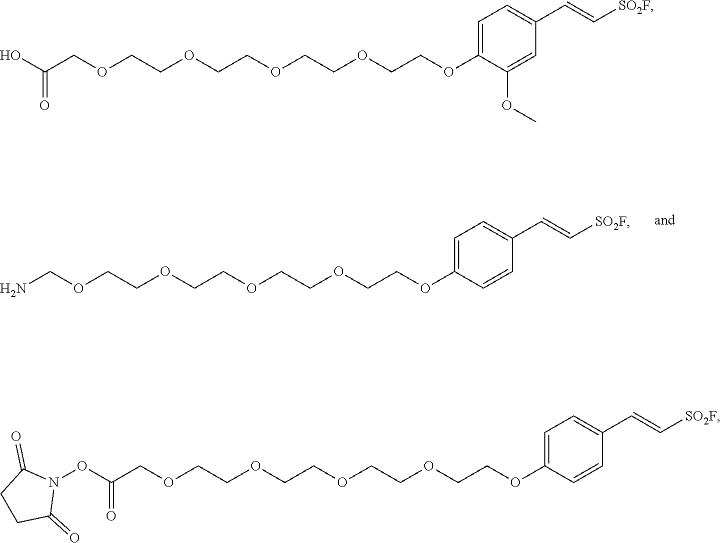 [SMILES: COc1cc(/C=C/S(=O)(=O)F)ccc1OCCOCCOCCOCCOCC(=O)O ; NCOCCOCCOCCOCCOc1ccc(/C=C/S(=O)(=O)F)cc1 ; O=C(COCCOCCOCCOCCOc1ccc(/C=C/S(=O)(=O)F)cc1)ON1C(=O)CCC1=O]

In some embodiments, in the conjugate of formula 3, said conjugate is selected from the group consisting of:

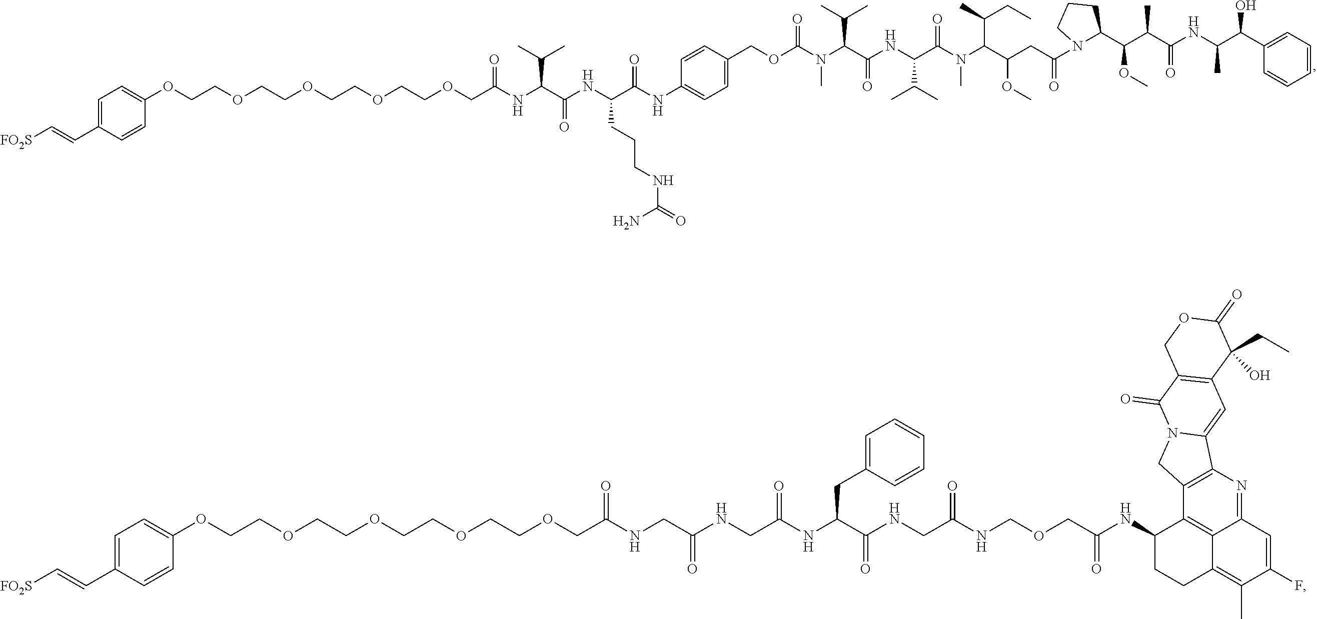

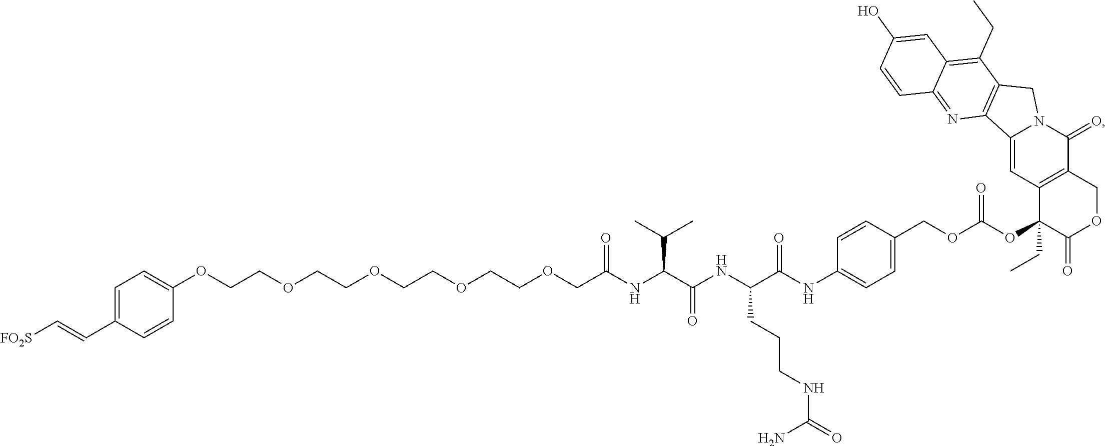

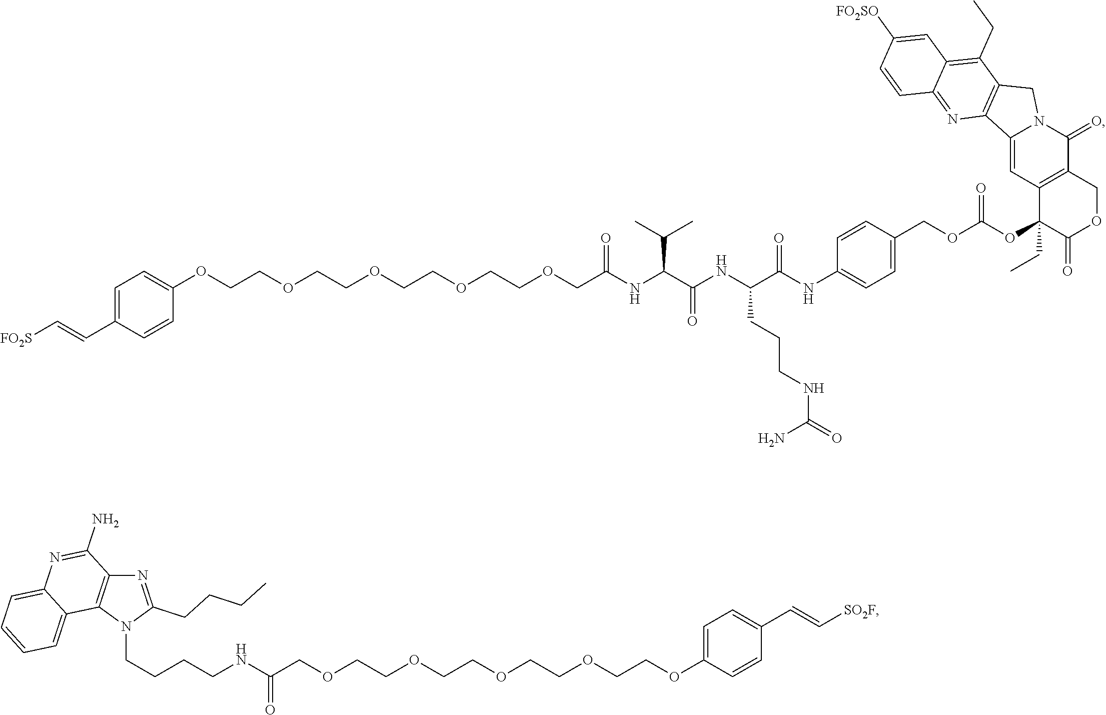

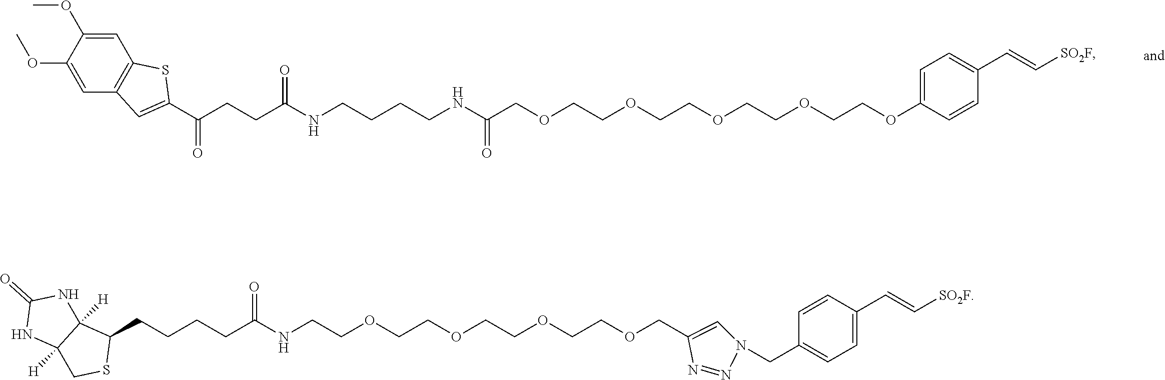 [SMILES: COc1cc2cc(C(=O)CCC(=O)NCCCCNC(=O)COCCOCCOCCOCCOc3ccc(/C=C/S(=O)(=O)F)cc3)sc2cc1OC ; O=C(CCCC[C@H]1SC[C@H]2NC(=O)N[C@H]21)NCCOCCOCCOCCOCc1cn(Cc2ccc(/C=C/S(=O)(=O)F)cc2)nn1]

In another aspect, the present application provides a method for preparing a conjugate, comprising the following steps: obtaining a conjugate of formula 1:

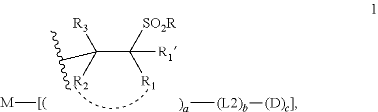

by conjugating a conjugate of formula 3:

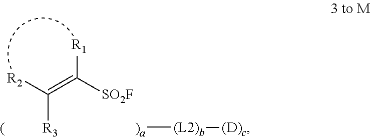

wherein M is a biological macromolecule, and M is linked to L1 with a nucleophilic functional group of M, L2 is a linker, and L2 is linked to R₁, R₃ or R₂ in the formula 1, D is a functional molecule, a is an integer of 1 to 10, b, c is each independently an integer of 0 to 10, provided that b and c are not simultaneously 0, wherein R₁ is H, an optionally substituted alkyl or an optionally substituted aryl, wherein R₁′ is H or the isotope thereof, wherein R₂ is H, an optionally substituted alkyl or an optionally substituted aryl, wherein R₃ is H, an optionally substituted alkyl or an optionally substituted aryl, optionally, a C linking R₁ and a C linking R₂ form a ring.

In some embodiments, in the method, said nucleophilic functional group of M is selected from a group consisting of —SH, —NH₂, —SeH, —OH, and

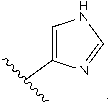 [SMILES: *c1c[nH]cn1]

A method for preparing a conjugate, comprising the following steps: obtaining a conjugate of formula 2:

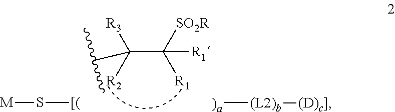

formula 3:

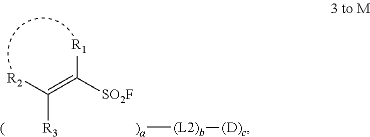

wherein M-S is a biological macromolecule with a cysteine, M-S is linked to L1 with the cysteine, L2 is a linker, and L2 is linked to R₁, R₃ or R₂ in the formula 3, D is a functional molecule, a is an integer of 1 to 10, b, c is each independently an integer of 0 to 10, provided that b and c are not simultaneously 0, wherein R₁ is H, an optionally substituted alkyl or an optionally substituted aryl, wherein R₁′ is H or the isotope thereof, wherein R₂ is H, an optionally substituted alkyl or an optionally substituted aryl, wherein R₃ is H, an optionally substituted alkyl or an optionally substituted aryl, optionally, a C linking R₁ and a C linking R₂ form a ring.

In some embodiments, in the method, M is selected from a group consisting of a protein, a DNA, a RNA, and a virus.

In some embodiments, in the method, M is a biological macromolecule expressed on the surface of a cell.

In some embodiments, in the method, M is an antigen binding protein or a fragment thereof.

In some embodiments, in the method, M is a monoclonal antibody, a chimeric antibody, a humanized antibody, a human engineered antibody, a human antibody, a single chain antibody(scFv) or an antibody fragment.

In some embodiments, in the method, M comprises a functional group for a nucleophilic addition reaction.

In some embodiments, in the method, L2 is selected from the group consisting of a cleavable linker, a non-cleavable linker, a hydrophilic linker, a hydrophobic linker, a procharged linker, an uncharged linker and a dicarboxylic acid-based linker.

In some embodiments, in the method, L2 is selected from the group consisting of VC-PAB, N-succinimidyl-3-(2-pyridyldithio)propionate (SPDP), N-succinimidyl 4-(2-pyridyldithio)pentanoate (SPP), N-succinimidyl 4-(2-pyridyldithio)butanoate (SPDB), N-succinimidyl-4-(2-pyridyldithio)-2-sulfo-butanoate (sulfo-SPDB), N-succinimidyl iodoacetate (SIA), N-succinimidyl(4-iodoacetyl)aminobenzoate (SIAB), maleimide PEG NHS, N-succinimidyl 4-(maleimidomethyl) cyclohexanecarboxylate (SMCC), N-sulfosuccinimidyl 4-(maleimidomethyl) cyclohexanecarboxylate (sulfo-SMCC) or 2,5-dioxopyrrolidin-1-yl 17-(2,5-dioxo-2,5-dihydro-1H-pyrrol-1-yl)-5,8,11,14-tetraoxo-4,7,10,13-tetraazaheptadecan-1-oate (CX1-1).

In some embodiments, in the method, D has a biological function.

In some embodiments, in the method, D and/or a derivative thereof is capable of inhibiting the growth of a tumor cell.

In some embodiments, in the method, D is a drug.

In some embodiments, in the method, D is selected from the group consisting of a V-ATPase inhibitor, a pro-apoptotic agent, a Bcl2 inhibitor, an MCL1 inhibitor, a HSP90 inhibitor, an IAP inhibitor, an mTor inhibitor, a microtubule stabilizer, a microtubule destabilizer, an auristatin, a dolastatin, a maytansinoid, a MetAP (methionine aminopeptidase), an inhibitor of nuclear export of proteins CRM1, a DPPIV inhibitor, proteasome inhibitors, inhibitors of phosphoryl transfer reactions in mitochondria, a protein synthesis inhibitor, a kinase inhibitor, a CDK2 inhibitor, a CDK9 inhibitor, a kinesin inhibitor, an HDAC inhibitor, a DNA damaging agent, a DNA alkylating agent, a DNA intercalator, a DNA minor groove binder and a DHFR inhibitor, a nucleoside analog, a HD AC inhibitor; an anthracycline; a NAMPT inhibitor; a hydrophilic prodrug; a SN-38 glucuronide, a etoposide phosphate; a nitrogen mustard, a proteosome inhibitor, a cytokine, a Toll-like receptor agonist and a Sting agonist.

In some embodiments, in the method, D is MMAE or a derivative thereof; melphalan or a derivative thereof; lenalidomide or a derivative thereof; IL-2 or a derivative thereof; neoleukin-2/15 or a derivative thereof; T785 or a derivative thereof; or, MSA-2 or a derivative thereof.

In some embodiments, in the method, R₁ is —H.

In some embodiments, in the method, R₃ is —H.

In some embodiments, in the method, R₂ is an optionally substituted phenyl.

In some embodiments, in the method, R₂ is

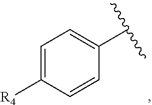

wherein R₄ is selected from a group consisting of —OH, —PO₃H₂, —SeH, —SH, optionally substituted alkyl-OH, optionally substituted alkyl-halogen, optionally substituted alkyl-N₃, —B(OH)₂, -halogen, —OTf, optionally substituted alkyl-NH₂, —O-optionally substituted alkyl-C≡CH, —CO—NH—C≡CH— optionally substituted alkyl.

In some embodiments, in the method, R₂ is

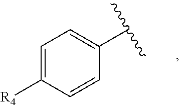

wherein R₄ is selected from a group consisting of —OH, —PO₃H₂, —SeH, —SH, —CH₂OH, —CH₂Br, —CH₂N₃, —B(OH)₂, —Br, —OTf, —CH₂NH₂, —Cl, —OCH₂C≡CH or —CO—NH—C≡CH.

In some embodiments, in the method, R₂ is

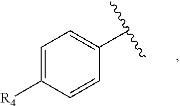

wherein R₄ is —O—(CH₂)ₙ₁—COO—R₅, n₁ is an integer of 1 to 10, wherein R₅ is selected from a group consisting of:

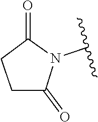 [SMILES: *N1C(=O)CCC1=O]

and —

In some embodiments, in the method, R₂ is

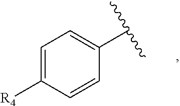

wherein R₄ is —O—(CH₂)ₙ₂—CO—NH—R₆, n₂ is an integer of 1 to 10, wherein R₆ is —(CH₂)ₙ₃—CO—R₇, n₃ is an integer of 1 to 10, wherein R₇ is selected from a group consisting of:

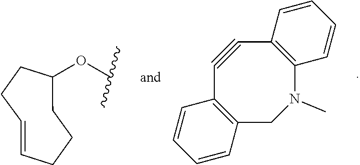 [SMILES: *OC1CC/C=C/CCC1 ; "and" ; CN1Cc2ccccc2C#Cc2ccccc21 ; "."]

In some embodiments, in the method, R₂ is

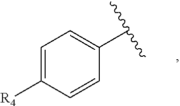

wherein R₄ is —O—(CH₂)ₙ₂—CO—NH—R₆, n₂ is an integer of 1 to 10, wherein R₆ is —(CH₂)ₙ₄-R₈, n₄ is an integer of 1 to 10, wherein R₈ is selected from a group consisting of:

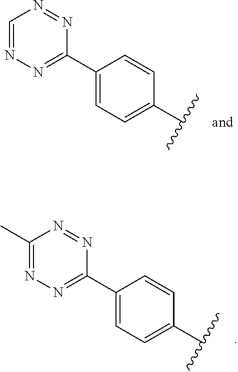 [SMILES: *c1ccc(-c2nncnn2)cc1 ; "and" ; *c1ccc(-c2nnc(C)nn2)cc1 ; "."]

In some embodiments, in the method, R₂ is

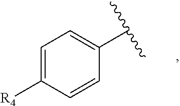

wherein R₄ is —O—(CH₂)ₙ₂—CO—NH—R₆, n₂ is an integer of 1 to 10, wherein R₆ is —(CH₂CH₂—O)ₙ₅—(CH₂)ₙ₆—NH—CO—O—R₉, n₈ is an integer of 1 to 10, n₆ is an integer of 1 to 10, wherein R₉ is selected from a group consisting of: H and

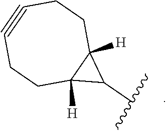 [SMILES: *C1[C@H]2CCC#CCC[C@@H]12 ; "."]

In some embodiments, in the method, R₂ is

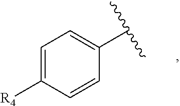

wherein R₄ is —(OCH₂CH₂)ₙ₇—O—(CH₂)ₙ₈—R₁₀, n₇ is an integer of 1 to 10, n₈ is an integer of 1 to 10, wherein R₁₀ is selected from a group consisting of: —COOH, —NH₂ and

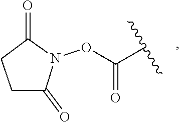 [SMILES: *C(=O)ON1C(=O)CCC1=O ; ","]

In some embodiments, in the method, R₂ is

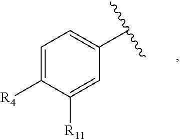

wherein R₄ is —(OCH₂CH₂)ₙ₇—O—(CH₂)ₙ₈—R₁₀, n₇ is an integer of 1 to 10, n₈ is an integer of 1 to 10, wherein R₁₀ is selected from a group consisting of: —COOH, —NH₂ and

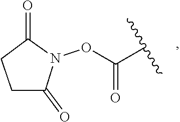

wherein R₁₁ is selected from a group consisting of: optionally substituted alkyl-halogen, optionally substituted alkyl-N and 0-optionally substituted alkyl.

In some embodiments, in the method, R₂ is

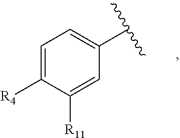

wherein R₄ is —(OCH₂CH₂)ₙ₇—O—(CH₂)ₙ₈—R₁₀, n₇ is an integer of 1 to 10, n₈ is an integer of 1 to 10, wherein R₁₀ is selected from a group consisting of: —COOH, —NH₂ and

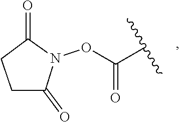

wherein R₁₁ is selected from a group consisting of: —CF₃, —CN and —OCH₃.

In some embodiments, in the method, R₂ is optionally substituted alkyl-CH═CH—R₁₂, wherein R₁₂ is

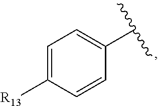

wherein R₁₃ is —CH₂N₃.

In some embodiments, in the method, said ring is an optionally substituted cycloolefin, or an optionally substituted aryl-cycloolefin.

In some embodiments, in the method, said ring is selected from the group consisting of:

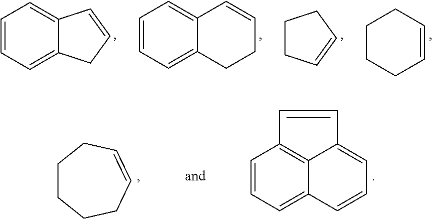

In some embodiments, the method is conducted at a temperature in a range of about 16° C. to about 37° C.

In some embodiments, the method is conducted at a pH in a range of about 7.4 to about 8.

In some embodiments, the method is conducted with a catalyst.

In some embodiments, the method further comprises the following step: purifying said conjugate of formula 3.

In another aspect, the present application provides a compound of formular III, or a pharmaceutically acceptable salt thereof:

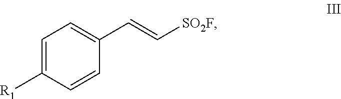

wherein R₁ is selected from a group consisting of: —OH, —PO₃H₂, —SeH, —SH, optionally substituted alkyl-OH, optionally substituted alkyl-halogen, optionally substituted alkyl-N₃, —B(OH)₂, -halogen, —OTf, optionally substituted alkyl-NH₂, —O-optionally substituted alkyl-C≡CH, —CO—NH—C≡CH-optionally substituted alkyl.

In some embodiments, R₁ is selected from a group consisting of: —OH, —PO₃H₂, —SeH, —SH, —CH₂OH, —CH₂Br, —CH₂N₃, —B(OH)₂, —Br, —OTf, —CH₂NH₂, —Cl, —OCH₂C≡CH or —CO—NH—C≡CH.

In some embodiments, the compound is selected from the group consisting of:

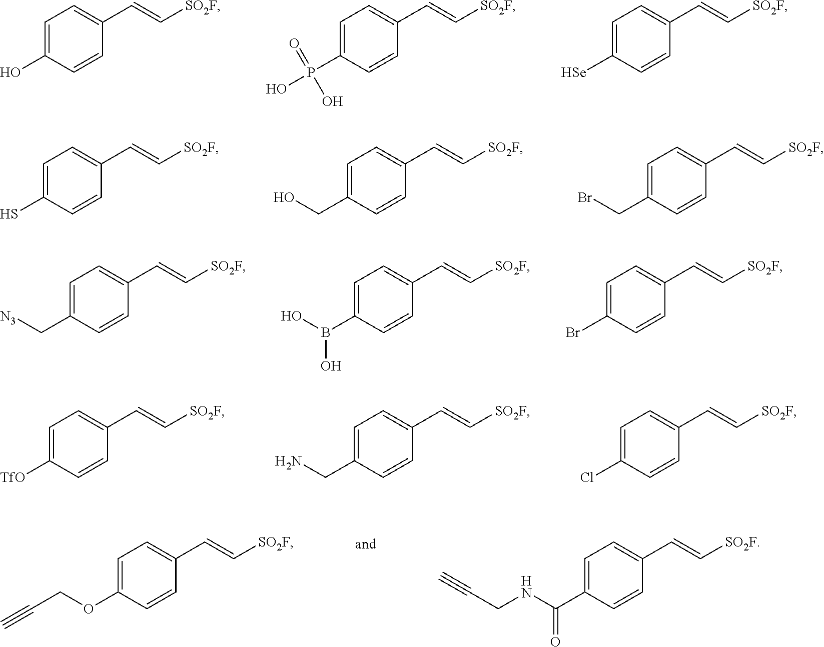

In another aspect, the present application provides a compound of formular IV, or a pharmaceutically acceptable salt thereof:

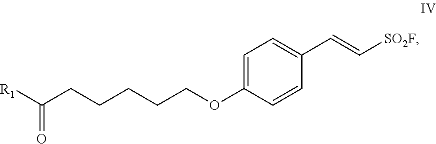

wherein R₁ is selected from a group consisting of NH—(CH₂)ₙ₁—CO—R₂, —OH, NH—(CH₂)ₙ₂-R₃, NH—(CH₂CH₂—O)ₙ₃—(CH₂)ₙ₄—NH—CO—O—R₄, or

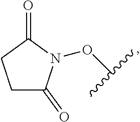

wherein n₁, n₂, n₃, or n₄ is independently an integer of 1 to 10, wherein R₂ is selected from a group consisting of;

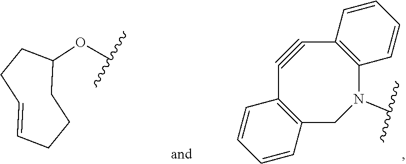

wherein R₃ is selected from a group consisting of:

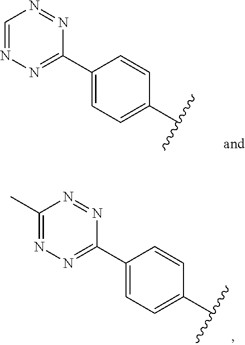 [SMILES: *c1ccc(-c2nncnn2)cc1 ; *c1ccc(-c2nnc(C)nn2)cc1]

wherein R₄ is

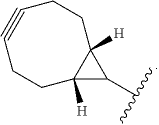 [SMILES: *C1[C@H]2CCC#CCC[C@@H]12]

In some embodiments, R₁ is selected from a group consisting of: NH—(CH₂)₂—CO—R₂, —OH, NH—(CH₂)—R₃, NH—(CH₂CH₂—O)₃—(CH₂)₂—NH—CO—O—R₄.

In some embodiments, the compound is selected from the group consisting of:

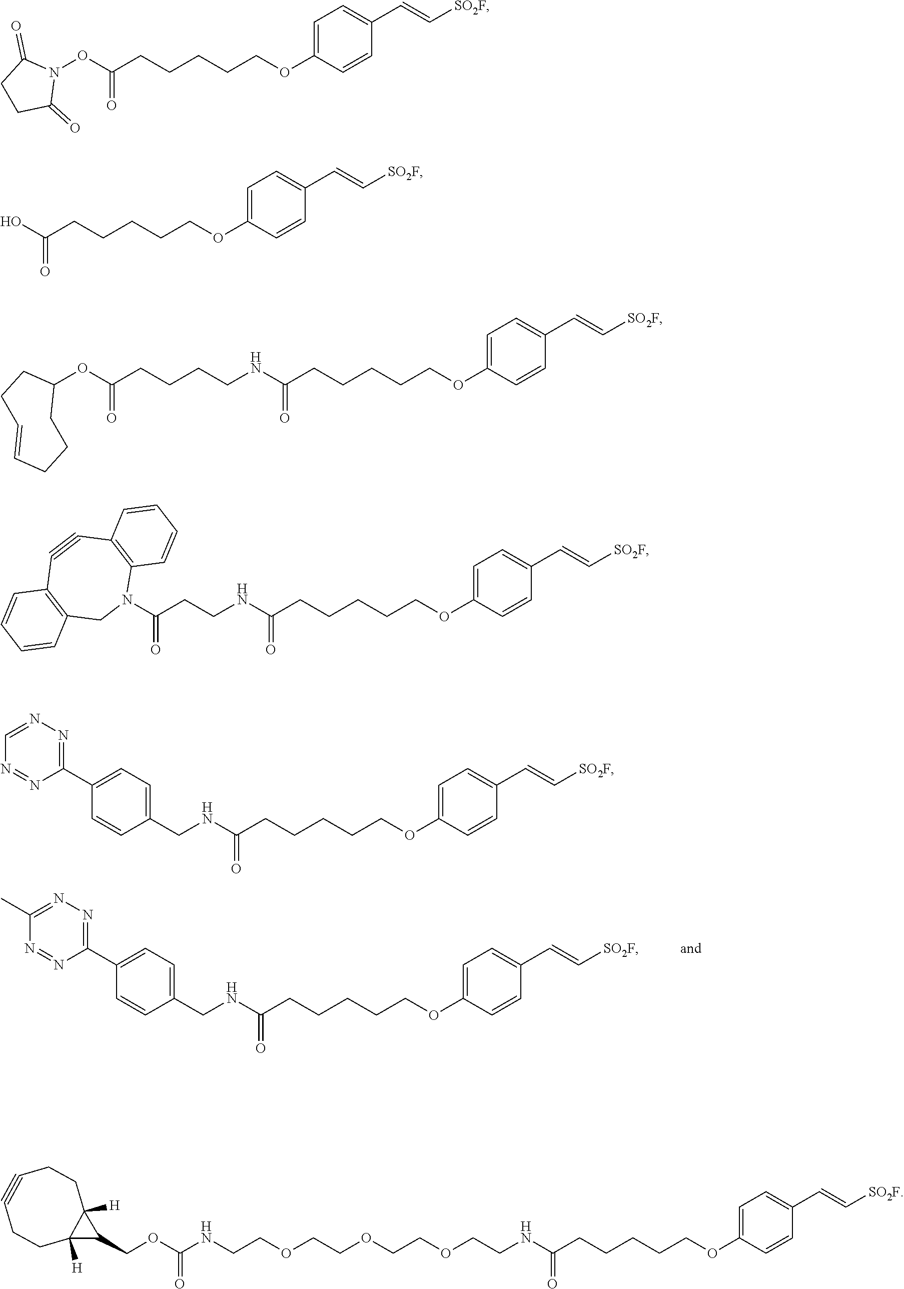

In another aspect, the present application provides a compound of Formular V, or a pharmaceutically acceptable salt thereof:

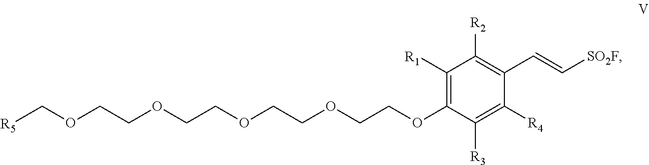

wherein R₁, R₂ and R₄ are any substituent, wherein R₃ is selected from a group consisting of: H, an optionally substituted alkyl-F₃, an optionally substituted alkyl-N or O—an optionally substituted alkyl, wherein R₅ is selected from a group consisting of: —COOH, —NH₂ and

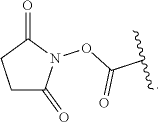 [SMILES: *C(=O)ON1C(=O)CCC1=O]

In some embodiments, R₁ is H.

In some embodiments, R₂ is H.

In some embodiments, R₄ is H.

In some embodiments, R₃ is selected from the group consisting of: H, CF₃, CN, and OCH₃.

In some embodiments, the compound is selected from the group consisting of:

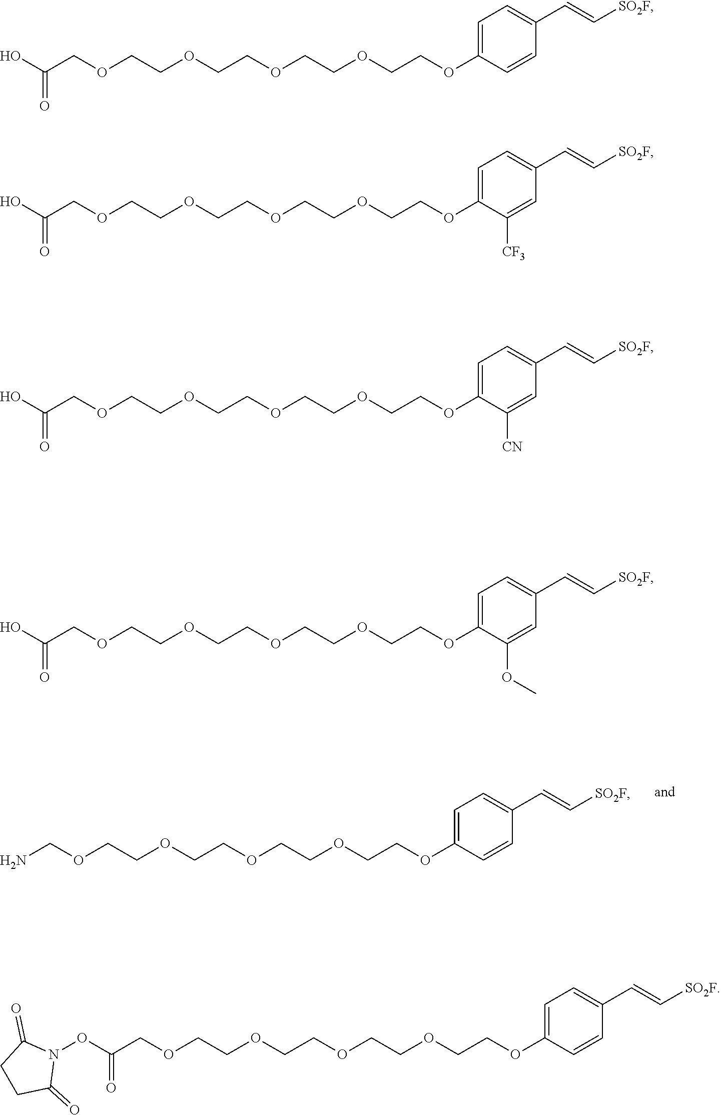 [SMILES: O=C(O)COCCOCCOCCOCCOc1ccc(/C=C/S(=O)(=O)F)cc1 ; O=C(O)COCCOCCOCCOCCOc1ccc(/C=C/S(=O)(=O)F)cc1C(F)(F)F ; N#Cc1cc(/C=C/S(=O)(=O)F)ccc1OCCOCCOCCOCCOCC(=O)O ; COc1cc(/C=C/S(=O)(=O)F)ccc1OCCOCCOCCOCCOCC(=O)O ; NCOCCOCCOCCOCCOc1ccc(/C=C/S(=O)(=O)F)cc1 ; O=C(COCCOCCOCCOCCOc1ccc(/C=C/S(=O)(=O)F)cc1)ON1C(=O)CCC1=O]

In another aspect, the present application provides a compound of Formular VI, or a pharmaceutically acceptable salt thereof:

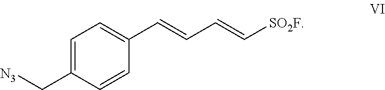 [SMILES: [N-]=[N+]=NCc1ccc(/C=C/C=C/S(=O)(=O)F)cc1]

In another aspect, the present application provides a pharmaceutical composition comprising the conjugate of the present application and a pharmaceutically acceptable carrier.

In another aspect, the present application provides a method for adjusting a tumor micro-environment of a subject, comprising administering to the subject the conjugate of the present application, or the pharmaceutical composition of the present application.

In another aspect, the present application provides a method for adjusting the immune reaction of a subject, comprising administering to the subject the conjugate of the present application, or the pharmaceutical composition of the present application.

In another aspect, the present application provides a method for preventing and/or treating disease in a subject in need of, comprising administering to the subject the conjugate of the present application, or the pharmaceutical composition of the present application.

In some embodiments, the disease comprises a tumor and/or an autoimmune disease.

In another aspect, the present application provides a diagnostic reagent comprising the conjugate of the present application.

In some embodiments, the diagnostic reagent is labeled.

In some embodiments, the label is selected from the group consisting of a radiolabel, a fluorophore, a chromophore, an imaging agent, and a metal ion.

Additional aspects and advantages of the present application will become readily apparent to those skilled in this art from the following detailed description, wherein only illustrative embodiments of the present application are shown and described. As will be realized, the present application is capable of other and different embodiments, and its several details are capable of modifications in various obvious respects, all without departing from the disclosure. Accordingly, the drawings and description are to be regarded as illustrative in nature, and not as restrictive.

### Incorporation by Reference

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWING

The novel features of the invention are set forth with particularity in the appended claims. A better understanding of the features and advantages of the present invention will be obtained by reference to the following detailed description that sets forth illustrative embodiments, in which the principles of the invention are employed, and the accompanying drawings (also “figure” and “FIG.” herein), of which:

FIG. 1 illustrates a reaction process for screening a candidate Michael acceptor as a linker.

FIG. 2 illustrates the chemical structure of the Michael acceptors candidates.

FIG. 3 illustrates the chemical structure of the modified Michael acceptor candidates.

FIG. 4 illustrates a reaction process for screening a modified candidate Michael acceptor as a linker.

FIG. 5 illustrates the chemical structure of the Michael acceptors candidates.

FIG. 6 illustrates a reaction process for screening a modified candidate Michael acceptor as a linker.

FIG. 7 illustrates the chemical structure of the previous reported linker.

FIG. 8 illustrates the reaction process of the reaction between the sfGFP E124C and the previous reported linker.

FIG. 9 illustrates the results of the reaction between the sfGFP E124C and the previous reported linker.

FIG. 10 illustrates the results of the competitivity experiments of the linker of present application and the previous reported linker.

FIGS. 11a-11c illustrate the stability of the linker of present application.

FIGS. 12a-12c illustrate the producing a conjugate with the linker of present application and verify the stability of the conjugate.

FIGS. 13a-13c illustrate the mass spectrometry result of a conjugate obtained by the linker of the present application showing stability in serum.

FIGS. 14a-14c illustrate the steps of producing a conjugate with the linker of present application.

FIGS. 15a-15c illustrate the mass spectrometry result of a conjugate with DAR 3.2 obtained by the linker of the present application.

FIGS. 16a-16b illustrate the mass spectrometry result of a conjugate with DAR 3.8 obtained by the linker of the present application.

FIGS. 17a-17h illustrates the results of the tumor cell killing test of a conjugate obtained by the linker of the present application.

FIG. 18 illustrates the LC-MS chromatograms and mass spectrum of SSF-PEG4-vc-PAB-MMAE 1.

FIG. 19 illustrates the LC-MS chromatograms of SSF-PEG4-GGFG-Dxd 3.

FIG. 20 illustrates the process of linker stability studies.

FIG. 21 illustrates the Hydrolytic stability results of MA 5 versus MA 2.

FIGS. 22A-22B illustrate the comparison of MA 2 with previously reported stable Cys-specific labeling reagents.

FIG. 23 illustrates the MS/MS spectra of GFP fragment modified with MA 2.

FIGS. 24A-24G illustrate the Cys-specific modification using SSF on different proteins.

FIGS. 25A-25D illustrate the result of anti-tumor activity of a conjugate obtained by the linker of the present application.

FIGS. 26A-26B illustrate the stability of MA 2 versus of maleimide in aqueous buffer.

FIGS. 27A-27H illustrate the preparation of a conjugate obtained by the linker of the present application comprising an SSF-ssDNA, and the application on single-cell sequencing thereof.

FIG. 28 illustrates the deconvoluted intact protein MS of a conjugate obtained by the linker of the present application.

FIG. 29 illustrates the deconvoluted intact protein MS of a conjugate obtained by the linker of the present application.

FIG. 30 illustrates results of Cell viability assays with cell lines (N87).

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

The term “conjugate” as used herein generally refers to a any substance formed from the joining together of separate parts. In the conjugate, the separate parts may be joined at one or more active site with each other. Moreover, the separate parts may be covalently or non-covalently associated with, or linked to, each other and exhibit various stoichiometric molar ratios. The conjugate may comprise peptides, polypeptides, proteins, prodrugs which are metabolized to an active agent in vivo, polymers, nucleic acid molecules, small molecules, binding agents, mimetic agents, synthetic drugs, inorganic molecules, organic molecules and radioisotopes. For example, the conjugate may comprise a drug and an antigen binding protein, and may be an antibody drug conjugate, ADC.

The term “ADC” as used herein generally refers to the linkage of an antigen binding protein with a drug. The linkage may be covalent bonds, or non-covalent interactions such as through electrostatic forces. Various linkers, may be employed in order to form the immunoconjugate. Additionally, the immunoconjugate may be provided in the form of a fusion protein that may be expressed from a polynucleotide encoding the immunoconjugate. As used herein, “fusion protein” refers to proteins created through the joining of two or more genes or gene fragments which originally coded for separate proteins (including peptides and polypeptides).

The term “biological macromolecule” as used herein generally refers to a biological molecule such as a nucleic acid, protein, antibody, carbohydrate, polysaccharide, lipid, and the lice.

The term “linker” as used herein generally refers to a chemical moiety or bond that attaches two or more molecules. The linker may be any molecule assembly capable of joining or connecting two or more scaffolds. The linker can be a molecule whose function is to act as a flexible linker between modules in a scaffold, or it can also be a molecule with additional function. In the present disclosure, the Linker may be used to link the fucose or fucose derivative to the active moiety. Linkers of different lengths allow one to attach the fucose or fucose derivative with different distances from the active moiety.

The term “functional molecule” as used herein generally refers to any molecule which is a component of the conjugate of the present application and can play a role in the function of the conjugate.

The term “biological function” as used herein generally refers to any activity or process carried out by the functional molecule of the present application in the biology. For example, the biological function may comprise any activity or process carried out by the functional molecule in vitro and/or in vivo, the biological function may comprise any activity or process carried out by the conjugate comprising the functional molecule in vitro and/or in vivo.

The term “functional group” as used herein generally refers to a group of the biological macromolecule which is capable of involving an addition reaction (for example, for a nucleophilic addition reaction). In the present application, the nucleophilic addition reaction may be a chemical addition reaction in which a nucleophile forms a sigma bond with an electron deficient species. The nucleophilic addition reaction may enable the conversion of carbonyl groups into a variety of functional groups. For example, the nucleophilic functional group of the biological macromolecule may be —SH, —NH₂, —SeH, —OH, or

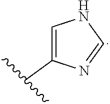 [SMILES: *c1c[nH]cn1]

The term “addition reaction” as used herein generally refers to an organic reaction where two or more molecules combine to form a larger one (the adduct). The additional reaction may comprise an electrophilic addition and a nucleophilic addition. The additional reaction may be limited to chemical compounds that have multiple bonds, such as molecules with carbon-carbon double bonds (alkenes), or with triple bonds (alkynes), and compounds that have rings, which are also considered points of unsaturation. For example, molecules comprising carbon-hetero double bonds like carbonyl (C═O) groups, or imine (C═N) groups may undergo the additional reaction.

The term “antigen binding protein” as used herein generally refers to a polypeptide molecule that specifically binds to an antigenic determinant. For example, the antigen binding protein may be directed to a target site, eg, an entity (eg, an effector moiety or a second antigen-binding moiety) that may be attached to a tumor stroma with a particular type of tumor cell or antigenic determinant. Is possible. Further, as defined herein, the antigen binding protein may comprise antibodies and fragments thereof. For example, the antigen binding protein may comprise an antibody antigen-binding domain comprising an antibody heavy chain variable region and an antibody light chain variable region. For example, the antigen binding protein may comprise an antibody constant region as further defined herein and known in the art. Useful heavy chain constant regions may comprise five isotypes: α, δ, ε, γ, γ, or μ. Useful light chain constant regions may comprise two isotypes: kappa and lambda. Useful light chain constant regions include either of two isotypes: x and k.

The term “antibody” as used herein generally refers to a polypeptide or a protein complex that specifically binds an epitope of an antigen or mimetope thereof Δn antibody includes an intact antibody, or a binding fragment thereof that competes with the intact antibody for specific binding and includes chimeric, humanized, fully human, and bispecific antibodies. Binding fragments include, but are not limited to, Fab, Fab′, F(ab′)2, Fv, and single-chain antibodies. In some embodiments, an antibody is referred to as an immunoglobulin and include the various classes and isotypes, such as IgA (IgA1 and IgA2), IgD, IgE, IgM, and IgG (IgG1, IgG3 and IgG4) etc. in some embodiments the term “antibody” as used herein refers to polyclonal and monoclonal antibodies and functional fragments thereof Δn antibody includes modified or derivatised antibody variants that retain the ability to specifically bind an epitope. Antibodies are capable of selectively binding to a target antigen or epitope. Antibodies may include, but are not limited to polyclonal antibodies, monoclonal antibodies (mAbs), humanized and other chimeric antibodies, single chain antibodies (scFvs), Fab fragments, F(ab′)2 fragments and disulfide-linked Fvs (sdFv) fragments. In some embodiments, the antibody is from any origin, such as mouse or human, including a chimeric antibody thereof. In some embodiments, the antibody is humanized.

The term “derivative” as used herein generally refers to a compound which is expected to exhibit a similar (for example, physical, or/and chemical, or/and biological) activity(ies) as that exhibited by the subject (parent) compound. For example, the derivative may be a precursor, a metabolite, a salt and/or an ester of the subject compound.

The term “drug” as used herein generally refers to any agent that is detrimental to the growth and proliferation of cells and may act to reduce, inhibit, or destroy a cell or malignancy. For example, the drug may comprise a toxin. For example, the drug may comprise a chemotherapy agent.

The term “cytokine” as used herein generally refers to a molecule that mediates and/or regulates a biological or cellular function or process (eg, immunity, inflammation and hematopoiesis). In present application, the cytokine may also comprise “lymphokines”, “chemokines”, “monokines” and “interleukins”. Examples of the cytokine may comprise GM-CSF, IL-1α, IL-1β, IL-2, IL-3, IL-4, IL-5, IL-6, IL-7, IL-8, IL-10, IL-12, IFN-α, IFN-β, IFN-γ, MIP-1α, MIP-1β, TGF-β, TNF-α, and TNF-β, but are not limited thereto. For example, the cytokine may comprise IL-2, IL-7, IL-10, IL-12, IL-15, IFN-α and IFN-γ. For example, the cytokine may be a human cytokine. The term “cytokine” as used herein also may refer to Sauve et al., Proc Natl Acad Sci USA 88, 4636-40 (1991); Hu et al., Blood 101, 4853-4861 (2003) and US patents. Application Publication No. 2003/0124678; Shanafelt et al., Nature Biotechnol 18, 1197-1202 (2000); Heaton et al., Cancer Res 53, 2597-602 (1993) and U.S. Pat. No. 5,229,109; Wild type cytokines such as IL-2 mutants described in US Patent Application Publication No. 2007/0036752; International Publication No. 2008/0034473; International Publication No. 2009/061853; PCT Patent Application PCT/EP2012/051991. Cytokine variants containing one or more amino acid mutations in the corresponding amino acid sequence are included. In addition, cytokine variants, such as IL-15 variants, are described herein. For example, the cytokine may be mutated to eliminate glycosylation.

The term “aryl” as used herein generally refers to a hydrocarbon ring system having a carbon atom having a hydrocarbon ring radical (i.e., a monocyclic hydrocarbon ring) or two to four fused rings, The cyclic hydrocarbon ring may be aromatic with 5 or 6 carbon atoms and each of the rings forming the hydrocarbon ring system may be aromatic and independently has 5 or 6 carbon atoms. For example, examples of the aryl groups may comprise phenyl, naphthalenyl (i.e., naphthyl) and anthracenyl. For example, the aryl may comprise preferably phenyl.

The term “alkyl” as used herein generally refers to at least one carbon atoms (For example, 1 to 20 carbon atoms. “1 to 20 carbon atoms” may refer to a straight chain and/or a branched group having an alkyl group of up to 1 carbon atom, 2 carbon atoms, 3 carbon atoms, etc., comprising up to 20 carbon atoms), And saturated aliphatic (i.e., non-aromatic) acyclic hydrocarbons (i.e., groups consisting of carbon atoms and hydrogen atoms) comprising a neighboring carbon-carbon double bond or carbon-carbon triple bond. For example, the alkyl may comprise from 1 to 10 carbon atoms. For example, the alkyl may comprise from 1 to 6 carbon atoms.

The term “drug” as used herein generally refers to any substance that alters the physiology of a subject. In the present application, the drug may comprise any compound having the desired biological activity and reactive functionalities available to prepare the conjugate of the present application. The desired biological activity may comprise an activity useful for diagnosing, curing, reducing, treating, or preventing a disease in a human or other animal. Thus, as long as they have the necessary reactive functional groups, the compounds may be associated with the term “drug” may be referred to in the official Chinese Pharmacopoeia, for example, in the official Homeopathic Pharmacopeia, or in the official National Formulary, or any of their amendments. Exemplary drugs may be described in the United States Physician's Desk Reference (PDR) and the Orange Book maintained by the US Food and Drug Administration (FDA). New drugs may constantly being discovered and developed, and the present application also incorporates those new drugs into the “drugs” of the drug conjugates of the present application.

The term “Toll-like receptor agonist” as used herein generally refers to any agonist of a Toll-like receptor. In the present application, the Toll-like receptor may be recognized by TLRs, which may activate immune cell responses. The TLRs may comprise TLR1, TLR2, TLR3, TLR4, TLR5, TLR6, TLR7, TLR8, TLR9, TLR10, TLR1 1, TLR12, and TLR13. For example, the Toll-like receptor agonist may comprise vaccine adjuvants in anti-tumor therapies for their ability to activate immune cells and promote inflammation.

The term “Sting agonist” as used herein generally refers to an agent capable of binding to STING and activating STING. For example, the activation of STING activity may comprise stimulation of inflammatory cytokines, comprising interferons, such as type 1 interferons, including IFN-a, IFN-b, type 3 interferons, for example, CXCL9, CCL4, CXCL11, CCL5, CCL3, or CCL8. STING agonist activity may also comprise stimulation of TANK binding kinase (TBK) 1 phosphorylation, interferon regulatory factor (IRF) activation (e.g, IRF3 activation), secretion of interferon-y-inducible protein (IP-10), or other inflammatory proteins and cytokines. STING Agonist activity may be determined, for example, by the ability of a compound to stimulate activation of the STING pathway as detected using an interferon stimulation assay, a reporter gene assay (e.g., a hSTING wt assay, or a THP-1 Dual assay), a TBK1 activation assay, IP-10 assay, or other assays known to persons skilled in the art. STING Agonist activity may also be determined by the ability of a compound to increase the level of transcription of genes that encode proteins activated by STING or the STING pathway. Such activity may be detected, for example, using an RNAseq assay.

The term “pharmaceutically acceptable carrier” as used herein generally refers to a non-API (API means a pharmaceutically active ingredient) such as disintegrants, binders, fillers, and lubricants used to form a pharmaceutical product. The pharmaceutically acceptable carriers may conform to established government standards, including standards promulgated by the US Food and Drug Administration and the European Food and Drug Administration, and are generally safe for human administration. For example, the pharmaceutically acceptable carrier may comprise a sterile aqueous or non-aqueous solution, a dispersion, a suspension, a emulsion, and/or a sterile injectable solution or dispersion just prior to use.

The term “tumor” generally refers to a malignancy characterized by deregulated or uncontrolled cell growth. For example, the tumor may comprise primary malignant tumors (e.g., those whose cells have not migrated to sites in the subject's body other than the site of the original tumor) and secondary malignant tumors (e.g., those arising from metastasis, the migration of tumor cells to secondary sites that are different from the site of the original tumor). The tumor may comprise a solid tumor, and/or a non-solid tumor.

The term “tumor micro-environment” generally refers to a complex surrounding microenvironment of tumor cells. For example, the tumor micro-environment may comprise surrounding blood vessels, immune cells, fibroblasts, bone marrow-derived inflammatory cells, various signal molecules and/or extracellular matrix (ECM). For example, the tumor micro-environment may harbor cancer stem cells and other molecules that contribute to tumor development and progression. Consequently, targeting and manipulating the cells and factors in the tumor micro-environment during treatment may help control malignancies and achieve positive health outcomes.

The term “immune reaction” generally refers to the subject's defense against foreign substances and/or pathogens. The immune reaction may lead to immune response, for example, the recognition and binding of an antigen by its specific antibody or by a previously sensitized lymphocyte.

The term “autoimmune disease” generally refers to any disease and/or disorder induced by an immune-mediated attack to the subject's own organs. Examples of the autoimmune disease may comprise Rheumatoid arthritis, Systemic lupus erythematosus (lupus), Inflammatory bowel disease (IBD), Multiple sclerosis (MS), Type 1 diabetes mellitus, Guillain-Barre syndrome, Chronic inflammatory demyelinating polyneuropathy, Psoriasis, Graves' disease, Hashimoto's thyroiditis, Myasthenia gravis, and/or Vasculitis.

The term “treating” as used herein generally refers to ameliorating a disease or disorder (i.e., slowing or arresting or reducing the development of the disease (for example, the tumor) or at least one of the clinical symptoms thereof. For example, the treating may comprise alleviating or ameliorating at least one physical parameter comprising those which may not be discernible by the patient.

The term “preventing” as used herein generally refers to a prophylactic treatment of a disease or disorder; or delaying the onset or progression of a disease or disorder

As used herein, the term “a”, “an”, “the” and similar terms used in the context of the present application (especially in the context of the claims) are to be construed to cover both the singular and plural unless otherwise indicated herein or clearly contradicted by the context.

### Conjugate

In one aspect, the present application provides a conjugate of formula 1, M-[(L1)ₐ-(L2)_{b}-(D)_{c}]
1, wherein L1 is a compound of formula 1,

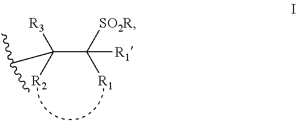

R is —F or —OH, wherein M is a biological macromolecule, and M is linked to L1 with a nucleophilic functional group of M, L2 is a linker, and L2 is linked to R₁, R₃ or R₂, D is a functional molecule, a is an integer of 1 to 10, b, c is each independently an integer of 0 to 10, provided that b and c are not simultaneously 0, wherein R₁ is H, an optionally substituted alkyl or an optionally substituted aryl, wherein R₁′ is H or the isotope thereof, wherein R₂ is H, an optionally substituted alkyl or an optionally substituted aryl, wherein R₃ is H, an optionally substituted alkyl or an optionally substituted aryl, optionally, a C linking R₁ and a C linking R₂ form a ring.

For example, the nucleophilic functional group of M may be selected from a group consisting of —SH, —NH₂, —SeH, —OH, and

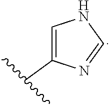 [SMILES: *c1c[nH]cn1]

In another aspect, the present application provides a conjugate of formula 2, M-S-[(L1)ₐ-(L2)_{b}-(D)_{c}] 2, wherein M-S is a biological macromolecule with a cysteine, M-S is linked to L1 with the cysteine, L1 is a compound of formula 1,

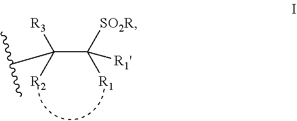

R is —F or —OH, L2 is a linker, L2 is linked to R₁, R₃ or R₂, D is a functional molecule, a is an integer of 1 to 10, b, c is each independently an integer of 0 to 10, provided that b and c are not simultaneously 0, wherein R₁ is H, an optionally substituted alkyl or an optionally substituted Aryl, wherein R₁′ is H or the isotope thereof, wherein R₂ is H, an optionally substituted alkyl or an optionally substituted Aryl, wherein R₃ is H, an optionally substituted alkyl or an optionally substituted Aryl, optionally, a C linking R₁ and a C linking R₂ form a ring.

For example, M may be selected from a group consisting of a protein, a DNA, a RNA, and a virus.

For example, M may be a biological macromolecule expressed on the surface of a cell.

For example, M may be an antigen binding protein or a fragment thereof.

For example, M may be a monoclonal antibody, a chimeric antibody, a humanized antibody, a human engineered antibody, a human antibody, a single chain antibody(scFv) or an antibody fragment.

For example, M may comprise a functional group for an addition reaction.

For example, L2 may be selected from the group consisting of a cleavable linker, a non-cleavable linker, a hydrophilic linker, a hydrophobic linker, a procharged linker, an uncharged linker and a dicarboxylic acid-based linker.

For example, L2 may be selected from the group consisting of VC-PAB, N-succinimidyl-3-(2-pyridyldithio)propionate (SPDP), N-succinimidyl 4-(2-pyridyldithio)pentanoate (SPP), N-succinimidyl 4-(2-pyridyldithio)butanoate (SPDB), N-succinimidyl-4-(2-pyridyldithio)-2-sulfo-butanoate (sulfo-SPDB), N-succinimidyl iodoacetate (SIA), N-succinimidyl(4-iodoacetyl)aminobenzoate (SIAB), maleimide PEG NHS, N-succinimidyl 4-(maleimidomethyl) cyclohexanecarboxylate (SMCC), N-sulfosuccinimidyl 4-(maleimidomethyl) cyclohexanecarboxylate (sulfo-SMCC) or 2,5-dioxopyrrolidin-1-yl 17-(2,5-dioxo-2,5-dihydro-1H-pyrrol-1-yl)-5,8,11,14-tetraoxo-4,7,10,13-tetraazaheptadecan-1-oate (CX1-1).

For example, D may have a biological function.

For example, D and/or a derivative thereof may be capable of inhibiting the growth of a tumor cell.

For example, D may be a drug.

For example, D may be selected from the group consisting of a V-ATPase inhibitor, a pro-apoptotic agent, a Bcl2 inhibitor, an MCL1 inhibitor, a HSP90 inhibitor, an IAP inhibitor, an mTor inhibitor, a microtubule stabilizer, a microtubule destabilizer, an auristatin, a dolastatin, a maytansinoid, a MetAP (methionine aminopeptidase), an inhibitor of nuclear export of proteins CRM1, a DPPIV inhibitor, proteasome inhibitors, inhibitors of phosphoryl transfer reactions in mitochondria, a protein synthesis inhibitor, a kinase inhibitor, a CDK2 inhibitor, a CDK9 inhibitor, a kinesin inhibitor, an HDAC inhibitor, a DNA damaging agent, a DNA alkylating agent, a DNA intercalator, a DNA minor groove binder,a DHFR inhibitor, a nucleoside analog, a HD AC inhibitor; an anthracycline; a NAMPT inhibitor; a hydrophilic prodrug; a SN-38 glucuronide, a etoposide phosphate; a nitrogen mustard, a proteosome inhibitor, a cytokine, a Toll-like receptor agonist and a Sting agonist.

For example, D may be MMAE or a derivative thereof; melphalan or a derivative thereof; lenalidomide or a derivative thereof; IL-2 or a derivative thereof; neoleukin-2/15 or a derivative thereof; T785 or a derivative thereof; or, MSA-2 or a derivative thereof.

For example, R₁′ may be —H. For example, R₁ is —H. For example, R₃ is —H.

For example, R₂ may be an optionally substituted phenyl.

For example, R₂ may be R_{4 l},

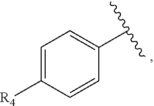

wherein R₄ may be selected from a group consisting of —OH, —PO₃H₂, —SeH, —SH, optionally substituted alkyl-OH, optionally substituted alkyl-halogen, optionally substituted alkyl-N₃, —B(OH)₂, -halogen, —OTf, optionally substituted alkyl-NH₂, —O-optionally substituted alkyl-C≡CH, —CO—NH—C≡CH-optionally substituted alkyl.

For example, R₂ may be

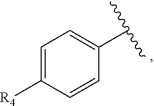

wherein R₄ may be selected from a group consisting of: —OH, —PO₃H₂, —SeH, —SH, —CH₂OH, —CH₂Br, —CH₂N₃, —B(OH)₂, —Br, —OTf, —CH₂NH₂, —Cl, —OCH₂C≡CH or —CO—NH—C≡CH.

For example, R₂ may be

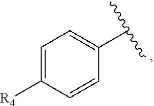

wherein R₄ may be —O—(CH₂)ₙ₁—COO—R₅, n₁ may be an integer of 1 to 10, wherein R₅ may be selected from a group consisting of:

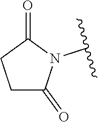 [SMILES: *N1C(=O)CCC1=O]

### and H.

For example, R₂ may be

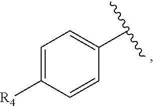

wherein R₄ may be —O—(CH₂)ₙ₂—CO—NH—R₆, n₂ may be an integer of 1 to 10, wherein R₆ may be —(CH₂)ₙ₃—CO—R₇, n₃ may be an integer of 1 to 10, wherein R₇ may be selected from a group consisting of:

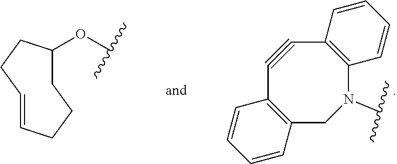 [SMILES: *OC1CC/C=C/CCC1 ; *N1Cc2ccccc2C#Cc2ccccc21]

For example, R₂ may be

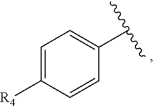

wherein R₄ may be —O—(CH₂)ₙ₂—CO—NH—R₆, n₂ may be an integer of 1 to 10, wherein R₆ may be —(CH₂)ₙ₄-R₈, n₄ may be an integer of 1 to 10, wherein R₈ may be selected from a group consisting of:

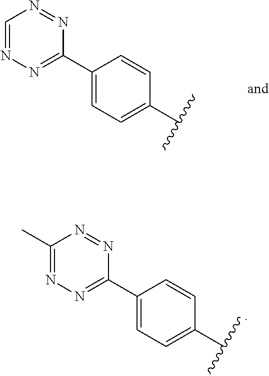 [SMILES: *c1ccc(-c2nncnn2)cc1 ; *c1ccc(-c2nnc(C)nn2)cc1]

For example, R₂ may be

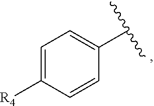

wherein R₄ may be —O—(CH₂)ₙ₂—CO—NH—R₆, n₂ may be an integer of 1 to 10, wherein R₆ may be —(CH₂CH₂—O)ₙ₅—(CH₂)ₙ₆—NH—CO—O—R₉, n₈ may be an integer of 1 to 10, n₆ may be an integer of 1 to 10, wherein R₉ may be selected from a group consisting of: H and

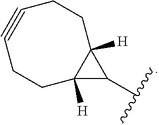 [SMILES: *C1[C@H]2CCC#CCC[C@@H]12]

For example, R₂ may be

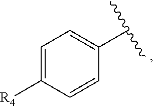

wherein R₄ may be —(OCH₂CH₂)ₙ₇—O—(CH₂)ₙ₈—R₁₀, n₇ may be an integer of 1 to 10, n₈ may be an integer of 1 to 10, wherein R₁₀ may be selected from a group consisting of: —COOH, —NH₂ and

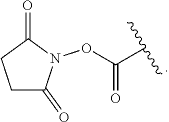 [SMILES: *C(=O)ON1C(=O)CCC1=O]

For example, R₂ may be

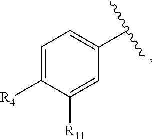

wherein R₄ may be —(OCH₂CH₂)ₙ₇—O—(CH₂)ₙ₈—R₁₀, n₇ is an integer of 1 to 10, n₈ is an integer of 1 to 10, wherein R₁₀ may be selected from a group consisting of: —COOH, —NH₂ and

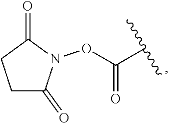 [SMILES: *C(=O)ON1C(=O)CCC1=O]

wherein R₁ may be selected from a group consisting of: optionally substituted alkyl-halogen, optionally substituted alkyl-N and 0-optionally substituted alkyl.

For example, R₂ may be

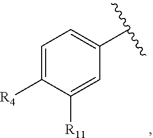

wherein R₄ may be —(OCH₂CH₂)ₙ₇—O—(CH₂)ₙ₈—R₁₀, n₇ may be an integer of 1 to 10, n₈ is an integer of 1 to 10, wherein R₁₀ may be selected from a group consisting of: —COOH, —NH₂ and

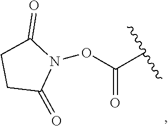 [SMILES: *C(=O)ON1C(=O)CCC1=O]

wherein R₁ may be selected from a group consisting of: —CF₃, —CN and —OCH₃. For example, R₂ may be optionally substituted alkyl-CH═CH—R₁₂, wherein R₁₂ may be

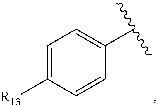

wherein R₁₃ may be —CH₂N₃.

For example, the ring may be an optionally substituted cycloolefin, or an optionally substituted aryl-cycloolefin.

For example, the ring may be selected from the group consisting of:

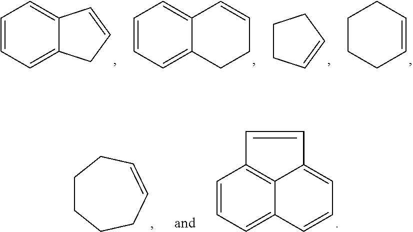

For example, L1 may be selected from the group consisting of:

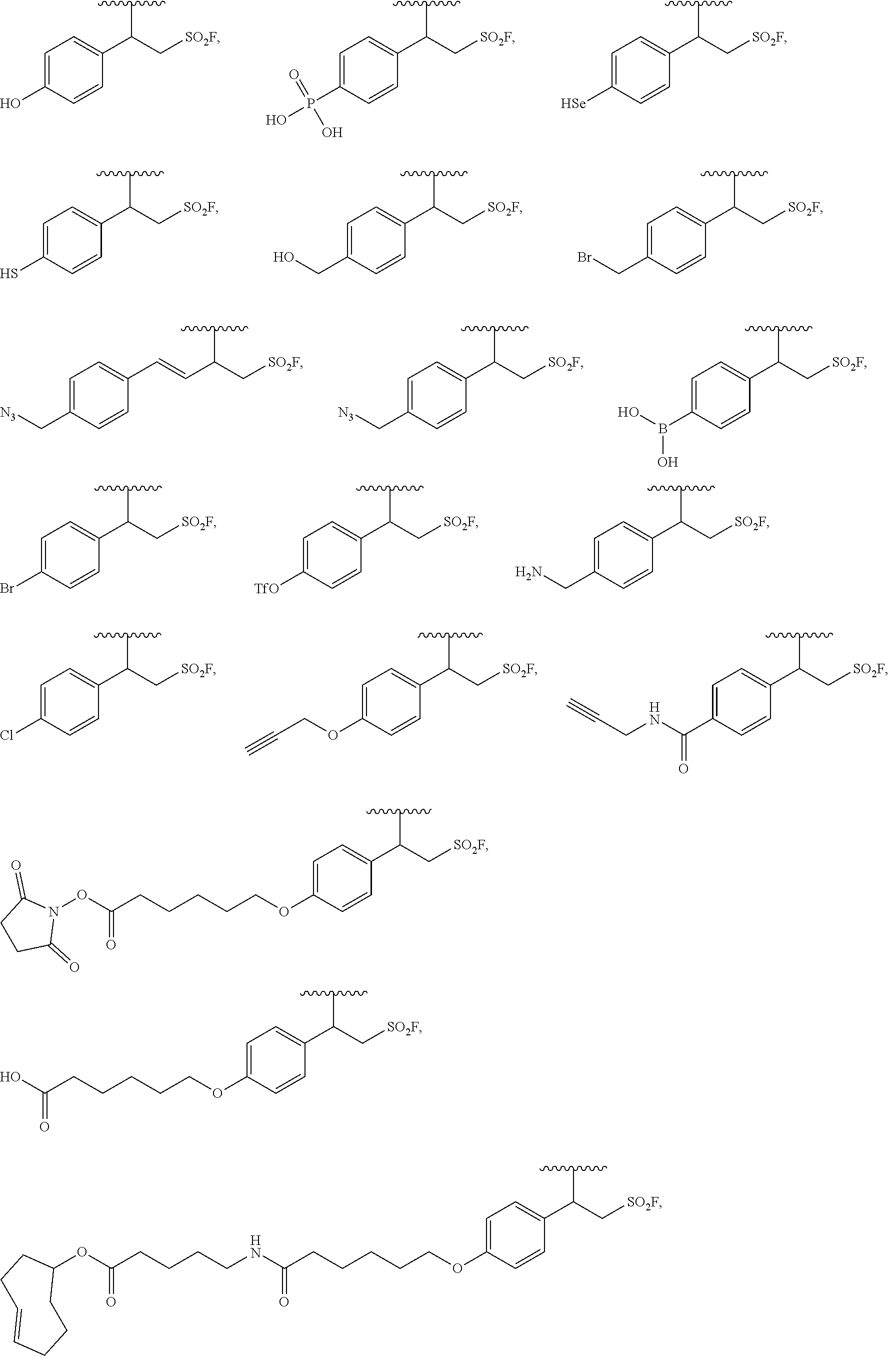

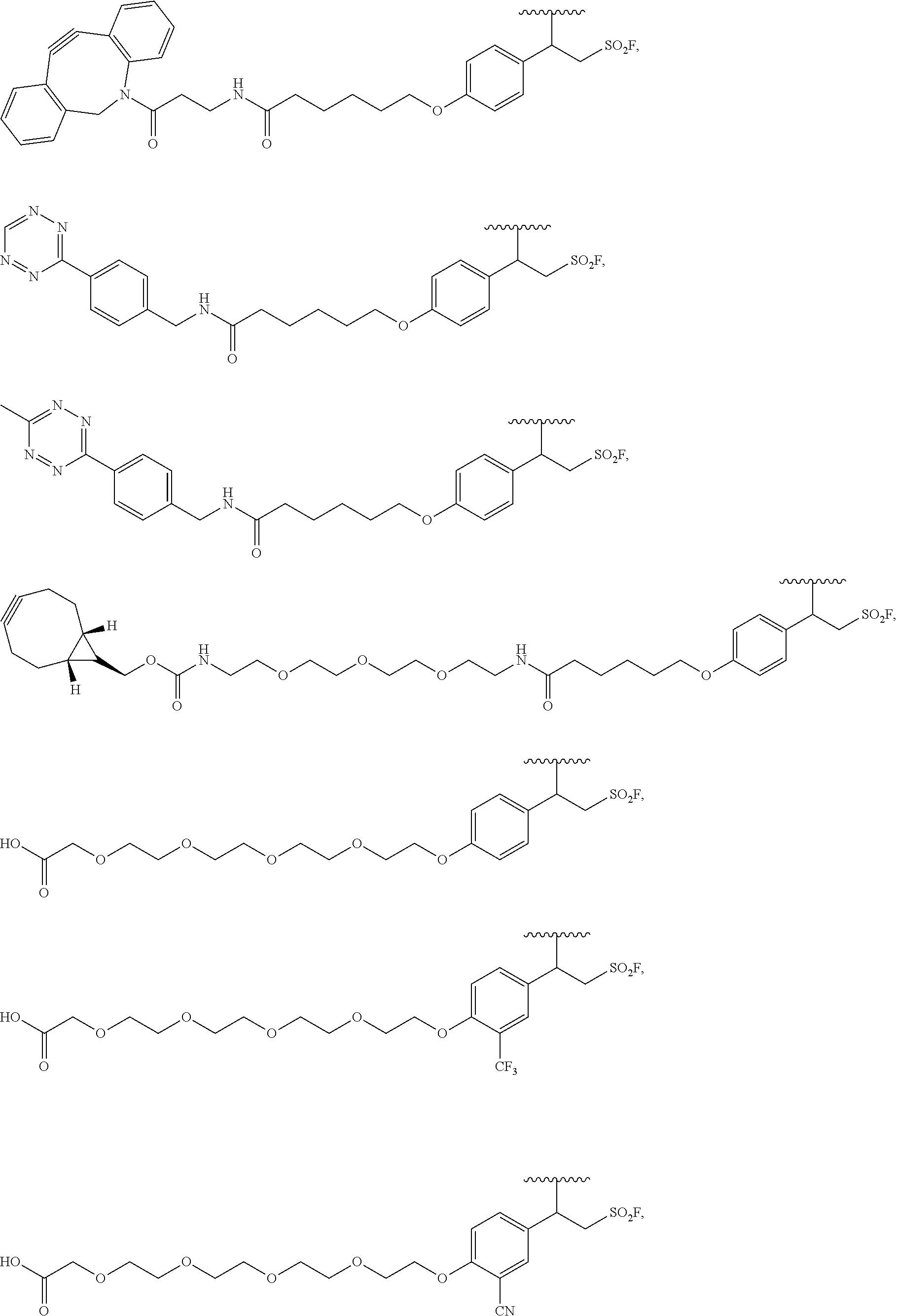

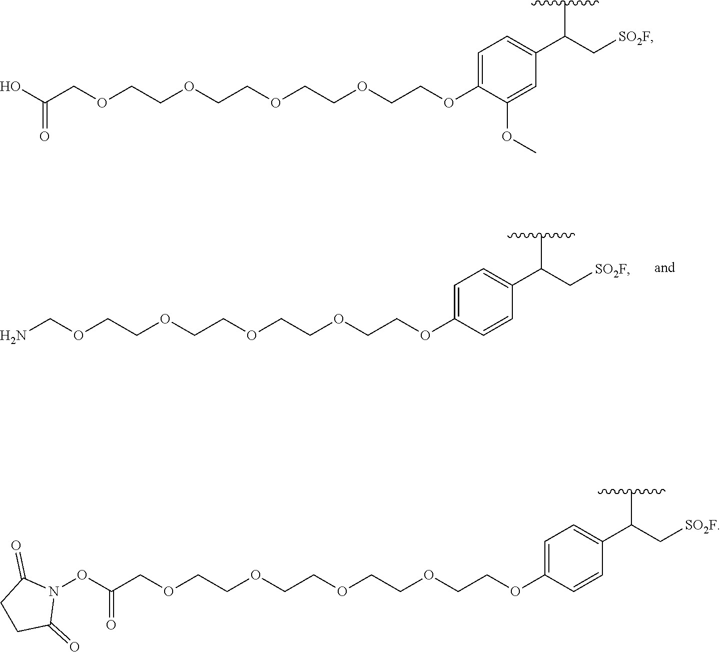 [SMILES: *C(CS(=O)(=O)F)c1ccc(OCCOCCOCCOCCOCC(=O)O)c(OC)c1 ; *C(CS(=O)(=O)F)c1ccc(OCCOCCOCCOCCOCN)cc1 ; *C(CS(=O)(=O)F)c1ccc(OCCOCCOCCOCCOCC(=O)ON2C(=O)CCC2=O)cc1]

For example, the conjugate may be selected from the group consisting of:

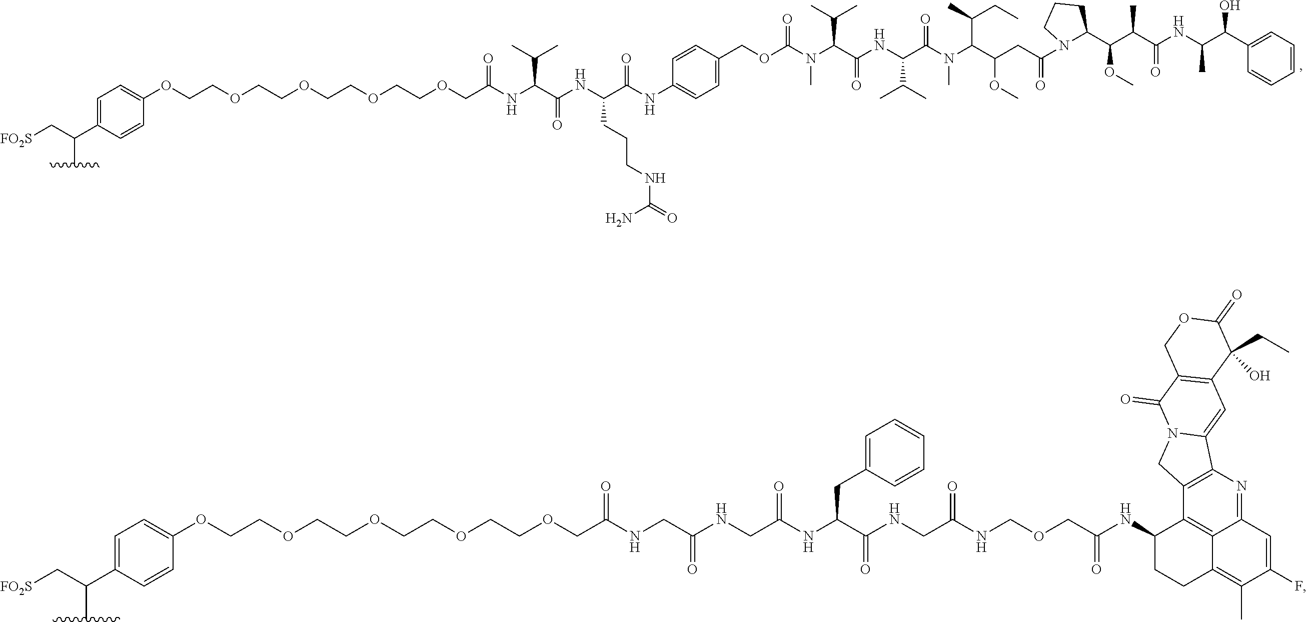

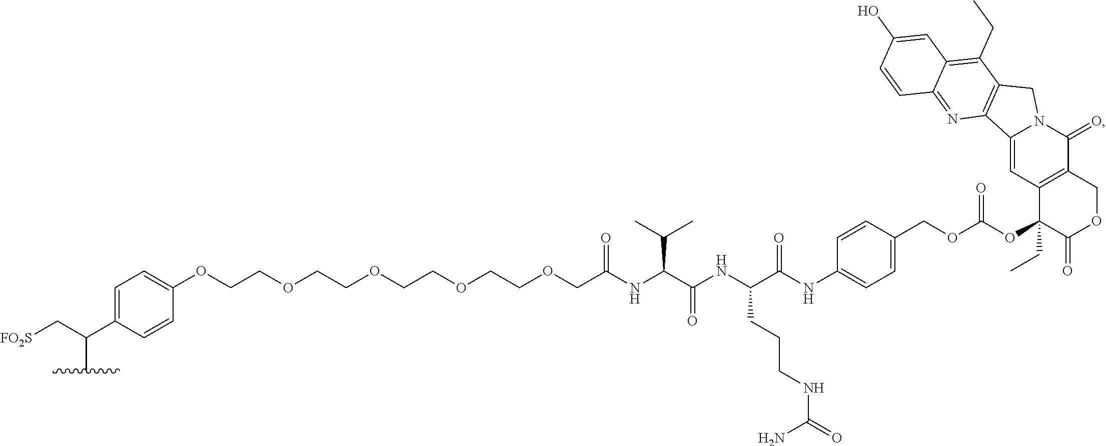

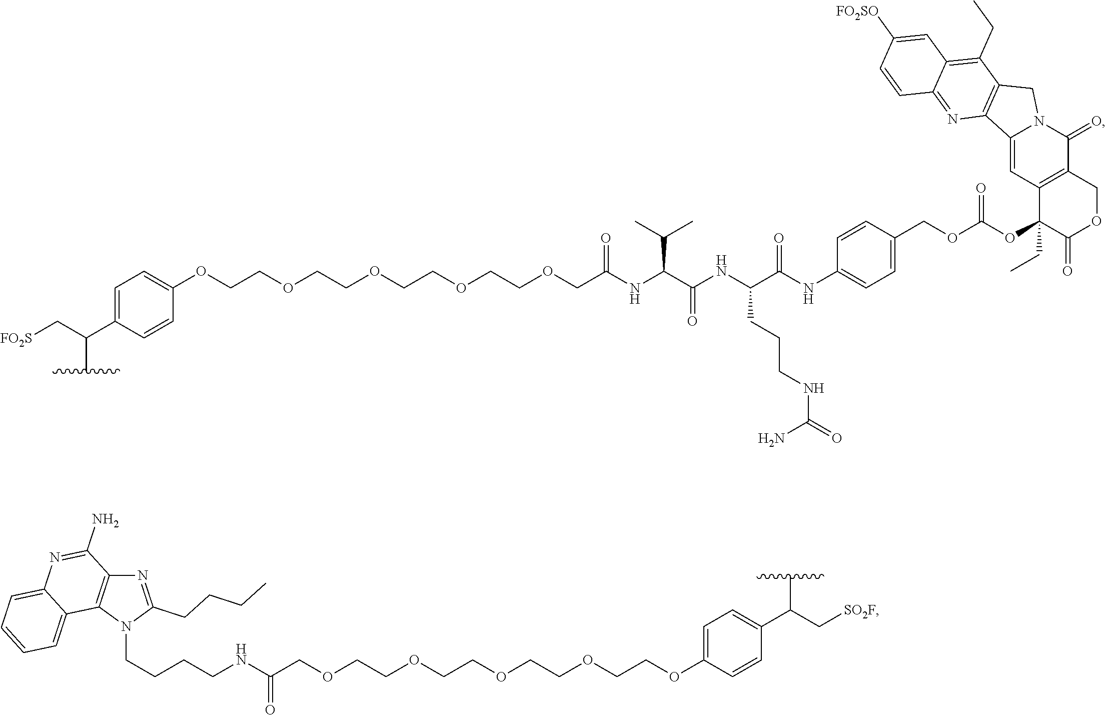

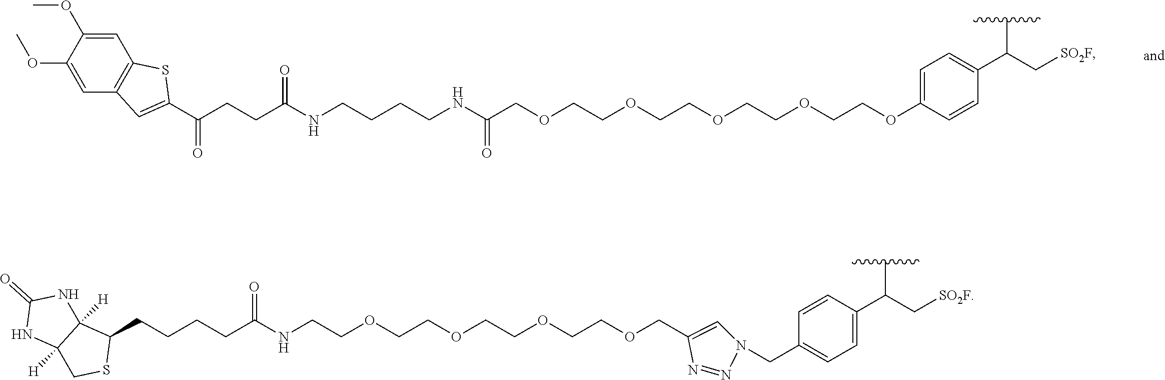 [SMILES: *C(CS(=O)(=O)F)c1ccc(OCCOCCOCCOCCOCC(=O)NCCCCNC(=O)CCC(=O)c2cc3cc(OC)c(OC)cc3s2)cc1 ; *C(CS(=O)(=O)F)c1ccc(Cn2cc(COCCOCCOCCOCCNC(=O)CCCC[C@H]3SC[C@H]4NC(=O)N[C@H]43)nn2)cc1]

For example, the conjugate of formula 1 or formula 2 may be as following:

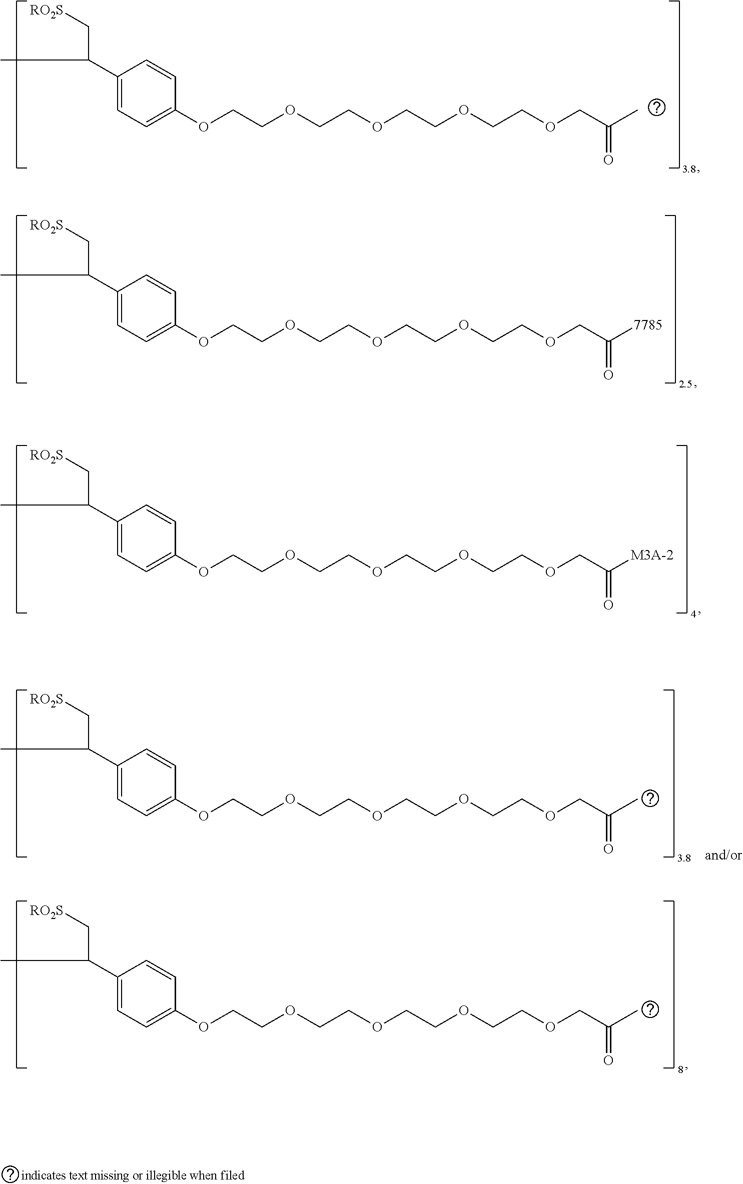

wherein R is —F or —OH.

In another aspect, the present application provides a conjugate of formula 3, (L1)ₐ-(L2)_{b}-(D)_{c} 3, wherein L1 is a compound of formula III

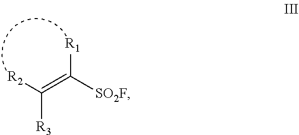

L2 is a linker, and L2 is linked to R₁, R₃ or R₂, D is a functional molecule, a is an integer of 1 to 10, b, c is each independently an integer of 0 to 10, provided that b and c are not simultaneously 0, wherein R₁ is H, an optionally substituted alkyl or an optionally substituted aryl, wherein R₂ is H, an optionally substituted alkyl or an optionally substituted aryl, wherein R₃ is H, an optionally substituted alkyl or an optionally substituted aryl, optionally, a C linking R₁ and a C linking R₂ form a ring.

In some embodiments, in the conjugate of formula 3, L2 is selected from the group consisting of a cleavable linker, a non-cleavable linker, a hydrophilic linker, a hydrophobic linker, a procharged linker, an uncharged linker and a dicarboxylic acid-based linker.

For example, L2 may be selected from the group consisting of VC-PAB, N-succinimidyl-3-(2-pyridyldithio)propionate (SPDP), N-succinimidyl 4-(2-pyridyldithio)pentanoate (SPP), N-succinimidyl 4-(2-pyridyldithio)butanoate (SPDB), N-succinimidyl-4-(2-pyridyldithio)-2-sulfo-butanoate (sulfo-SPDB), N-succinimidyl iodoacetate (SIA), N-succinimidyl(4-iodoacetyl)aminobenzoate (SIAB), maleimide PEG NHS, N-succinimidyl 4-(maleimidomethyl) cyclohexanecarboxylate (SMCC), N-sulfosuccinimidyl 4-(maleimidomethyl) cyclohexanecarboxylate (sulfo-SMCC) or 2,5-dioxopyrrolidin-1-yl 17-(2,5-dioxo-2,5-dihydro-1H-pyrrol-1-yl)-5,8,11,14-tetraoxo-4,7,10,13-tetraazaheptadecan-1-oate (CX1-1).

For example, D may have a biological function. For example, D and/or a derivative thereof may be capable of inhibiting the growth of a tumor cell. For example, D may be a drug.

For example, D may be selected from the group consisting of a V-ATPase inhibitor, a pro-apoptotic agent, a Bcl2 inhibitor, an MCL1 inhibitor, a HSP90 inhibitor, an IAP inhibitor, an mTor inhibitor, a microtubule stabilizer, a microtubule destabilizer, an auristatin, a dolastatin, a maytansinoid, a MetAP (methionine aminopeptidase), an inhibitor of nuclear export of proteins CRM1, a DPPIV inhibitor, proteasome inhibitors, inhibitors of phosphoryl transfer reactions in mitochondria, a protein synthesis inhibitor, a kinase inhibitor, a CDK2 inhibitor, a CDK9 inhibitor, a kinesin inhibitor, an HDAC inhibitor, a DNA damaging agent, a DNA alkylating agent, a DNA intercalator, a DNA minor groove binder, a DHFR inhibitor, a nucleoside analog, a HD AC inhibitor; an anthracycline; a NAMPT inhibitor; a hydrophilic prodrug; a SN-38 glucuronide, a etoposide phosphate; a nitrogen mustard, a proteosome inhibitor, a cytokine, a Toll-like receptor agonist and a Sting agonist. For example, D may be MMAE or a derivative thereof; melphalan or a derivative thereof; lenalidomide or a derivative thereof; IL-2 or a derivative thereof; neoleukin-2/15 or a derivative thereof; T785 or a derivative thereof; or, MSA-2 or a derivative thereof.

For example, R₁ may be —H.

For example, R₃ may be —H.

For example, R₂ may be an optionally substituted phenyl.

For example, R₂ may be

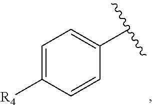

wherein R₄ may be selected from a group consisting of —OH, —PO₃H₂, —SeH, —SH, optionally substituted alkyl-OH, optionally substituted alkyl-halogen, optionally substituted alkyl-N₃, —B(OH)₂, -halogen, —OTf, optionally substituted alkyl-NH₂, —O-optionally substituted alkyl-C≡CH, —CO—NH—C≡CH— optionally substituted alkyl.

For example, R₂ may be

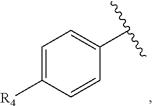

wherein R₄ may be selected from a group consisting of —OH, —PO₃H₂, —SeH, —SH, —CH₂OH, —CH₂Br, —CH₂N₃, —B(OH)₂, —Br, —OTf, —CH₂NH₂, —Cl, —OCH₂C≡CH or —CO—NH—C≡CH.

For example, R₂ may be

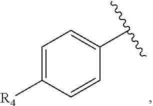

wherein R₄ may be —O—(CH₂)ₙ₁—COO—R₅, n₁ is an integer of 1 to 10, wherein R₅ may be selected from a group consisting of:

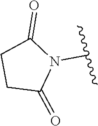 [SMILES: *N1C(=O)CCC1=O]

### and —H.

For example, R₂ may be

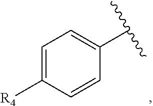

wherein R₄ may be —O—(CH₂)ₙ₂—CO—NH—R₆, n₂ may be an integer of 1 to 10, wherein R₆ may be —(CH₂)ₙ₃—CO—R₇, n₃ may be an integer of 1 to 10, wherein R₇ may be selected from a group consisting of;

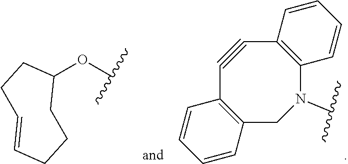 [SMILES: *OC1CC/C=C/CCC1 ; *N1Cc2ccccc2C#Cc2ccccc21]

For example, R₂ may be

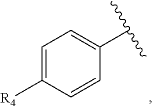

wherein R₄ may be —O—(CH₂)ₙ₂—CO—NH—R₆, n₂ is an integer of 1 to 10, wherein R₆ may be —(CH₂)ₙ₄-R₈, n₄ may be an integer of 1 to 10, wherein R₈ may be selected from a group consisting of:

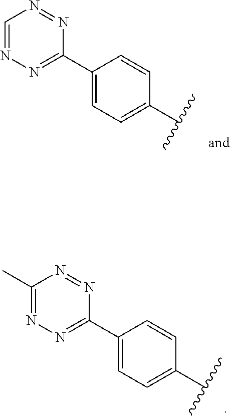 [SMILES: *c1ccc(-c2nncnn2)cc1 ; *c1ccc(-c2nnc(C)nn2)cc1]

For example, R₂ may be

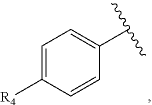

wherein R₄ may be —O—(CH₂)ₙ₂—CO—NH—R₆, n₂ is an integer of 1 to 10, wherein R₆ may be —(CH₂CH₂—O)ₙ₅—(CH₂)ₙ₆—NH—CO—O—R₉, n₈ may be an integer of 1 to 10, n₆ may be an integer of 1 to 10, wherein R₉ may be selected from a group consisting of: H and

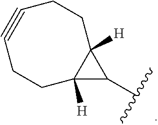 [SMILES: *C1[C@H]2CCC#CCC[C@@H]12]

For example, R₂ may be

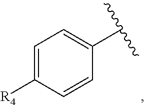

wherein R₄ may be —(OCH₂CH₂)ₙ₇—O—(CH₂)ₙ₈—R₁₀, n₇ may be an integer of 1 to 10, n₈ may be an integer of 1 to 10, wherein R₁₀ may be selected from a group consisting of: —COOH, —NH₂ and

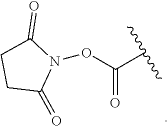 [SMILES: *C(=O)ON1C(=O)CCC1=O]

For example, R₂ may be

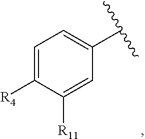

wherein R₄ may be —(OCH₂CH₂)ₙ₇—O—(CH₂)ₙ₈—R₁₀, n₇ may be an integer of 1 to 10, n₈ may be an integer of 1 to 10, wherein R₁₀ may be selected from a group consisting of: —COOH, —NH₂ and

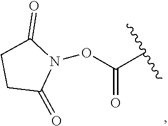 [SMILES: *C(=O)ON1C(=O)CCC1=O]

wherein R₁ may be selected from a group consisting of: optionally substituted alkyl-halogen, optionally substituted alkyl-N and 0-optionally substituted alkyl.

For example, R₂ may be

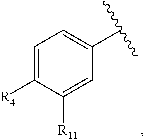

wherein R₄ may be —(OCH₂CH₂)ₙ₇—O—(CH₂)ₙ₈—R₁₀, n₇ may be an integer of 1 to 10, n₈ may be an integer of 1 to 10, wherein R₁₀ may be selected from a group consisting of: —COOH, —NH₂ and

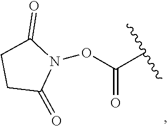 [SMILES: *C(=O)ON1C(=O)CCC1=O]

wherein R₁ may be selected from a group consisting of: —CF₃, —CN and —OCH₃.

For example, R₂ may be optionally substituted alkyl-CH═CH—R₁₂, wherein R₁₂ may be

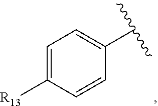

wherein R₁₃ may be —CH₂N₃.

For example, said ring may be an optionally substituted cycloolefin, or an optionally substituted aryl-cycloolefin.

For example, said ring may be selected from the group consisting of:

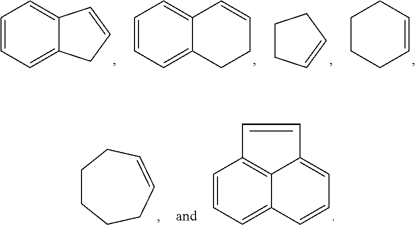

For example, said L1 may be selected from the group consisting of:

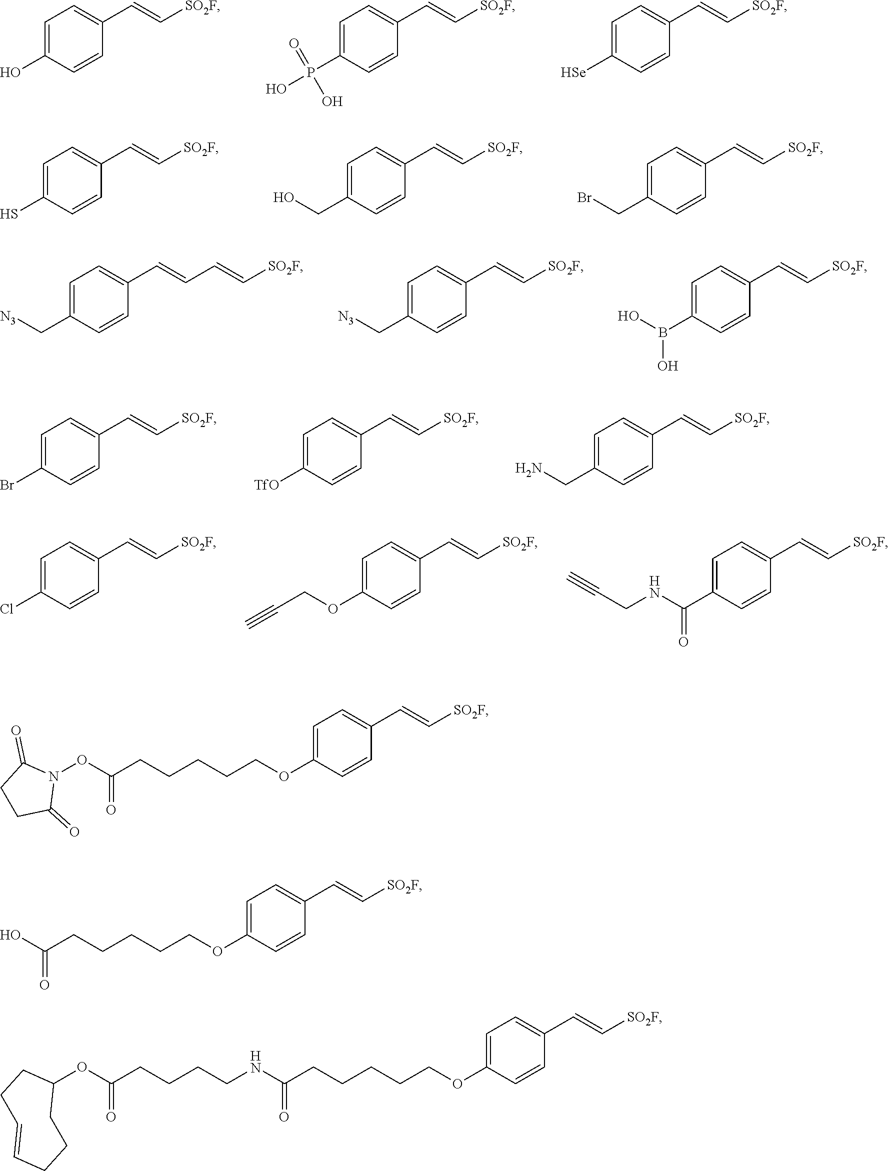

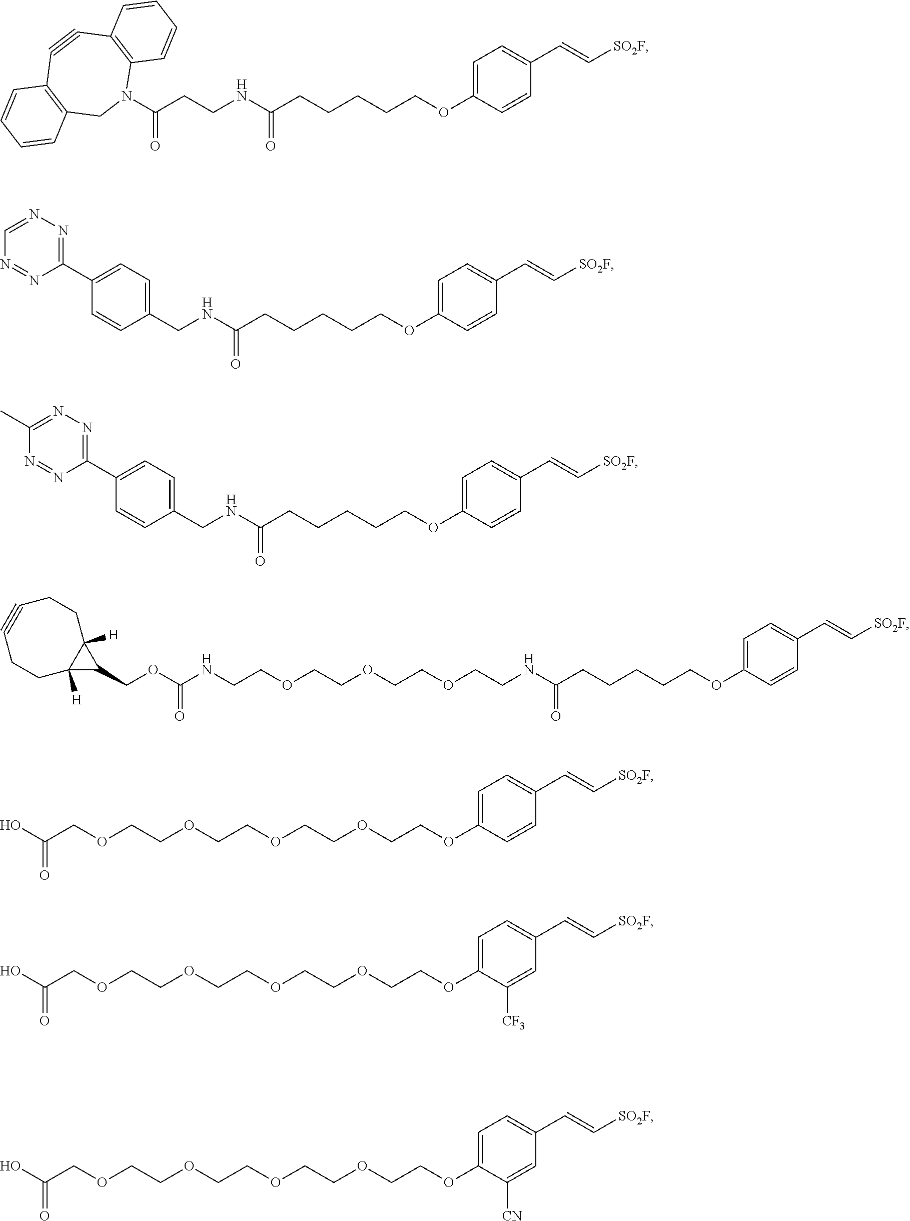

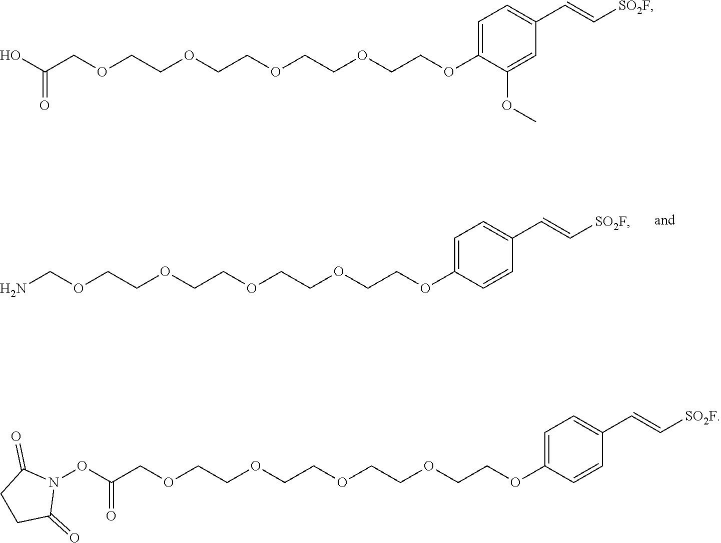 [SMILES: COc1cc(/C=C/S(=O)(=O)F)ccc1OCCOCCOCCOCCOCC(=O)O ; NCOCCOCCOCCOCCOc1ccc(/C=C/S(=O)(=O)F)cc1 ; O=C(COCCOCCOCCOCCOc1ccc(/C=C/S(=O)(=O)F)cc1)ON1C(=O)CCC1=O]

For example, said conjugate may be selected from the group consisting of.

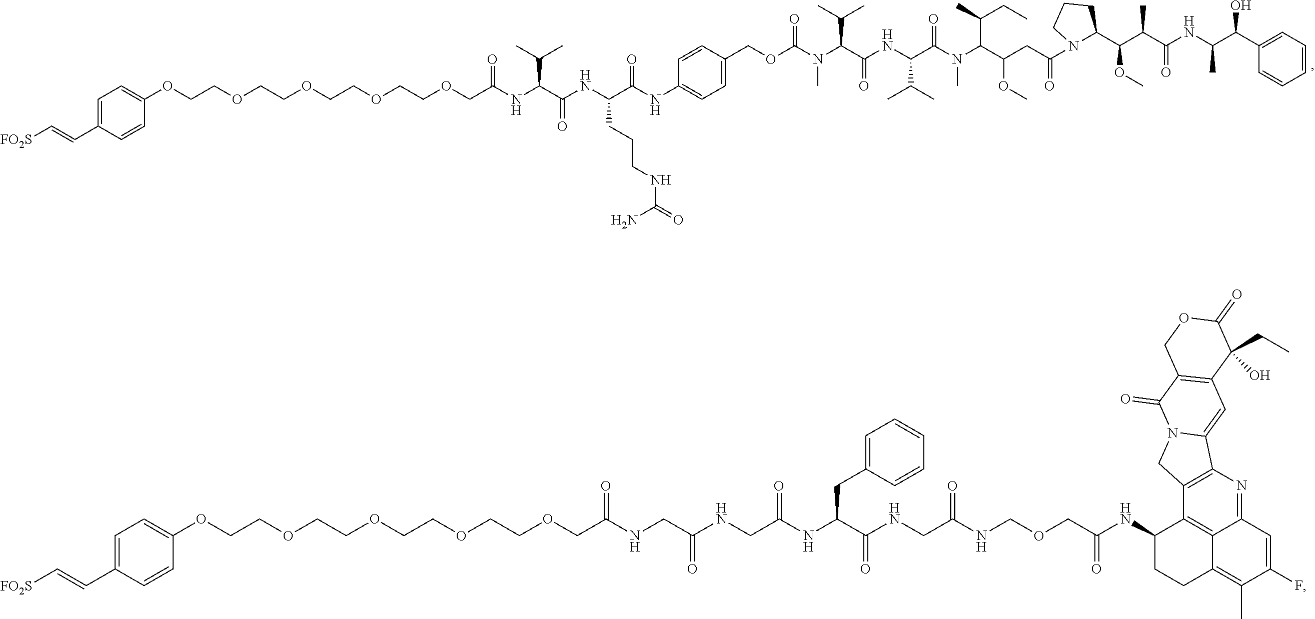

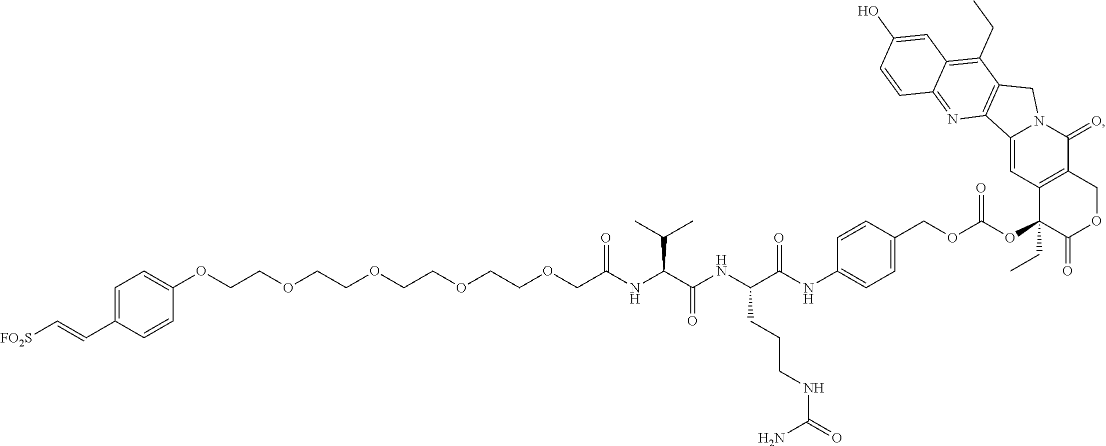

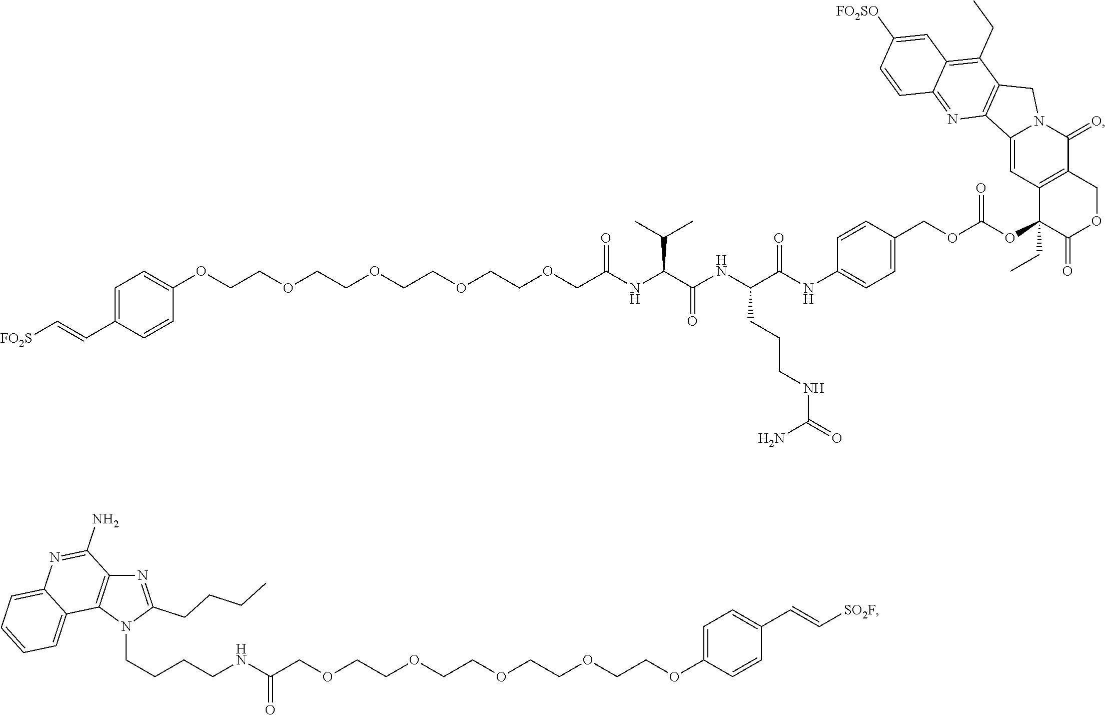

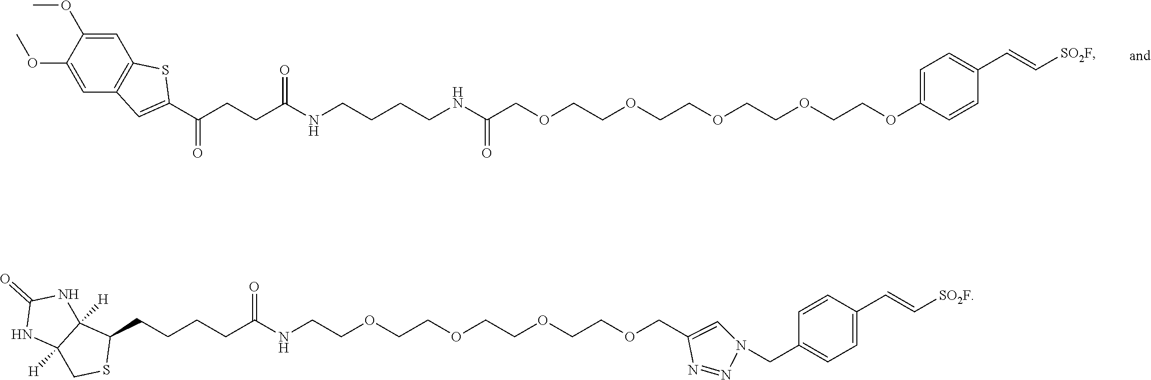 [SMILES: COc1cc2cc(C(=O)CCC(=O)NCCCCNC(=O)COCCOCCOCCOCCOc3ccc(/C=C/S(=O)(=O)F)cc3)sc2cc1OC ; O=C(CCCC[C@H]1SC[C@H]2NC(=O)N[C@H]21)NCCOCCOCCOCCOCc1cn(Cc2ccc(/C=C/S(=O)(=O)F)cc2)nn1]

### Method

In another aspect, the present application provides a method for preparing a conjugate, comprising the following steps: obtaining a conjugate of formula 1:

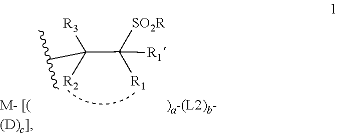

by conjugating a conjugate of formula 3:

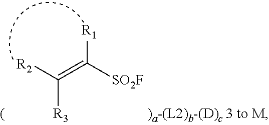

wherein M is a biological macromolecule, and M is linked to L1 with a nucleophilic functional group of M, L2 is a linker, and L2 is linked to R₁, R₃ or R₂ in the formula 1, D is a functional molecule, a is an integer of 1 to 10, b, c is each independently an integer of 0 to 10, provided that b and c are not simultaneously 0, wherein R₁ is H, an optionally substituted alkyl or an optionally substituted aryl, wherein R₁′ is H or the isotope thereof, wherein R₂ is H, an optionally substituted alkyl or an optionally substituted aryl, wherein R₃ is H, an optionally substituted alkyl or an optionally substituted aryl, optionally, a C linking R₁ and a C linking R₂ form a ring.

For example, the nucleophilic functional group of M may be selected from a group consisting of —SH, —NH₂, —SeH, —OH, and

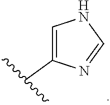 [SMILES: *c1c[nH]cn1]

In another aspect, the present application provides a method for preparing a conjugate, comprising the following steps: obtaining a conjugate of formula 2:

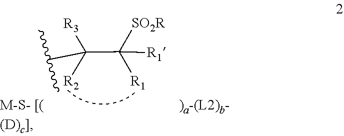

R is —OH or —F, by conjugating a conjugate of formula 3:

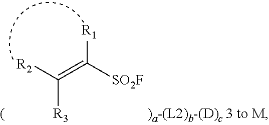

wherein M-S is a biological macromolecule with a cysteine, M-S is linked to L1 with the cysteine, L2 is a linker, and L2 is linked to R₁, R₃ or R₂ in the formula 3, D is a functional molecule, a is an integer of 1 to 10, b, c is each independently an integer of 0 to 10, provided that b and c are not simultaneously 0, wherein R₁ is H, an optionally substituted alkyl or an option-152,C1,M substituted aryl, wherein R₁′ is H or the isotope thereof, wherein R₂ is H, an optionally substituted alkyl or an optionally substituted aryl, wherein R₃ is H, an optionally substituted alkyl or an optionally substituted aryl, optionally, a C linking R¹ and a C linking R² form a ring.

In the present application, the method may be conducted at a temperature in a range of about 16° C. to about 37° C. For example, the method may be conducted at a temperature of at least about 16° C., at least about 17° C., at least about 18° C., at least about 19° C., at least about 20° C., at least about 21° C., at least about 22° C., at least about 23° C., at least about 24° C., at least about 25° C., at least about 26° C., at least about 27° C., at least about 33° C., at least about 34° C., at least about 35° C., at least about 36° C., or at least about 37° C.

In the present application, the method may be conducted at a pH in a range of about 7.4 to about 8. For example, the method may be conducted at a pH of at least about 7.4, at least about 7.5, at least about 7.6, at least about 7.7, at least about 7.8, at least about 7.9, or at least about 8.0.

In the present application, the method may be conducted with the catalyst. For example, the catalyst may comprise Pd(OAc) 2.

In the present application, the method may further comprise the following step: purifying said conjugate of formula 3.

In the present application, the conjugating in the method may comprise an addition reaction (for example, may be a nucleophilic addition reaction), which may belong to “click chemistry”. For example, a —SH of the M may be involved in the addition reaction in the method.

### Compound

In another aspect, the present application provides a compound of formular III, or a pharmaceutically acceptable salt thereof:

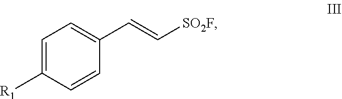

wherein R₁ is selected from a group consisting of: —OH, —PO₃H₂, —SeH, —SH, optionally substituted alkyl-OH, optionally substituted alkyl-halogen, optionally substituted alkyl-N₃, —B(OH)₂, -halogen, —OTf, optionally substituted alkyl-NH₂, —O-optionally substituted alkyl-C≡CH, —CO—NH—C≡CH-optionally substituted alkyl.

For example, R₁ may be selected from a group consisting of: —OH, —PO₃H₂, —SeH, —SH, —CH₂OH, —CH₂Br, —CH₂N₃, —B(OH)₂, —Br, —OTf, —CH₂NH₂, —Cl, —OCH₂C—CH or —CO—NH—C≡CH.

In some cases, the compound of Formula III, may be one of the compounds in the Table 1.

TABLE 1

| Structure | IUPAC NAME |
|---|---|
| 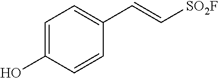 [SMILES: O=S(=O)(F)/C=C/c1ccc(O)cc1] | (E)-2-(4-hydroxyphenyl)ethene-1-sulfonyl fluoride |
| 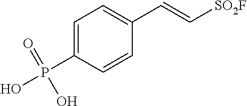 [SMILES: O=P(O)(O)c1ccc(/C=C/S(=O)(=O)F)cc1] | (E)-(4-(2- (fluorosulfonyl)vinyl)phenyl)phosphonic acid |
| 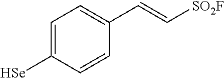 | (E)-2-(4-hydroselenophenyl)ethene-1- sulfonyl fluoride |
| 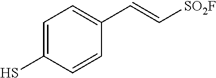 [SMILES: O=S(=O)(F)/C=C/c1ccc(S)cc1] | (E)-2-(4-mercaptophenyl)ethene-1-sulfonyl fluoride |
| 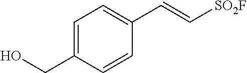 [SMILES: O=S(=O)(F)/C=C/c1ccc(CO)cc1] | (E)-2-(4-(hydroxymethyl)phenyl)ethene-1- sulfonyl fluoride |
| 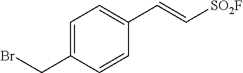 [SMILES: O=S(=O)(F)/C=C/c1ccc(CBr)cc1] | (E)-2-(4-(bromomethyl)phenyl)ethene-1- sulfonyl fluoride |
| 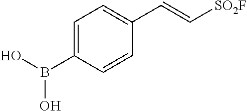 [SMILES: O=S(=O)(F)/C=C/c1ccc(B(O)O)cc1] | (E)-(4-(2- (fluorosulfonyl)vinyl)phenyl)boronic acid |
| 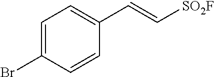 [SMILES: O=S(=O)(F)/C=C/c1ccc(Br)cc1] | (E)-2-(4-bromophenyl)ethene-1-sulfonyl fluoride |
| 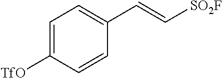 [SMILES: O=S(=O)(F)/C=C/c1ccc(OS(=O)(=O)C(F)(F)F)cc1] | (E)-4-(2-(fluorosulfonyl)vinyl)phenyl trifluoromethanesulfonate |
| 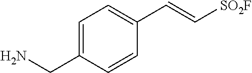 [SMILES: NCc1ccc(/C=C/S(=O)(=O)F)cc1] | (E)-2-(4-(aminomethyl)phenyl)ethene-1- sulfonyl fluoride |
| 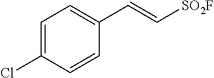 [SMILES: O=S(=O)(F)/C=C/c1ccc(Cl)cc1] | (E)-2-(4-chlorophenyl)ethene-1-sulfonyl fluoride |
| 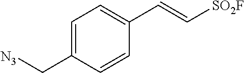 [SMILES: [N-]=[N+]=NCc1ccc(/C=C/S(=O)(=O)F)cc1] | (E)-2-(4-(azidomethyl)phenyl)ethene-1- sulfonyl fluoride |
| 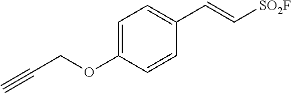 [SMILES: C#CCOc1ccc(/C=C/S(=O)(=O)F)cc1] | (E)-2-(4-(prop-2-yn-1-yloxy)phenyl)ethene-1- sulfonyl fluoride |
| 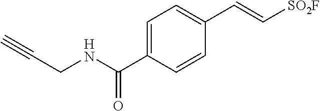 [SMILES: C#CCNC(=O)c1ccc(/C=C/S(=O)(=O)F)cc1] | (E)-2-(4-(prop-2-yn-1- ylcarbamoyl)phenyl)ethene-1-sulfonyl fluoride |

In another aspect, the present application provides a compound of Formular IV, or a pharmaceutically acceptable salt thereof:

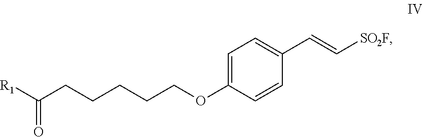

wherein R₁ is selected from a group consisting of: NH—(CH₂)ₙ₁—CO—R₂, —OH, NH—(CH₂)ₙ₂-R₃, NH—(CH₂CH₂—O)ₙ₃—(CH₂)ₙ₄—NH—CO—O—R₄, or

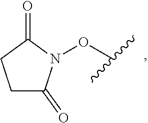

wherein n₁, n₂, n₃, or n₄ is independently an integer of 1 to 10, wherein R₂ is selected from a group consisting of:

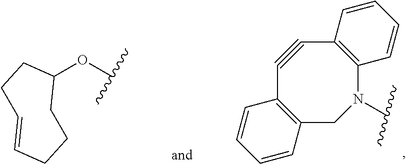

wherein R₃ is selected from a group consisting of:

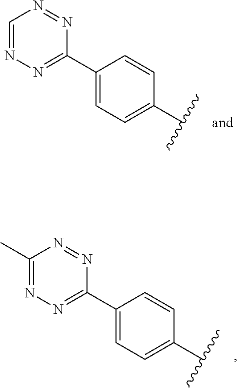 [SMILES: *c1ccc(-c2nncnn2)cc1 ; "and" ; *c1ccc(-c2nnc(C)nn2)cc1 ; ","]

wherein R₄ is

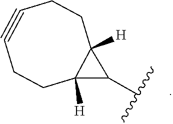 [SMILES: *C1[C@H]2CCC#CCC[C@@H]12 ; "."]

For example, R₁ may be selected from a group consisting of: NH—(CH₂)₂—CO—R₂, —OH, NH—(CH₂)—R₃, NH—(CH₂CH₂—O)3—(CH₂)₂—NH—CO—O—R₄.

In some cases, the compound of Formula IV, may be one of the compounds in the Table 2.

TABLE 2

| Structure | IUPAC NAME |
|---|---|
| 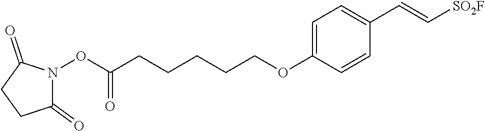 [SMILES: O=C(CCCCCOc1ccc(/C=C/S(=O)(=O)F)cc1)ON1C(=O)CCC1=O] | 2,5-dioxopyrrolidin-1-yl (E)-6-(4-(2- (fluorosulfonyl)vinyl)phenoxy)hexanoate |
| 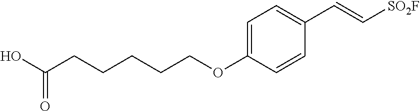 [SMILES: O=C(O)CCCCCOc1ccc(/C=C/S(=O)(=O)F)cc1] | (E)-6-(4-(2- (fluorosulfonyl)vinyl)phenoxy)hexanoic acid |
| 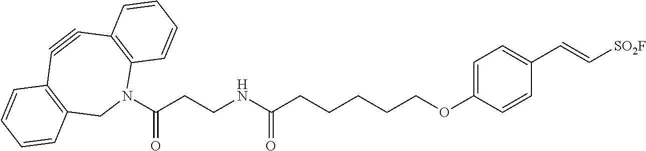 [SMILES: O=C(CCCCCOc1ccc(/C=C/S(=O)(=O)F)cc1)NCCC(=O)N1Cc2ccccc2C#Cc2ccccc21] | 1-(11,12-didehydrodibenz[b,f]azocin-5(6H)-yl)- (E)-2-(4-((6-(ethylamino)-6- oxohexyl)oxy)phenyl)ethene-1-sulfonyl fluoride |
| 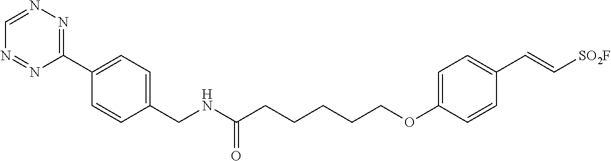 [SMILES: O=C(CCCCCOc1ccc(/C=C/S(=O)(=O)F)cc1)NCc1ccc(-c2nncnn2)cc1] | (E)-2-(4-((6-((4-(1,2,4,5-tetrazin-3- yl)benzyl)amino)-6-oxohexyl)oxy)phenyl)ethene- 1-sulfonyl fluoride |
| 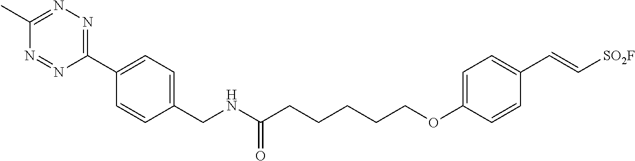 [SMILES: Cc1nnc(-c2ccc(CNC(=O)CCCCCOc3ccc(/C=C/S(=O)(=O)F)cc3)cc2)nn1] | (E)-2-(4-((6-((4-(6-methyl-1,2,4,5-tetrazin-3- yl)benzyl)amino)-6-oxohexyl)oxy)phenyl)ethene- 1-sulfonyl fluoride |
| 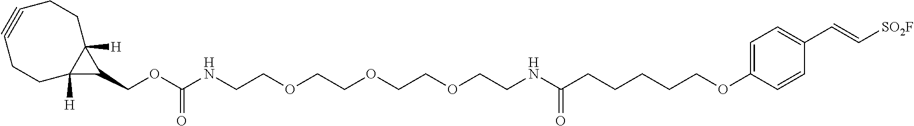 | ((1R,8S,9s)-bicyclo[6.1.0]non-4-yn-9-yl)methyl (18-(4-((E)-2-(fluorosulfonyl)vinyl)phenoxy)-13- oxo-3,6,9-trioxa-12-azaoctadecyl)carbamate |
| 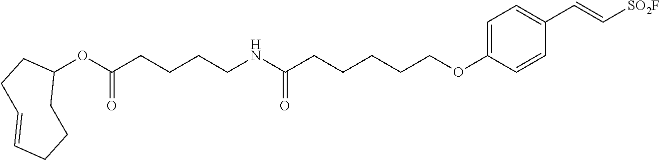 [SMILES: O=C(CCCCCOc1ccc(/C=C/S(=O)(=O)F)cc1)NCCCCC(=O)OC1CC/C=C/CCC1] | (E)-cyclooct-4-en-1-yl 5-(6-(4-((E)-2- (fluorosulfonyl)vinyl)phenoxy)hexanamido) pentanoate |

In another aspect, the present application provides a compound of Formular V, or a pharmaceutically acceptable salt thereof:

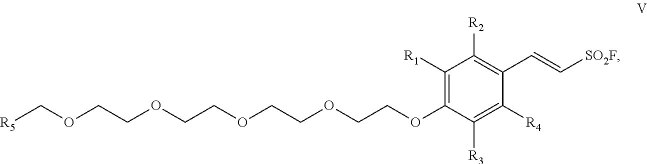

wherein R₁, R₂ and R₄ are any substituent, wherein R₃ is selected from a group consisting of: H, an optionally substituted alkyl-F₃, an optionally substituted alkyl-N or O—an optionally substituted alkyl, wherein R₅ is selected from a group consisting of: —COOH, —NH₂ and

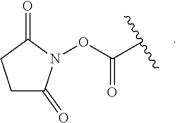 [SMILES: *C(=O)ON1C(=O)CCC1=O ; "."]

For example, R₁ may be H. For example, R₂ may be H. For example, R₄ may be H.

For example, R₃ may be selected from the group consisting of: H, CF₃, CN, and OCH₃.

In some cases, the compound of Formula V, may be one of the compounds in the Table 3.

TABLE 3

| Structure | IUPAC NAME |
|---|---|
| 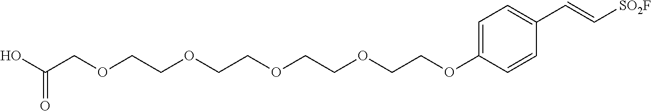 [SMILES: O=C(O)COCCOCCOCCOCCOc1ccc(/C=C/S(=O)(=O)F)cc1] | (E)-14-(4-(2- (fluorosulfonyl) vinyl)phenoxy)- 3,6,9,12-tetraoxatetra- decanoic acid |
| 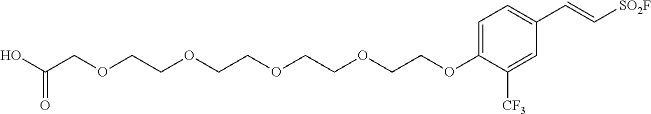 [SMILES: O=C(O)COCCOCCOCCOCCOc1ccc(/C=C/S(=O)(=O)F)cc1C(F)(F)F] | (E)-14-(4-(2- (fluorosulfonyl)vinyl)-2- (trifluoromethyl)phenoxy)- 3,6,9,12-tetraoxatetra- decanoic acid |
| 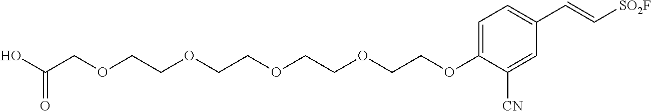 [SMILES: N#Cc1cc(/C=C/S(=O)(=O)F)ccc1OCCOCCOCCOCCOCC(=O)O] | (E)-14-(2-cyano-4-(2- (fluorosulfonyl) vinyl)phenoxy)- 3,6,9,12-tetraoxatetra- decanoic acid |
| 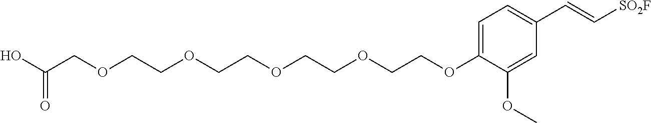 [SMILES: COc1cc(/C=C/S(=O)(=O)F)ccc1OCCOCCOCCOCCOCC(=O)O] | E)-14-(4-(2- (fluorosulfonyl)vinyl)- 2-methoxyphenoxy)-3,6,9,12- tetraoxatetradecanoic acid |
| 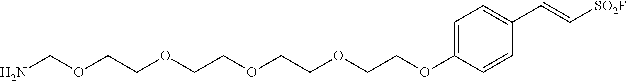 [SMILES: NCOCCOCCOCCOCCOc1ccc(/C=C/S(=O)(=O)F)cc1] | (E)-2-(4-((1-amino-2,5,8,11- tetraoxatridecan-13- yl)oxy)phenyl) ethene-1-sulfonyl fluoride |
| 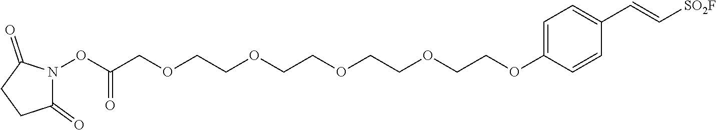 [SMILES: O=C(COCCOCCOCCOCCOc1ccc(/C=C/S(=O)(=O)F)cc1)ON1C(=O)CCC1=O] | 2,5-dioxopyrrolidin- 1-yl (E)-14-(4- (2-(fluorosulfonyl) vinyl)phenoxy)- 3,6,9,12- tetraoxatetradecanoate |

In another aspect, the present application provides a compound of Formular VI, or a pharmaceutically acceptable salt thereof:

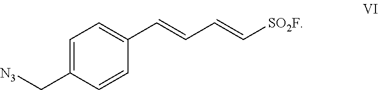 [SMILES: [N-]=[N+]=NCc1ccc(/C=C/C=C/S(=O)(=O)F)cc1]

As used herein, the term “Formula III” (or Formula IV, Formula V, Formula VI) may be are also defined to include all forms of the compound of “Formula III” (or Formula IV, Formula V, Formula VI), including hydrates, solvates, isomers, crystalline and non-crystalline forms, isomorphs, polymorphs, and metabolites thereof. For example, the compounds of Formula VI, or pharmaceutically acceptable salts thereof, may exist in unsolvated and solvated forms. When the solvent or water is tightly bound, the complex will have a well-defined stoichiometry independent of humidity. When, however, the solvent or water is weakly bound, as in channel solvates and hygroscopic compounds, the water/solvent content will be dependent on humidity and drying conditions. In such cases, non-stoichiometry will be the norm.

The compounds of “Formula III” (or Formula IV, Formula V, Formula VI) may have asymmetric carbon atoms. For example, the carbon-carbon bonds of the compounds of Formula VI may be depicted herein using a solid line, a solid wedge, or a dotted wedge. The use of a solid line to depict bonds to asymmetric carbon atoms is meant to indicate that all possible stereoisomers (e.g. specific enantiomers, racemic mixtures, etc.) at that carbon atom are included. The use of either a solid or dotted wedge to depict bonds to asymmetric carbon atoms is meant to indicate that only the stereoisomer shown is meant to be included. It is possible that compounds of the present application may contain more than one asymmetric carbon atom. In those compounds, the use of a solid line to depict bonds to asymmetric carbon atoms is meant to indicate that all possible stereoisomers are meant to be included. For example, unless stated otherwise, it is intended that the compounds of “Formula III” (or Formula IV, Formula V, Formula VI) can exist as enantiomers and diastereomers or as racemates and mixtures thereof. The use of a solid line to depict bonds to one or more asymmetric carbon atoms in a compound of “Formula III” (or Formula IV, Formula V, Formula VI) and the use of a solid or dotted wedge to depict bonds to other asymmetric carbon atoms in the same compound is meant to indicate that a mixture of diastereomers is present.

The compounds of the present application (e.g., the compounds of “Formula III” (or Formula IV, Formula V, Formula VI) may exist as clathrates or other complexes. Included within the scope of the invention are complexes such as clathrates, drug-host inclusion complexes wherein, in contrast to the aforementioned solvates, the drug and host are present in stoichiometric or non-stoichiometric amounts. Also included are complexes of “Formula III” (or Formula IV, Formula V, Formula VI) containing two or more organic and/or inorganic components which may be in stoichiometric or non-stoichiometric amounts. The resulting complexes may be ionized, partially ionized, or non-ionized. For a review of such complexes, see J. Pharm. Sci., 64 (8), 1269-1288 by Haleblian (August 1975).

Stereoisomers of “Formula III” (or Formula IV, Formula V, Formula VI)include cis and trans isomers, optical isomers such as R and S enantiomers, diastereomers, geometric isomers, rotational isomers, conformational isomers, and tautomers of the compounds of “Formula III” (or Formula IV, Formula V, Formula VI), including compounds exhibiting more than one type of isomerism; and mixtures thereof (such as racemates and diastereomeric pairs). Also included are acid addition or base addition salts wherein the counterion is optically active, for example, D-lactate or L-lysine, or racemic, for example, DL-tartrate or DL-arginine.

### Pharmaceutical Composition and Use

In another aspect, the present application provides a pharmaceutical composition comprising the conjugate of the present application and a pharmaceutically acceptable carrier.

In present application, the pharmaceutical compositions may comprise the conjugate of the present application presented with a pharmaceutically acceptable carrier. The carrier may be a solid product, a liquid, or both, and may be formulated with the compound as a unit-dose composition, for example, a tablet, which can contain from 0.05% to 95% by weight of the active compounds. Other pharmacologically active substances may also be present.

In the present application, the conjugate and/or the pharmaceutical composition of the present invention may be administered by any suitable route.

In another aspect, the present application provides a method for adjusting a tumor micro-environment of a subject, comprising administering to the subject the conjugate of the present application, or the pharmaceutical composition of the present application.

In another aspect, the present application provides a method for adjusting the immune reaction of a subject, comprising administering to the subject the conjugate of the present application, or the pharmaceutical composition of the present application.

In another aspect, the present application provides a method for preventing and/or treating disease in a subject in need of, comprising administering to the subject the conjugate of the present application, or the pharmaceutical composition of the present application.

The method may be an in vitro method, an ex vivo method, or an in vivo method. For example, the conjugate of the present application may be administered in vitro to one or more cells. As another example, the conjugate of the present application may be administered to a subject in need thereof.

In another aspect, the present application provides a conjugate of present application, in use of preventing and/or treating disease in a subject in need of.

In another aspect, the present application provides a method of preparing a medicament for treating disease.

For example, the disease may be a tumor. For example, the tumor may be a solid tumor. For example, the tumor may be a non-solid tumor. For example, the solid tumor may comprise sarcomas and carcinomas. Sarcomas may refer to tumors in a blood vessel, bone, fat tissue, ligament, lymph vessel, muscle or tendon. Carcinomas may refer to tumors that form in epithelial cells. It is contemplated that the solid tumor is a non-lymphoma solid tumor. For example, the solid tumor may be named for the type of cells that form them.

For example, the disease may comprise a tumor and/or an autoimmune disease.

For example, the autoimmune disease may comprise glomerulonephritis, Goodpasture's syndrome, necrotizing vasculitis, lymphadenitis, peri-arteritis nodosa, systemic lupus erythematosis, rheumatoid, arthritis, psoriatic arthritis, systemic lupus erythematosis, psoriasis, ulcerative colitis, systemic sclerosis, dermatomyositis/polymyositis, anti-phospholipid antibody syndrome, scleroderma, pemphigus vulgaris, ANCA-associated vasculitis (e.g., Wegener's granulomatosis, microscopic polyangiitis), uveitis, Sjogren's syndrome, Crohn's disease, Reiter's syndrome, ankylosing spondylitis, Lyme arthritis, Guillain-Barre syndrome, Hashimoto's thyroiditis, and cardiomyopathy.

Typically, a conjugate of the present application may be administered in an amount effective to treat a disease as described herein. The conjugate of present application may be administered by any suitable route in the form of a pharmaceutical composition adapted to such a route, and in a dose effective for the treatment intended. Therapeutically effective doses of the conjugate required to treat the progress of the medical condition are readily ascertained by one of ordinary skill in the art using preclinical and clinical approaches familiar to the medicinal arts. The term “therapeutically effective amount” as used herein generally refers to that amount of the conjugate being administered which will relieve to some extent one or more of the symptoms of the disease being treated.

The dosage regimen for the conjugate and/or compositions comprising the conjugate may be based on a variety of factors, including the type, age, weight, sex and medical condition of the patient; the severity of the condition; the route of administration; and the activity of the particular compound employed. Thus, the dosage regimen may vary widely.

Suitable subjects according to the present invention include mammalian subjects. For example, the subject may be a mammal, for example, the subject may be a human.

In another aspect, the present application provides a diagnostic reagent comprising the conjugate of the present application.

For example, the diagnostic reagent may be labeled.

For example, the label may be selected from the group consisting of a radiolabel, a fluorophore, a chromophore, an imaging agent, and a metal ion.

### EXAMPLES

The following examples are set forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric. Standard abbreviations may be used, e.g., bp, base pair(s); kb, kilobase(s); pl, picoliter(s); s or see, second(s); min, minute(s); h or hr, hour(s); aa, amino acid(s); nt, nucleotide(s); i.m., intramuscular(ly); i.p., intraperitoneal(ly); s.c., subcutaneous(ly); and the like.

### Silica Gel Column Chromatography

Silica gel column chromatography was carried out using silica Gel 60 (200-300 mesh). Analytical thin layer chromatography (TLC) was performed using silica gel (silica gel 60 F254). TLC was performed on precoated silica gel plates using shortwave UV light as the visualizing agent and KMnO₄ and heat as developing agents.

### Synthesis of SSF-DNA

To an Eppendorf tube with 5′—NH₂-20/59nt ssDNA (50 μM final concentration, 1 equiv.) in PBS (50 mM, pH 8.0), SSF-NHS (100 eq, 100 mM in DMF) and DMF were added. The reaction mixture was vortexed and shaken at 30° C. After overnight incubation, the reaction mixture was analyzed by LC-MS.

### Synthesis of Mal-DNA

To an Eppendorf tube with 5′—NH₂-20 (50 μM final concentration, 1 equiv.) in PBS (50 mM, pH 8.0), SSF-NHS (100 eq, 100 mM in DMF) and DMF were added. The reaction mixture was vortexed and shaken at 30° C. After overnight incubation, the reaction mixture was analyzed by LC-MS.

### Hydrolytic Stability of SSF-DNA Vs Mal-DNA

SSF-20nt ssDNA/Mal-20nt ssDNA (100 μM final concentration) in PBS (50 mM, pH 7.4) was shaken for 48 h at 37° C. for 48 h. 20 L of the reaction mixture was analyzed by LC-MS.

### Synthesis of Protein-DNA Conjugates

SSF/Mal-DNA (56 μL, 450 mM in H₂O) and PBS (10 μL, pH=8.0, 50 mM) were added to a solution of Nb-PD-L1 or other proteins (50 μL, 100 μM, HEPES buffer). The reaction was allowed to be incubated at 37° C. for 12 h, which generated homogenous protein-DNA conjugates.

### LC-MS

LC-MS analysis of protein conjugation and protein-DNA (GFP-20ntssDNA, neo2-20ntssDNA): LC-MS was performed on a Xevo G2-S TOF mass spectrometer coupled to an Acquity high-performance liquid chromatography (UPLC) system using an Acquity UPLC Protein BEH C4 column (1.7 mm, 2.1×50 mm). Solvents A, water with 0.1% formic acid and B acetonitrile with 0.1% formic acid were used as the mobile phase at a flow rate of 0.5 ml/min. Gradient used: isocratic 95% H₂O for 2 min, then 95% to 10% H₂O in 4 min, then 10% H₂O for 1 min, then 10% to 95% H₂O in 1 min, then 95% H₂O for 2 min. The electrospray source was operated in the positive mode with a capillary with a capillary voltage of 2.0 kV and a cone voltage of 40 V. Nitrogen was used as the desolvation gas at a total flow of 850 L/h. Total mass spectra were reconstructed from the ion series using the MaxEnt algorithm preinstalled on MassLynx software (v. 4.1 from Waters) according to the manufacturer's instructions. To obtain the ion series described, the major peak(s) of the chromatogram were selected for integration and further analysis.

LC-MS analysis of SSF-DNA, Mal-DNA and Nb-PD-L1-ssDNA: LC-MS was performed on a Xevo G2-S TOF mass spectrometer coupled to an Acquity high-performance liquid chromatography (UPLC) system using an Acquity UPLC Protein BEH C 8 column (1.7 mm, 2.1×50 mm). Solvents A, 10 mM ammonium formate in water and B 100% methanol were used as the mobile phase at a flow rate of 0.5 ml/min. Gradient used: isocratic 95% H₂O for 2 min, then 95% to 5 H₂O in 4 min, then 5% H₂O for 1 min, then 5% to 95% H₂O in 1 min, then 95% H₂O for 2.5 min. The electrospray source was operated in the negative mode with a capillary voltage of 2.0 kV and a cone voltage of 80 V. Nitrogen was used as the desolvation gas at a total flow of 850 L/h. Total mass spectra were reconstructed from the ion series using the MaxEnt algorithm preinstalled on MassLynx software (v. 4.1 from Waters) according to the manufacturer's instructions. To obtain the ion series described, the major peak(s) of the chromatogram were selected for integration and further analysis.

LC-MS analysis of peptide-MA conjugation: LC-MS was performed on a Xevo SQ Detector 2 mass spectrometer coupled to an Acquity high-performance liquid chromatography (UPLC) system using an Acquity UPLC BEH300 C18 column (1.7 mm, 2.1×50 mm). Solvents A, water with 0.1% formic acid and B acetonitrile were used as the mobile phase at a flow rate of 0.4 ml/min. Method A: Gradient used: isocratic 90% H₂O for 2 min, then 90% to 10% H₂O in 5 min, then 10% H₂O for 1 min, then 10% to 90% H₂O in 1 min, then 95% H₂O for 1 min. Method B: Gradient used: isocratic 90% H₂O for 2 min, then 90% to 70% H₂O in 15 min, then 70% to 10% H₂O for 20 min, then 10% to 90% H₂O in 1 min, then 95% H₂O for 2 min. The electrospray source was operated in the positive mode with a capillary voltage of 2.0 kV and a cone voltage of 40 V. Nitrogen was used as the desolvation gas at a total flow of 850 L/h. Total mass spectra were reconstructed from the ion series using the MaxEnt algorithm preinstalled on MassLynx software (v. 4.1 from Waters) according to the manufacturer's instructions. To obtain the ion series described, the major peak(s) of the chromatogram were selected for integration and further analysis.

LC-MS/MS analysis of protein conjugation: For in-gel digestion, the labeled GFPs were resolved by SDS-PAGE first, and the gel was stained by Coomassie brilliant blue. The GFP band was excised, cut into small particles and transferred into a precleaned microcentrifuge tube. The resulting gel particles were desalted twice with 50% ACN in 25 mM ammonium bicarbonate (ABC) and then dehydrated in ACN. The gel particles were rehydrated with 20 mM DTT in 25 mM ABC and incubated for 45 min at 55° C. Gel particles were washed with 25 mM ABC and dehydrated again with ACN, followed by incubation with 55 mM iodoacetamide in 25 mM ABC for 30 min at room temperature in the dark. The treated gel particles were washed with 25 mM ABC and dehydrated in ACN again. Then, the gel particles were rehydrated in a trypsin solution (20 ng/μL) and incubated at 37° C. for 16 h. To extract the tryptic peptides, the gel particles were soaked in ACN/water/FA solution (v: v: v=50:45:5) with vortexing for 30 min. The solution was carefully removed, and the extraction was repeated once. The extracts were combined and dried in a vacuum centrifuge. LC-MS/MS was performed on an Orbitrap Fusion Lumosmass spectrometer (Thermo Fisher Scientific) coupled with an Easy-nLC 1200 LC system. The peptide samples were loaded onto an analytical column (1.9 μm, 120 A, C18, 250 mm*75 μm i.d.) and eluted with 65 min gradient. The mass spectrometer was performed in data-dependent mode. Full scan spectra were acquired over the m/z range from 350-1500 using the Orbitrap mass analyzer. MS/MS fragmentation is performed with HCD mode. The normalized collision energy was 30 V. The raw data were analyzed by Pfind3 and searched against the bovine proteome in the UniProt database. Carbamidomethylation of cysteine was set as a fixed modification. Oxidation of methionine and modification of cysteine residues were set as variable modifications. 2b- or 2b+la-modified peptides were considered to be correctly identified when a score (PSM score, Peptide Spectrum Match score) higher than 26 and the modified sites were manually validated.

### NMR

NMR experiments were measured on a Bruker AVANCE III-400 or 500 spectrometers, and in deuterochloroform (CDCl₃). ¹H NMR and ¹³C NMR spectra were recorded at 400 MHz or 500 MHz and 100 MHz or 125 MHz spectrometers, respectively. ¹⁹F NMR spectra were recorded at 376 MHz or 470 MHz spectrometers. Chemical shifts are reported as S values relative to internal TMS (6 0.00 for 1H NMR), chloroform (6 7.26 for ¹H NMR), and chloroform (6 77.00 for ¹³C NMR). The following abbreviations are used for the multiplicities: s: singlet, d: doublet, dd: doublet of doublet, t: triplet, q: quadruplet, m: multiplet, br: broad signal for proton spectra. Mass spectra were measured by ESI-MS (LCQ Fleet, Thermo Fisher Scientific).

Cell Barcoding and scRNA-Seq

A cell mixture of Jurkat, A549, JIMT-1 and MDA-MB-231 was stained 30 min at 4° C. with Nb-PD-L1-59ntssDNA. After washing and detecting the cell number and cell viability, cells were pooled and loaded to a microwell chip targeting 20,000 cells on Singleron Matrix® (GEXSCOPE Single Cell RNA-seq Kit, Singleron Biotechnologies, Nanjing, China). The scRNA-seq libraries were preparation according to the manufacturer's instructions (Singleron Biotechnologies, Nanjing, China). After amplification, cDNA and Nb-PD-L1-59ntssDNA Tags were separated by SPRI size-selection with 0.6× and 1.4× SPRI, respectively. Nb-PD-L1-59ntssDNA Tag libraries were quantified (Qubit, Invitrogen) and amplified using primer SGR-beads-1/SGR-tag-1 and indexed by additional PCR with primer SGR-beads-2/SGR-tag-2. Final Nb-PD-L1-59ntssDNA Tag libraries and transcriptome libraries were analyzed on a BioAnalyzer high-sensitivity DNA kit (Agilent) and sequenced on Illumina NovaSeq 6000.

Primers for Nb-PD-L1-Tag
library preparation

| | | SEQ |
|---|---|---|
| | | ID |
| Primer | Sequence | NO. |
| SGR-beads-1 | AATGATACGGCGACCACCGAGATC | 1 |
| sequence | TACACTCTTTCCCTACACGACGCT | |
| | C | |
| SGR-tag-1 | TGGAGTTCAGACGTGTGCTCTTCC | 2 |
| sequence | GATCTGTTGTCAAGATGCTACCGT | |
| | TCAGAG | |
| SGR-beads-2 | AATGATACGGCGACCACCGAGATC | 3 |
| sequence | T | |
| SGR-tag-2 | CAAGCAGAAGACGGCATACGAGAT | 4 |
| sequence | TCGCCTTAGTGACTGGAGTTCAGA | |
| | CGTGTGCTC | |

Protein sequences
Sequence of Engineered neo2: Engineered Neo2
is composed of 124 amino acids and 1 free
cysteine.
MLVNRICGKGIDGGSPKKKIQLHAEHALYDALMIL
NIVKTNSPPAEEKLEDYAFNFELILEEIARLFESG
DQKDEAEKAKRMKEWMKRIKTTASEDEQEEMANAI
ITILQSWIFSAVDHHHHHH
(SEQ ID NO. 5)
Sequence of Engineered Nb-Pd-L1:
Engineered Nb-Pd-L1 is composed of
143 amino acids and 1 free cysteine.
MDQVQLQESGGGLVQPGGSLRLSCAASGKMSSRRC
MAWFRQAPGKERERVAKLLTTSGSTYLADSVKGRF
TISQNNAKSTVYLQMNSLKPEDTAMYYCAADSFED
PTCTLVTSSGAFQYWGQGTQVTVSSLPETGGCHHH
HHH
(SEQ ID NO. 6)
Sequence of GFP: Engineered GFP is composed
of 243 amino acids and 3 free cysteines
MSKGEELFTGVVPILVELDGDVNGHKFSVRGEGEG
DATNGKLTLKFICTTGKLPVPWPTLVTTLTYGVQC
FSRYPDHMKRHDFFKSAMPEGYVQERTISFKDDGT
YKTRAEVKFEGDTLVNRICLKGIDFKEDGNILGHK
LEYNFNSHNVYITADKQKNGIKANFKIRHNVEDGS
VQLADHYQQNTPIGDGPVLLPDNHYLSTQSVLSKD
PNEKRDHMVLLEFVTAAGITHGGGGLEHHHHHH
(SEQ ID NO. 7)
Trastuzumab light chain:
DIQMTQSPSSLSASVGDRVTITCRASQDVNTAVAW
YQQKPGKAPKLLIYSASFLYSGVPSRFSGSRSGTD
FTLTISSLQPEDFATYYCQQHYTTPPTFGQGTKVE
IKRTVAAPSVFIFPPSDEQLKSGTASVVCLLNNFY
PREAKVQWKVDNALQSGNSQESVTEQDSKDSTYSL
SSTLTLSKADYEKHKVYACEVTHQGLSSPVTKSFN
RGEC
(SEQ ID NO. 8);
Trastuzumab heavy chain:
EVQLVESGGGLVQPGGSLRLSCAASGFNIKDTYIH
WVRQAPGKGLEWVARIYPTNGYTRYADSVKGRFTI
SADTSKNTAYLQMNSLRAEDTAVYYCSRWGGDGFY
AMDYWGQGTLVTVSSASTKGPSVFPLAPSSKSTSG
GTAALGCLVKDYFPEPVTVSWNSGALTSGVHTFPA
VLQSSGLYSLSSVVTVPSSSLGTQTYICNVNHKPS
NTKVDKKVEPKSCDKTHTCPPCPAPELLGGPSVFL
FPPKDTLMISRTPEVTCVVVDVSHEDPEVKFNWYV
DGVEVHNAKTKPREEQYNSTYRVVSVLTVLHQDWL
NGKEYKCKVSNKALPAPIEKTISKAKGQPREPQVY
TLPPSREEMTKNQVSLTCLVKGFYPSDIAVEWESL
TQPEGKNNTTKPPKVSNKALPAPIEKTISKSRKQP
REPQVYTLPPSREEMTKNQVSLTCLVKSLKSPK
(SEQ ID NO. 9)
The amino acid sequences of the light chain
and the heavy chain of KN026 can be referred
to US 2018/0291103, and that of KN046 can be
referred to US20210095031A1.
DNA sequence
20ntssDNA:
5′-NH2-C6-AGC AGC ACA GAG GTC AGA TG
(SEQ ID NO. 10)
59ntssDNA:
5′-NH2-C6-TGT CAA GAT GCT ACC GTT CAG
AGC GCA AGA CÁC TCC ACA AAA AAA AAA AAA
AAA AAA *A *A *thio modification
(SEQ ID NO. 11)

### Data Analysis

Raw sequenced reads were processed using CeleScope pipeline (v1.3.1) with default parameter (https://github.com/singleron-RD/CeleScope). Nb-PD-LI-59ntssDNA Tag libraries were processed with a new feature barcode processing plug-in (“teg”) of CeleScope inspired by previous scRNA-seq multiplexing algorithm. Gene expression matrices were then analyzed using R language.

Modification of GFP with MAs

MAs (1 μL, 20 mM in DMSO) and PBS (15 μL, pH=7.4/9.0, 50 mM) were added to a solution of GFP (4 μL, 250 μM, HEPES buffer). After incubation at 37° C. for 2 h, the solution was desalted to give GFP-MAs.

### Linker Stability Studies

GSH (0.8 μL, 100 mM) and PBS (20 μL, pH=8.0, 50 mM) were added to a solution of GFP-Linker (20 μL, 40 μM, in PBS) at 37° C. for 48 h. (FIG. 20)

### Example 1 Screening a Michael Acceptor as a Linker

Michael acceptors are common reagents for an addition reaction with a cysteine on an antibody. A new Michael acceptor was been investigated so that they may react efficiently and chemo-selectively with cysteine on antibodies. And no degradation occurred in the serum after the modification of the Michael acceptor with the antibody. Based on this, a candidate Michael acceptor was used as a reaction reagent with a green fluorescent protein which is regarded as a template protein to screen the Michael acceptor. Taking the reactivity, chemo-selectivity, and stability into consideration, it was screened out the Michael acceptor which was capable of reacting with a cysteine efficiently.

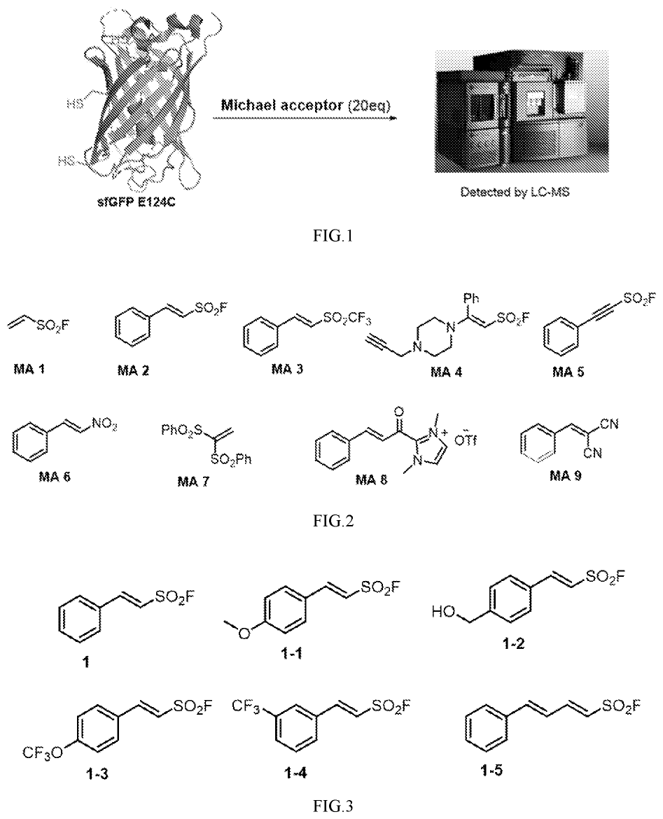

The reaction process is shown in FIG. 1. And the chemical structure of the following Michael acceptor candidates is shown in FIG. 2.

TABLE 4
Screening of Michael acceptor

| | Michael | Time | Addition | Conversion |
|---|---|---|---|---|
| Entry¹ | acceptor | (h) | number | (%) |
| 1 | MA 1 | 2 | —² | >99 |
| 2 | MA 2 | 2 | 2 | >99 |
| 3 | MA 3 | 2 | 1 | 60 |
| 4 | MA 4 | 2 | 0 | 0 |
| 5 | MA 5 | 2 | 0 | 0 |
| 6 | MA 6 | 2 | 2 | 60 |
| 7 | MA 7 | 2 | 2 | <20 |
| 8 | MA 8 | 2 | 0 | 60 |
| 9 | MA 9 | 2 | 0 | 0 |

¹Reaction condition: sfGFP E124C (320 μM, 5 μL), Michael acceptor (6.4 μL, 5 mM), PBS buffer (40 μL, pH = 8), 37° C., 2 h.
²addition number no attribution.

And Michael acceptor MA 2 was named as the Reagent 1 or PhESF.

### Example 2 Modification of a Linker

The Reagent 1 in example 1 was modified (the chemical structure of the modified Michael acceptor candidates is shown in FIG. 3).

When the benzene ring of the Reagent 1 was modified with different substituents (Michael acceptor 1-1 to 1-4), the difference of different substituents on the addition reaction (for example, the addition activity) was investigated (Table 5). Michael acceptor 1-1 to 1-4 were reacted with sfGFP-TEV. When a para-position of a sulfur-fluorine group was connected with a strong electron-withdrawing group trifluoromethoxy, the addition activity was significantly reduced (entry 4); When the electron-donating group methoxy and hydroxymethyl were attached to the para position of the thiofluoro group, and the electron withdrawing group trifluoromethyl was attached to the meta position, the addition reaction activity did not change significantly (entry 2-3, entry 5). When the sulfonyl fluoride group was attached to the allene 1-5, its addition reaction activity was significantly reduced (entry 6).

The reaction process may be shown in FIG. 4.

TABLE 5
Modification of sfGFP-TEV by derivatives of the reagent 1

| | Michael | | Conv. | Addition |
|---|---|---|---|---|
| Entry | acceptor | Time (h) | (%) | number |
| 1 | 1 | 2 | >99 | 2 |
| 2 | 1-1 | 2 | >99 | 1.8 |
| 3 | 1-2 | 2 | >99 | 2 |
| 4 | 1-3 | 2 | 80 | 0.9 |
| 5 | 1-4 | 2 | >99 | 2.1 |
| 6 | 1-5 | 2 | >99 | 1.2 |

### Example 3 Modification of a Linker

Different configurations of an alkenyl sulfonyl fluoride (the chemical structure of the modified Michael acceptor candidates is shown in FIG. 5) were used to investigate the effect of different configurations of olefin sulfonyl fluoride on the addition reaction (Table 6). When 5 equivalents of the Michael acceptor were used to react with sfGFP E124C, Michael acceptor 1, la, 1b, and 1a-1 all exhibit good chemoselectivity; while Michael acceptor 1b-1 not only reacted with cysteine, but also with lysine. When 20 equivalents of the Michael acceptor were used to react with sfGFP E124C, only Michael acceptor 1 has good chemical selectivity.

The reaction process may be shown in FIG. 6.

TABLE 6

| | Michael | Time | | Conv. | Modification |
|---|---|---|---|---|---|
| Entry | acceptor | (h) | Equiv. | (%) | number |
| 1 | 1 | 2 | 5 | >99% | 1 |
| 2 | 1 | 2 | 20 | >99% | 1 |
| 3 | 1a | 2 | 5 | >99% | 1 |
| 4 | 1a | 2 | 20 | >99% | 5 |
| 5 | 1a-1 | 2 | 5 | >99% | 2 |
| 6 | 1a-1 | 2 | 20 | >99% | 3 |
| 7 | 1b | 2 | 5 | >99% | 1 |
| 8 | 1b | 2 | 20 | >99% | 11 |
| 9 | 1b-1 | 2 | 5 | >99% | 6 |
| 10 | 1b-1 | 2 | 20 | >99% | 9 |

### Example 4 the Features of the Selected Linker

### 4.1 Selectivity

In order to further verify the reactivity, chemoselectivity, and stability of the Michael acceptor 1, the linkers (e.g. the linker used in an ADC) reported in the previous literature were chosen as a comparative example to prove that the Michael acceptor 1 has a good reaction selectivity.

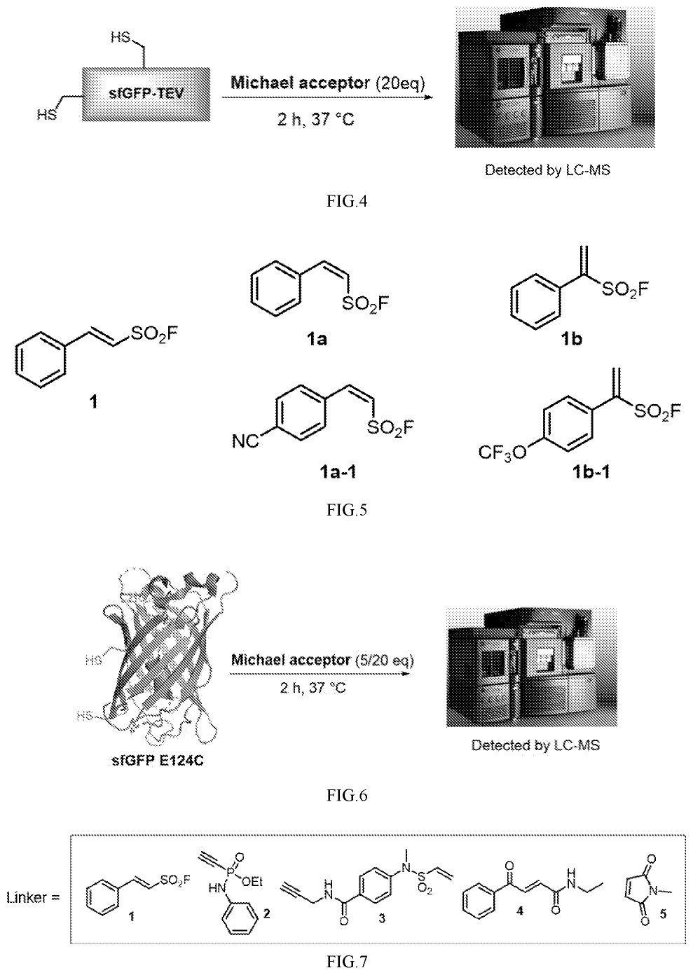

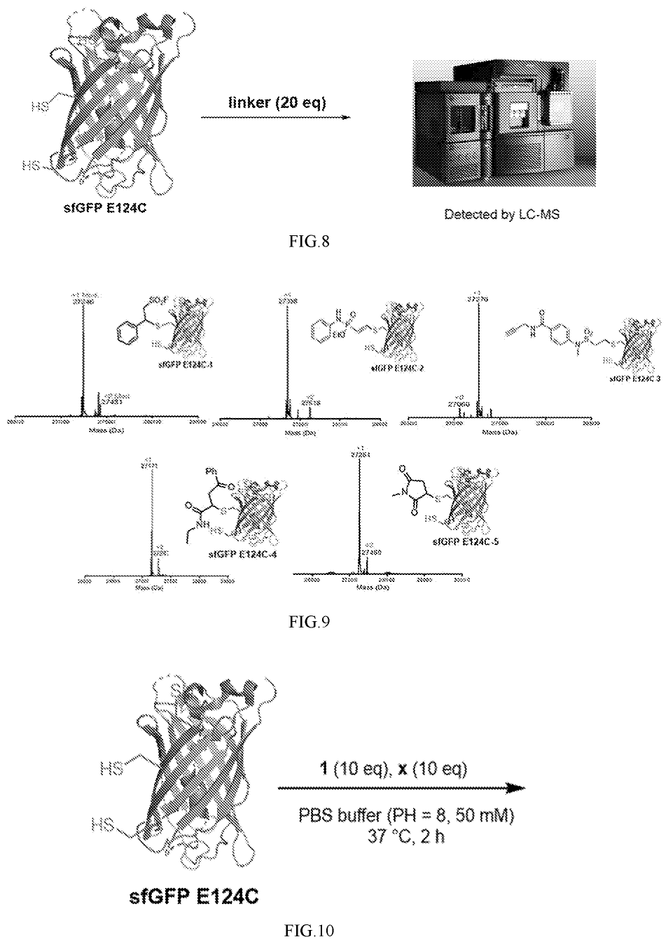

The chemical structure of the previous reported linker is shown in FIG. 7. The reaction process of the reaction between the sfGFP E124C and the previous reported linker may be shown in FIG. 8. The reaction results were shown in FIG. 9; Reaction condition: sfGFP E124C (320 μM, 5 μL), linker (5 mM, 6.4 μL), PBS buffer (40 μL, pH=8), 37° C., 2 h.

### 4.2 Competitivity

In order to verify their difference in reactivity, comparative example in competitivity test had been done (FIG. 10), and the results showed that the reactivity of the Michael acceptor 1 was moderate: which means that its reactivity was higher than alkenyl sulfonic acid amide and alkynyl phosphate, lower than maleimide (5) and carbonyl acrylic amide (4) (Table 7).

TABLE 7

| Entry¹ | Reagents | Ratio(sfGFP E124C-1:sfGFP E124C-x) |
|---|---|---|
| 1 | 1.2 | sfGFP E124C-1:sfGFP E124C-2 > 99:1 |
| 2 | 1.3 | sfGFP E124C-1:sfGFP E124C-3 = 6:1 |
| 3 | 1.4 | sfGFP E124C-1:sfGFP E124C-4 = 1:5 |
| 4 | 1.5 | sfGFP E124C-1:sfGFP E124C-5 < 1:99 |

¹Reaction condition: sfGFP E124C (320 μM, 5 μL), 1 (5 mM, 3.2 μL,), × (5 mM, 3.2 μL,), PBS buffer (40 μL, pH = 8), 37° C., 2 h.

### 4.3 Stability

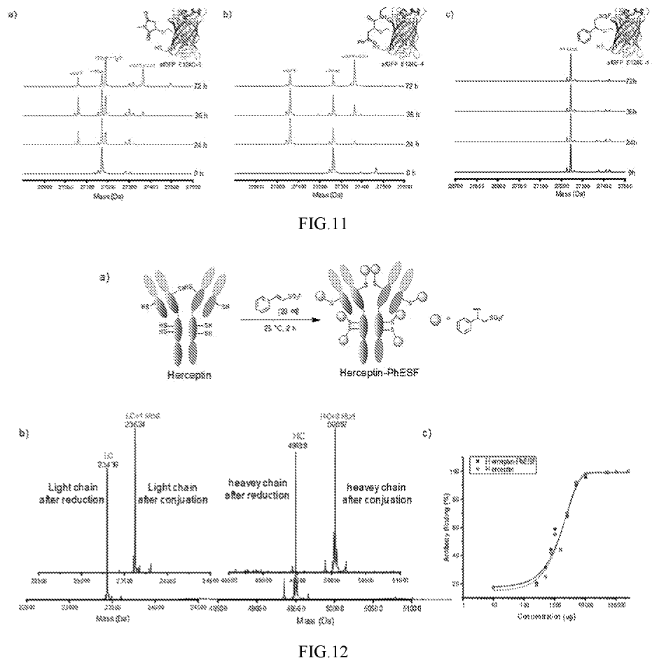

The stability of a sfGFP modified PhESF as a linker was verified, using the linker 5 and 4 with higher reactivity as controls. After sfGFP had been modified, it was added to the buffer comprising GSH, and was placed at 37° C. for different time, and a mass spectrometry was performed. From the mass spectrometry results, it can be seen that both the linker 4 and 5 with modified sfGFP obviously undergo thiol exchange. However, under the same condition the Michael acceptor 1 did not have a thiol exchange (FIGS. 11a-11c).

### Example 5 Producing a Conjugate Comprising a Linker and an Antibody

The Michael acceptor 1 was used to modify the antibody (Herceptin for example) (FIGS. 12a-12b), and it was verified that the modification of the antibody would not affect its ability to bind to the antigen (FIG. 12c).

### Example 6 Further Modification of a Linker

The Michael acceptor 1 has no further modifiable groups. Hence, the Michael acceptor 1 was modified to have an azide group, then the modified Michael acceptor 1 was used to modify the antibody and the stability of the conjugated antibody was tested in serum.

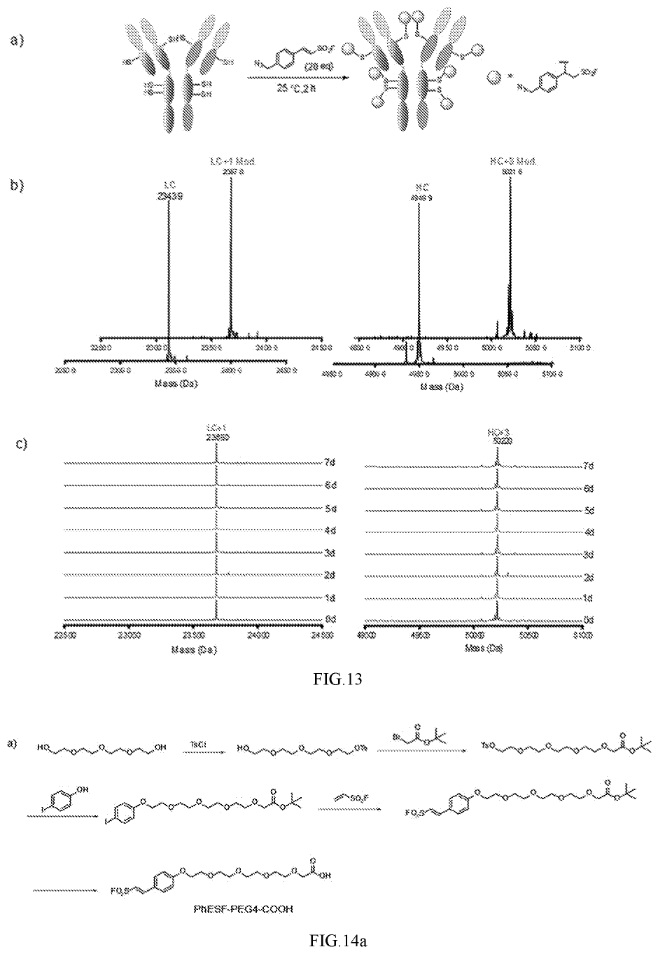

Mass spectrometry detection revealed that the modified antibody did not undergo elimination reaction after 7 days in the serum (FIGS. 13a-13C). In FIG. 13, FIG. 13a shows a N₃-PhESF chemoselectively modified Herceptin; FIG. 13b shows the result of a N3-PhESF modified antibody mass spectrum; FIG. 13c shows the result of a Herceptin-PhESF-N3 in serum stability test.

### Example 7 Producing a Conjugate Comprising a Linker and a Drug

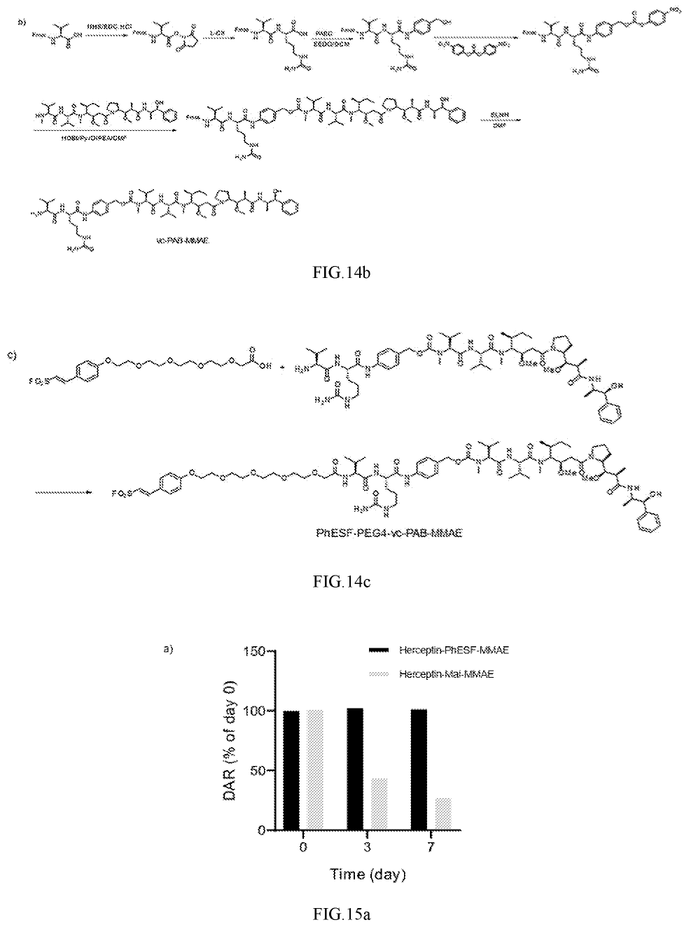

The Michael acceptor 1 was used as a linker to synthesize an ADC. In order to increase the solubility of the Michael acceptor 1, a polyethylene glycol was modified on the Michael acceptor 1 (FIG. 14a), then the toxin MMAE was conjugated to the cleavable vc-PAB (FIG. 14b), and finally a condensation reaction was conducted to obtain a ADC: PhESF-PEG4-MMAE (FIG. 14c).

### Example 8 Producing an ADC

### 8.1 DAR=3.2

The PhESF-PEG4-MMMAE obtained in example 7 was used to react with the reduced antibody in order to synthesize an ADC (Herceptin-PhESF-PEG4-MMAE) with a DAR of about 3.2. As a comparative example, an ADC (Herceptin-Mal-MMAE) with a DAR of about 3.2 was also synthesized.

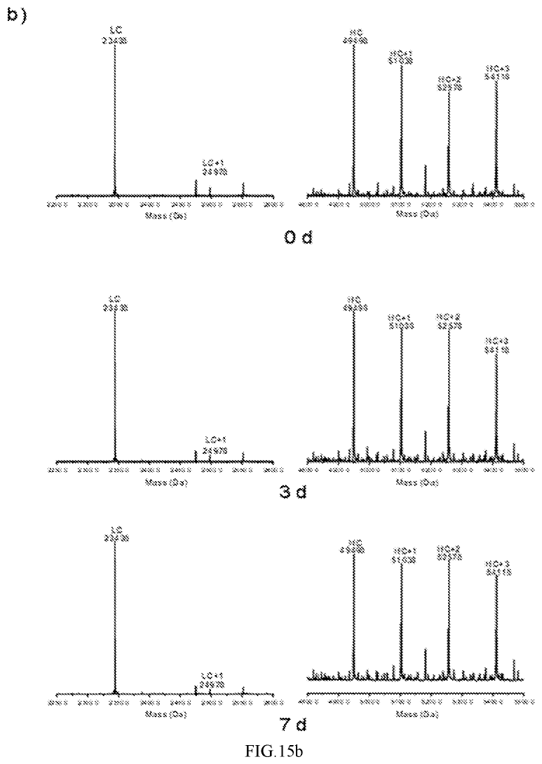

The stability of the two ADC in the serum was firstly investigated, and it was found that the DAR value of the ADC with the Michael acceptor 1 as the linker did not significantly decrease after 7 days, while the DAR value of the ADC with the maleimide decreased 70% (FIGS. 15a-15c).

FIGS. 15a-15c show the result of ADC stability test. FIG. 15a shows the result of a ADC stability test in human serum; FIG. 15b shows the Mass spectrometry results of Herceptin-PhESF-PEG4-MMAE at different time points in serum; FIG. 15c shows the Mass spectrometry results of Herceptin-PhESF-PEG4-MMAE at different time points in serum. [0354] 8.2 DAR=3.8

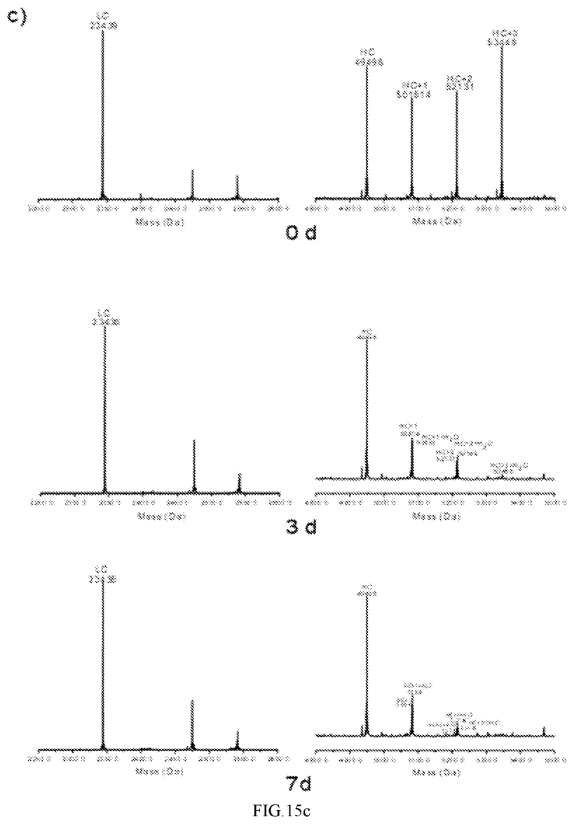

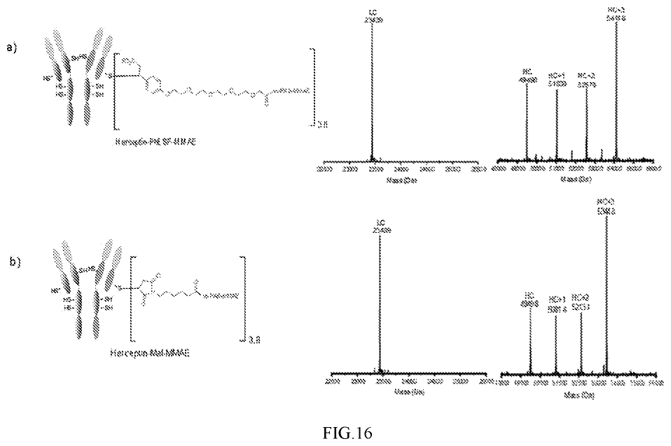

The DAR value of ADC with MMAE as the drug generally does not exceed 4. In order to achieve an enhanced killing effect, ADC with a DAR value of about 3.8 was synthesized and verified with the Mass spectrometry (FIGS. 16a-16b). As a comparative example, an ADC (Herceptin-Mal-MMAE) with a DAR of about 3.8 was also synthesized.

FIG. 16a shows the structure and the Mass spectrometry results of Herceptin-PhESF-PEG4-MMAE; FIG. 16b shows the structure and the Mass spectrometry results of Herceptin-Mal-MMAE.

### Example 9 Tumor Cell Killing Test

Two ADCs (Herceptin-PhESF-PEG4-MMAE and Herceptin-Mal-MMAE prepared in example 8) were used for a tumor cell killing test, it can be seen from the results that the two ADCs have similar killing efficiency to HER2⁺ cells SKBR3, NCI-N87, MDA-MB-435 (FIGS. 17a-17c)

The IC₅₀ value of the Herceptin-PhESF-PEG4-MMAE to the three cells were 18.3 ng/mL, 7.76 ng/mL, 14.66 ng/mL, respectively, and the IC₅₀ value of the Herceptin-Mal-MMAE were 24.48 ng/mL, 10.94 ng/mL, and 12.03/mL, respectively.

To the HER-cell MDA-MB-231 and the HER-cell MDA-MB-435, the two ADCs showed no obvious killing effects (FIGS. 17d-17e).

It was found that the Herceptin-Mal-MMAE was able to significantly kill HER2-cells at high concentrations, while Herceptin-PhESF-PEG4-MMAE showed no obvious killing at high concentrations (FIG. 17f).

Then the bystander lethality test of ADC was conducted, and it was found that the stable Michael acceptor 1 did not affect its bystander lethality (FIG. 17g).

FIGS. 17a-17h show the results of the tumor cell killing test. FIG. 17a shows the killing result to the SKBR3 cell, FIG. 17b shows the killing result to the NCI-N87 cell; FIG. 17c shows the killing result to the HER*MDA-MB-435 cell; FIG. 17d shows the killing result to the HER-MDA-MB-435 cell; FIG. 17e shows the killing result to the MDA-MB-231 cell; FIG. 17f shows the killing result of a relatively high concentration of ADC to the MDA-MB-231 cell; FIG. 17g shows the result of an ADC bystander killing test and FIG. 17h shows the killing result of a 10 ug/mL concentration of ADC to the MDA-MB-231 cell.

### Example 10 Chemical Synthesis and Analyze

### Example 10.1 Synthesis of SSF-PEG4-PAB-MMAE 1

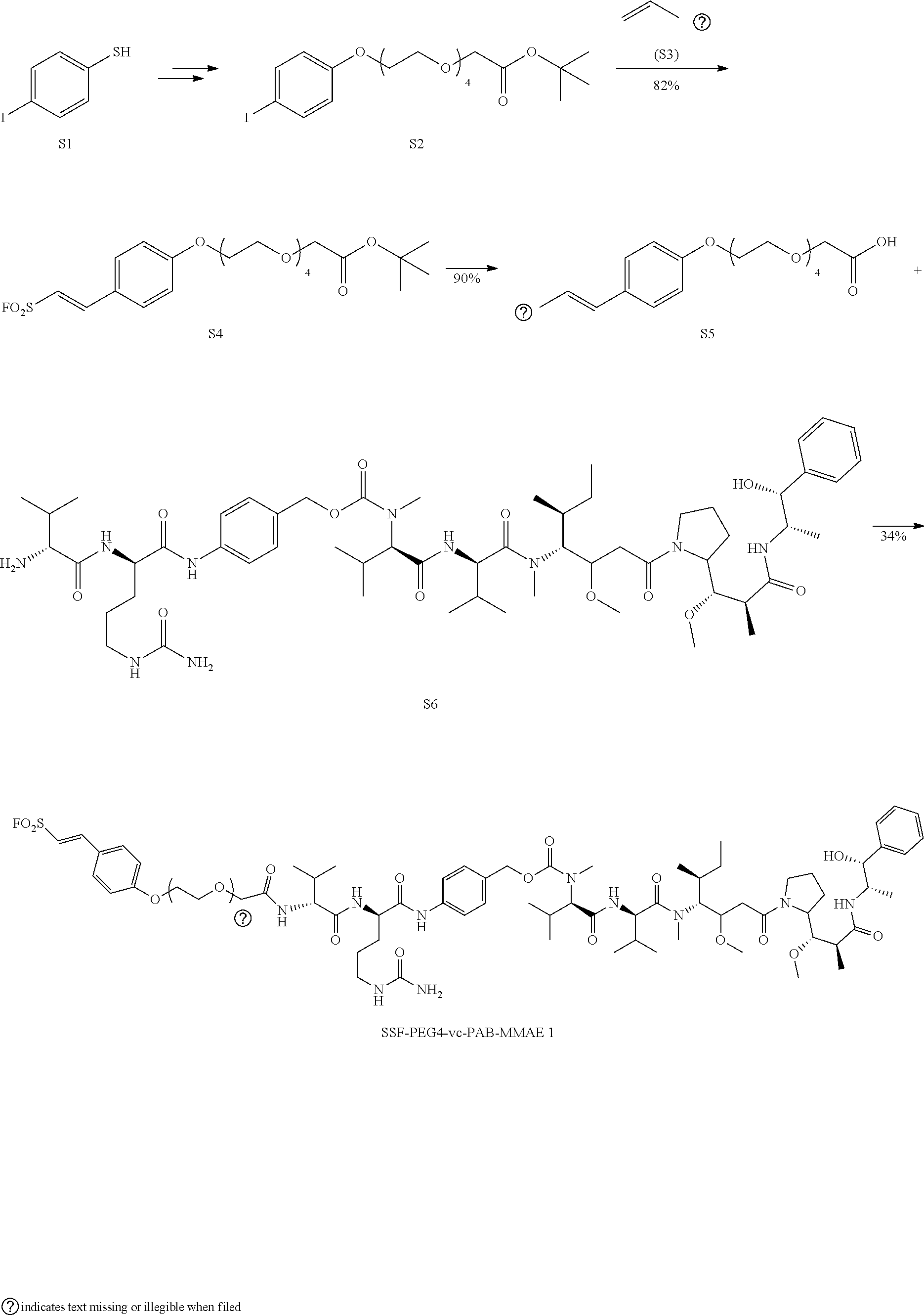

Compound S2 was synthesized based on previously published procedures. ¹H NMR(400 MHz, CDCl₃): δ 7.52 (2H, d, J=8.8 Hz), 6.68 (2H, d, J=8.8 Hz), 4.07 (2H, t, J=4.6 Hz), 4.00 (2 H, s), 3.83 (2H, t, J=4.6 Hz), 3.70-3.65 (12 H, in), 1.46 (9 H, s); ¹³C NMR (100 MHz, CDCl₃): δ 169.68, 158.69, 138.17, 117.06, 82.92, 81.58, 70.83, 70.71, 70.60, 69.60, 69.03, 67.53, 28.12. HRMS m/z (ESI): calcd for C₂₀H₃₁IO₇ [M+H]⁺: 511.1193, found 511.1192.

tert-butyl(E)-14-(4-(2-(fluorosulfonyl)vinyl)phenoxy)-3,6,9,12-tetraoxatetradecanoate (Compound S4):

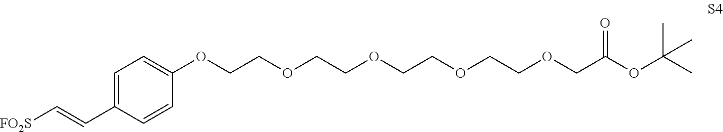

An oven-dried reaction tube (20 mL) charged with AgTFA (2.4 mmol, 1.2 equiv), Pd(OAc)₂ (22 mg, 5 mol %), acetone (5 mL), S2 (1.02 g, 2 mmol) and ethene S3(440 mg, 4.0 mmol, 2 equiv) was added. The resulting mixture was refluxed at 60° C. for 12 h. The crude material was purified by column chromatography on silica gel to give S4. (403 mg, 82%). ¹H NMR (400 MHz, CDCl₃): δ 7.71 (1H, d, J=15.2 Hz), 7.48 (2H, d, J=8.8 Hz), 6.97 (2H, d, J=8.8 Hz), 6.70 (1 H, dd, J =15.2 Hz, 2.4 Hz), 4.17 (2H, t, J=4.6 Hz), 4.00 (2 H, s), 3.86 (2H, t, J=4.6 Hz), 3.72-3.64 (12 H, m), 1.46 (9 H, s); ¹³C NMR (100 MHz, CDCl₃): 169.65, 162.50, 148.60, 131.08, 123.68, 115.43, 114.82, 114.54, 81.59, 70.87, 70.70, 70.58, 69.45, 69.00, 67.75, 28.09; ¹⁹F (376 MHz, CDCl₃): δ +63.01; HRMS m/z (ESI): calcd for C₂₂H₃₃FO₉S [M+H]⁺:493.1908, found 493.1904.

(E)-14-(4-(2-(fluorosulfonyl)vinyl)phenoxy)-3,6,9,12-tetraoxatetradecanoic acid (Compound S5)

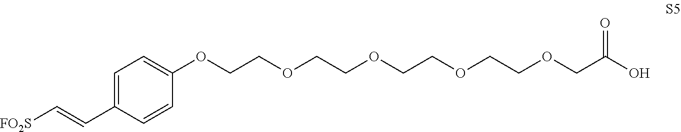

An oven-dried reaction tube (20 mL) charged with S4 (286 mg, 0.5 mmol), TFA (2 mL), and CH₂Cl₂ (2 mL) was added. The resulting mixture was incubated at rt for 4 h. The crude material was purified by column chromatography on silica gel to give S5 (192 mg, 90%).¹H NMR (400 MHz, CDCl₃): δ 7.73 (1H, d, J=15.2 Hz), 7.49 (2H, d, J=8.8 Hz), 6.98 (2H, d, J=8.8 Hz), 6.70 (1 H, dd, J=15.2 Hz, 2.4 Hz), 4.19 (2H, t, J=4.6 Hz),4.11 (2 H, s), 3.89 (2H, t, J=4.6 Hz), 3.75-3.71 (4H, m), 3.68-3.65 (8H, m); ¹³C NMR (100 MHz, CDCl₃): 171.77, 162.38, 148.61, 131.10, 123.77, 115.46, 114.87, 114.59, 71.13, 70.90, 70.58, 70.35, 70.23, 70.15, 69.44, 69.17, 67.53; ¹⁹F (376 MHz, CDCl₃): δ +63.00; HRMS m/z (ESI): calcd for C₁₈H₂₅FO₉S [M−H]⁻: 435.1125, found 435.1138.

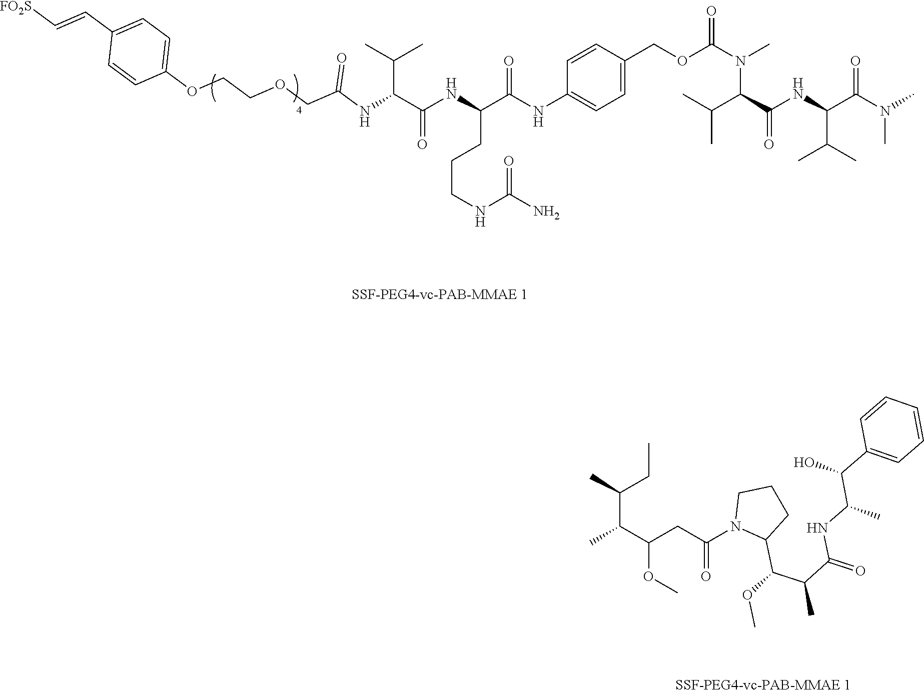

An oven-dried reaction tube (20 mL) charged with EDC (0.1 mmol, 2 eq), DIPEA (0.15 mmol, 3 eq), dry DMF (2 mL), S5 (22 mg, 0.05 mmol, 1 eq), and S6 (67 mg, 0.06 mmol, 1.2 eq) was added. The resulting mixture was incubated at rt for 12 h. The crude product was purified by preparative HPLC to give 6 (26 mg, 34%). HRMS m/z (ESI): calcd for C₇₆H₁₁₇FN₁₀O₂₀S [M+H]⁺: 1541.8229, found 1541.8230. The product 1 was analysed by LC-MS.

LC-MS chromatograms and mass spectrum of 1

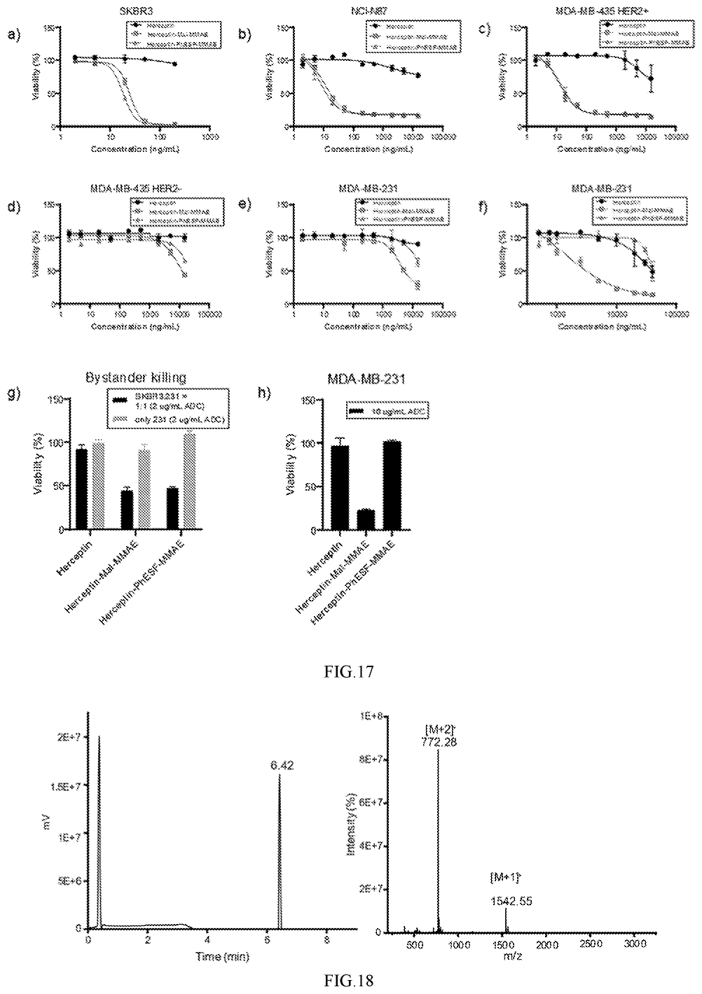

The results were shown in FIG. 18.

### Example 10.2 Synthesis of SSF-PEG4-GGFG-Dxd 3

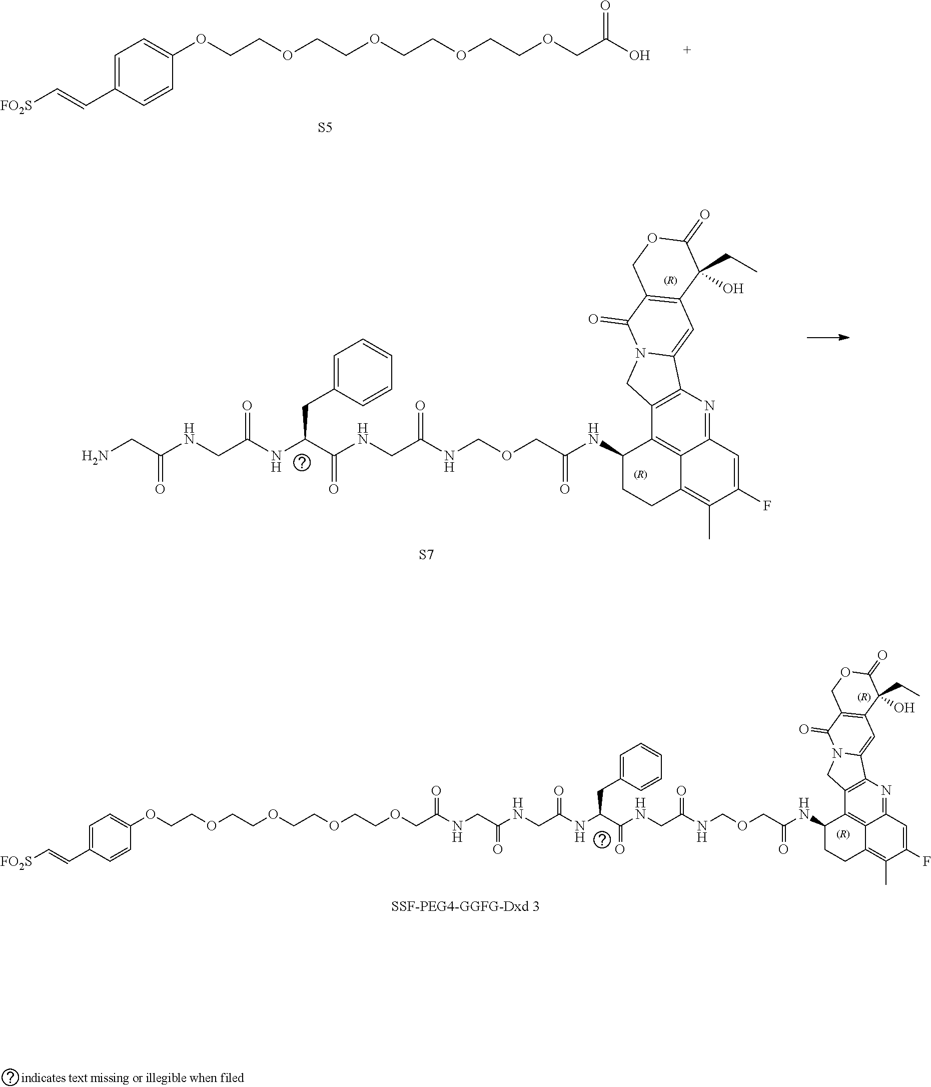

(E)-2-(4-(((S)-10-benzyl-1-(((1R,9R)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-1,6,9,12,15,18-hexaoxo-3,20,23,26,29-pentaoxa-5,8,11,14,17-pentaazahentriacontan-31-yl)oxy)phenyl)ethene-1-sulfonyl fluoride (Compound SSF-PEG4-GGFG-Dxd 3)

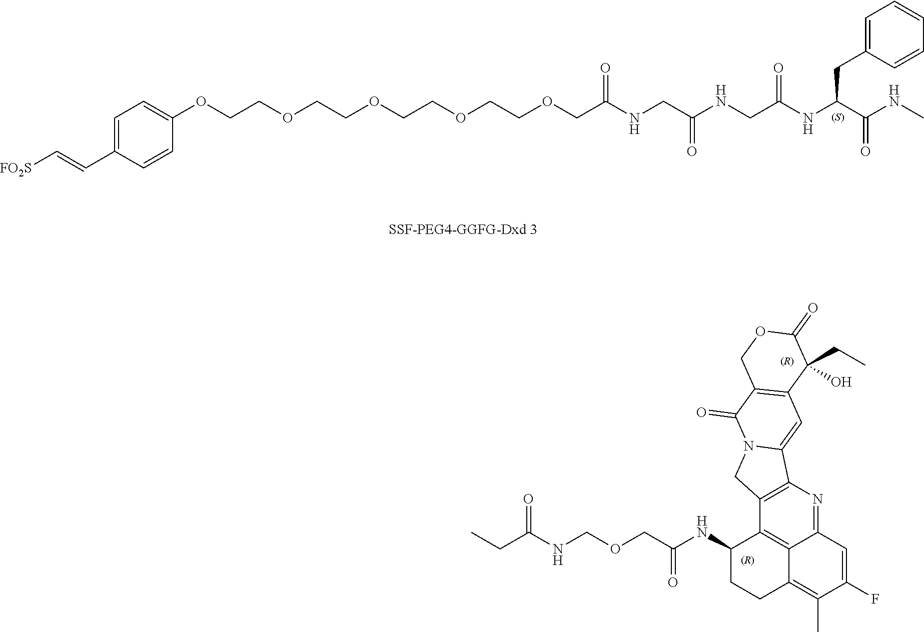

An oven-dried reaction tube (20 mL) charged with EDC (0.04 mmol, 2 eq), DIPEA (0.06 mmol, 3 eq), dry DMF (2 mL), S7 (16.8 mg, 0.02 mmol, 1 eq), and S5 (10.5 mg, 0.024 mmol, 1.2 eq) was added. The resulting mixture was incubated at rt for 12 h. The crude product was purified by preparative HPLC to give 3 (8.0 mg, 32%). HRMS m/z (ESI): calcd for C₇₆H₁₁₇FN₁₀O₂₀S [M+Na]⁺: 1281.4238, found 1281.4196. The product 3 was analysed by LC-MS.

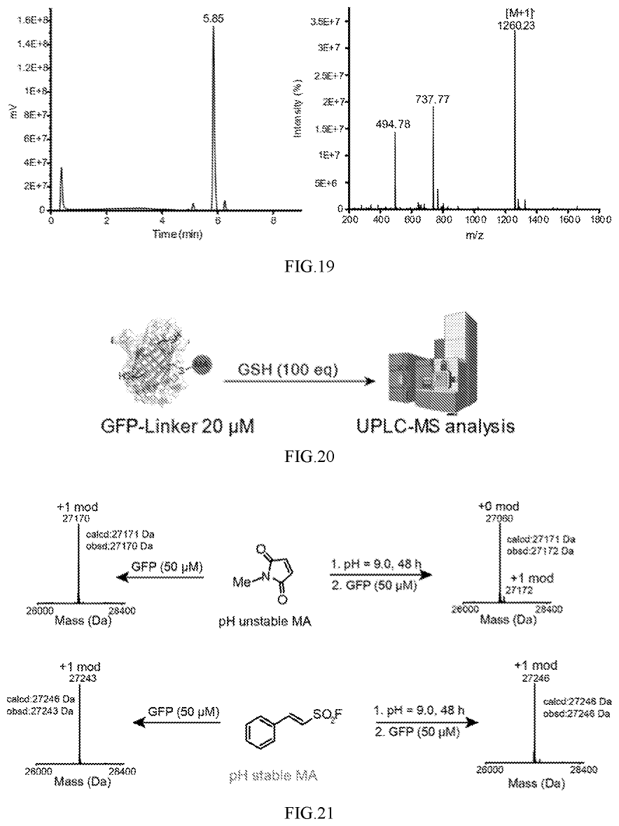

The results were shown in FIG. 19.

### Example 10.3 Synthesis of MA 16-Biotin

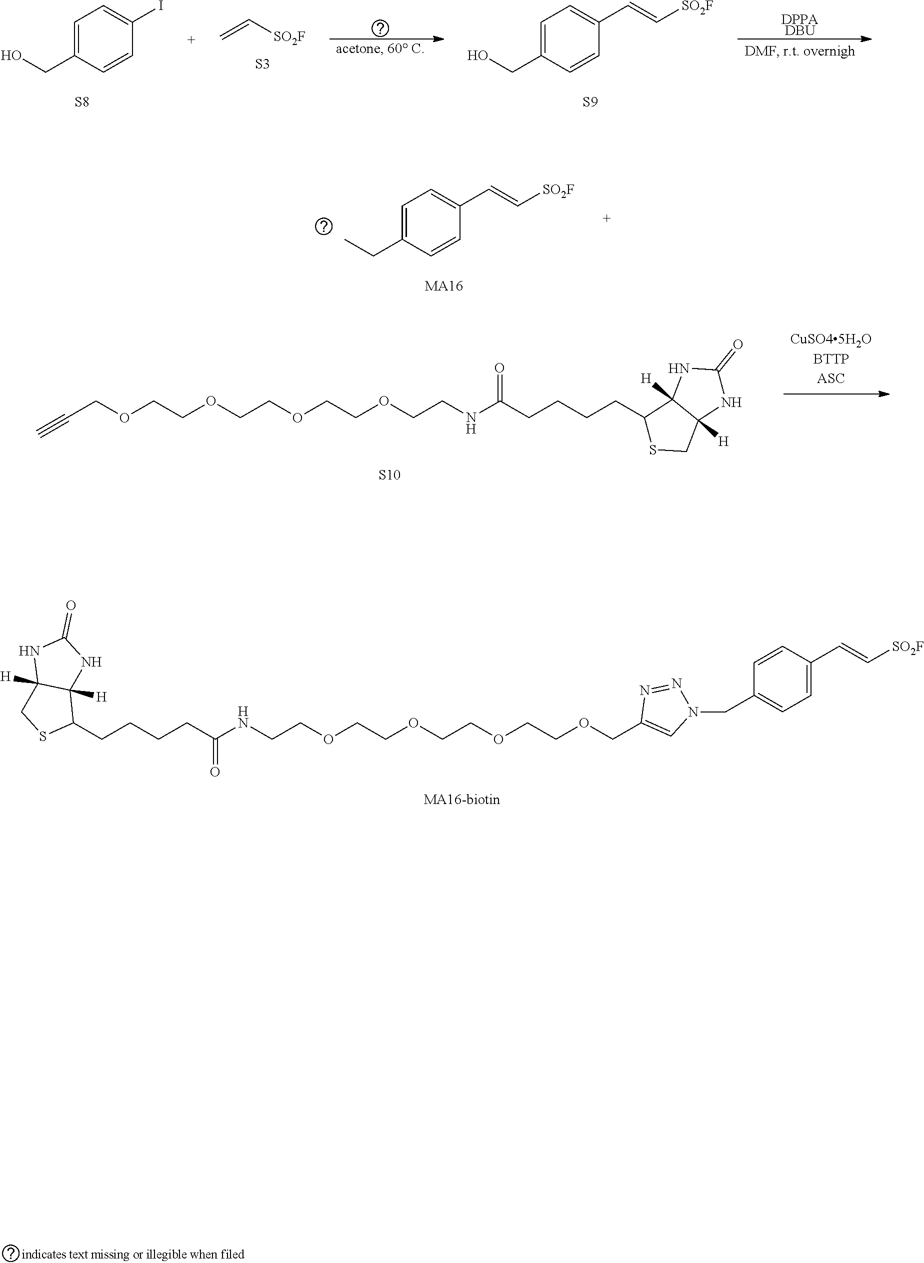

(E)-2-(4-(azidomethyl)phenyl)ethene-1-sulfonyl fluoride (Compound MA 16):

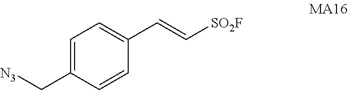 [SMILES: [N-]=[N+]=NCc1ccc(/C=C/S(=O)(=O)F)cc1]

To a solution of S9 (432 mg, 2 mmol, 1 eq.) in anhydrous DMF (2 mL), diphenyl phosphoryl azide (DPPA) (2 mmol, 1 eq.) was added dropwisely under inert atmosphere. The mixture was cooled to 0° C., and diazabicycloundecene (DBU) (2 mmol, 1 eq.) was added dropwisely. The resulting mixture was incubated at rt for 12 h and quenched with water. The solution was extracted with DCM. The organic layers were combined with brine, dried over Na₂SO₄, filtered, and concentrated under reduced pressure. The crude material was purified by column chromatography on silica gel to give MA 16 (120 mg, 25%). ¹H NMR (400 MHz, CDCl₃): δ 7.81 (1H, d, J=15.4 Hz), 7.58 (2H, d, J=6.8 Hz), 7.43 (2H, d, J=6.8 Hz), 6.88 (1 H, dd, J=15.4 Hz, 2.8 Hz), 4.43 (2 H, s); ¹³C NMR (100 MHz, CDCl₃): 148.01, 140.31, 129.48, 128.93, 118.61, 118.33, 54.16; ¹⁹F (376 MHz, CDCl₃): δ +62.32; HRMS m/z (ESI): calcd for C₉H₈FN₃O₂S [M+H]⁺: 242.0400, found 242.0402.

(E)-2-(4-((4-(15-oxo-19-((3aS,6aR)-2-oxohexahydro-1H-thieno[3,4-d]imidazol-4-yl)-2,5,8,11-tetraoxa-14-azanonadecyl)-1H-1,2,3-triazol-1-yl)methyl)phenyl)ethene-1-sulfonyl fluoride (Compound MA 16-biotin)

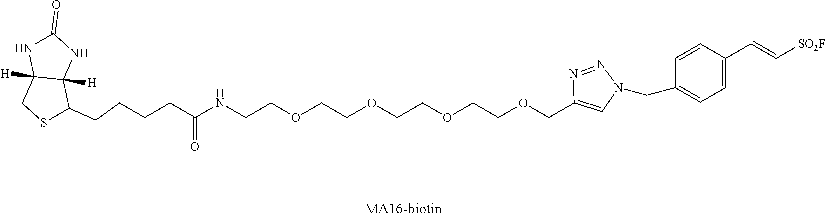 [SMILES: O=C(CCCCC1SC[C@@H]2NC(=O)N[C@H]12)NCCOCCOCCOCCOCc1cn(Cc2ccc(/C=C/S(=O)(=O)F)cc2)nn1]

An oven-dried reaction tube (20 mL) charged with CuSO4 5H₂O (0.005 mmol, 0.1 eq), BTTP (0.01 mmol, 0.2 eq), ASC (0.02 mmol, 0.4 eq), DMSO (1.5 mL), H₂O (0.5 mL) and S10 (0.055 mmol, 1.1 eq) and MA 16 (0.05 mol, 1 eq) was added. The resulting mixture was reacted at 37° C. for 12 h. The crude material was purified by column chromatography on silica gel to give MA 16-biotin (31 mg, 89%). ¹H NMR (400 MHz, CDCl₃): δ 7.80 (1 H, s), 7.58 (1H, d, J=15.2 Hz), 7.45 (1H, d, J=8.0 Hz), 7.19-7.13 (3H, m), 5.40 (2H, s), 4.40 (2H, s), 4.22-4.19 (1H, m), 4.03-4.00 (1H, m), 3.38-3.33 (4H, m), 3.31-3.28 (6H, m), 3.23-3.20 (2H, m), 3.05-3.00 (3H, m), 2.92-2.87 (1H, m), 2.64-2.60 (1H, m), 2.42-2.39 (1H, m), 1.91 (2H, t, J=7.4 Hz), 1.41-1.28 (4H, m), 1.16-1.10 (2H, m); ¹³C NMR (100 MHz, CDCl₃): 174.83, 164.69, 147.83, 139.64, 131.68, 129.65, 128.57, 119.23, 118.95, 70.12, 70.01, 70.07, 69.82, 69.52, 69.12, 63.43, 62.10, 60.39, 55.57, 53.15, 39.61, 38.98, 35.28, 28.33, 28.06, 25.43; ¹⁹F (376 M1 Hz, CDCl₃): δ +59.81; HRMS m/z (ESI): calcd for C₃₀H₄₃FN₆O₈S₂ [M+H]⁺: 699.2946, found 699.2942.

### Example 10.4 Synthesis of SSF-NHS

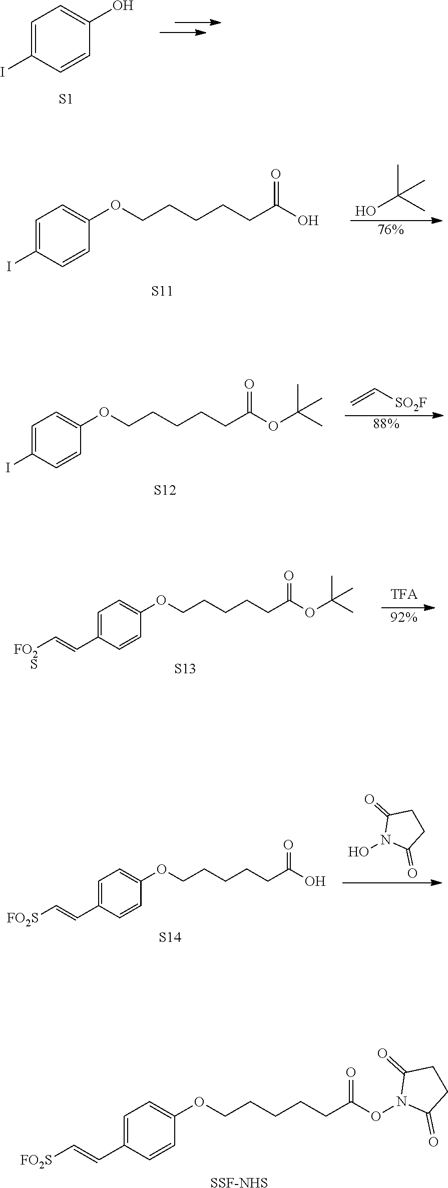

Compound S10 was synthesized based on previously published procedures. 1 H NMR (400 MHz, CDCl₃): δ 7.54 (2H, d, J=8.9 Hz), 6.66 (2H, d, J=8.9 Hz), 3.92 (2H, t, J=6.4 Hz), 2.40 (2H, t, J=7.4 Hz), 1.83-1.74 (2H, m), 1.73-1.67 (2H, m), 1.56-1.78 (2H, m); HRMS m/z (ESI): calcd for C₁₂H₁₅IO₃ [M−H]⁻: 332.9988, found 332.9985.

tert-butyl 6-(4-iodophenoxy)hexanoate (Compound S 12):

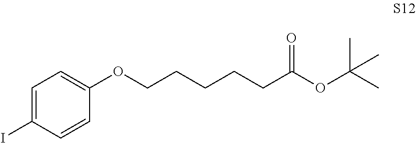 [SMILES: CC(C)(C)OC(=O)CCCCCOc1ccc(I)cc1]

An oven-dried reaction tube (20 mL) charged with S11 (501 mg, 1.5 mmol, 1 eq.), tBuOH (5 eq.), anhydrous DCM (2 mL) was added. The mixture was cooled to 0° C., DMAP (0.1 eq.) was added dropwisely. Then DCC(1.1 eq) was add. The resulting mixture was stirred at rt for overnight. The crude material was purified by column chromatography on silica gel to give S12. (444.6 mg, 76%).¹H NMR (400 MHz, CDCl₃): δ 7.53 (2H, d, J=8.9 Hz), 6.66 (2H, d, J=8.9 Hz), 3.91 (2 H, t, J=6.4 Hz), 2.24 (2H, t, J=7.4 Hz), 1.81-1.74 (2H, m), 1.68-1.60 (2H, m), 1.51-1.46 (2 H, m), 1.44 (9 H, s); HRMS m/z (ESI): calcd for C₁₆H₂₃IO₃ [M+H]⁺: 391.0770, found 391.0773.

tert-butyl (E)-6-(4-(2-(fluorosulfonyl)vinyl)phenoxy)hexanoate (Compound S13):

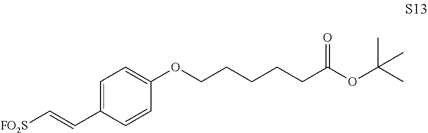

An oven-dried reaction tube (20 mL) charged with AgTFA (0.6 mmol, 1.2 equiv), Pd(OAc)₂ (5.5 mg, 5 mol %), acetone (2 mL), S12 (1.02 g, 0.5 mmol) and ethene S3 (220 mg, 1 mmol, 2 eq) was added. The resulting mixture was refluxed at 60° C. for 12 h. The crude material was purified by column chromatography on silica gel to give S13. (163.7 mg, 88%).¹H NMR (400 MHz, CDCl₃): δ 7.74 (1H, d, J=15.4 Hz), 7.49 (2H, d, J=8.8 Hz) 6.93 (2H, d, J=8.8 Hz), 6.68 (1 H, dd, J=2.8 Hz), 4.02 (2H, t, J=6.4 Hz), 2.25 (2H, t, J=7.4 Hz), 1.86-1.79 (2H, m), 1.70-1.63 (2H, m), 1.54-1.51 (2H, m), 1.44 (9 H, s); ¹⁹F (376 MHz, CDCl₃): δ +63.07; HRMS m/z (ESI): calcd for C₁₈H₂₅FO₅S [M+H]⁺: 373.1485, found 373.1486.

(E)-6-(4-(2-(fluorosulfonyl)vinyl)phenoxy)hexanoic acid (Compound S14):

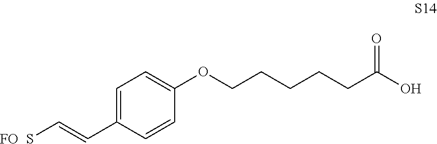

An oven-dried reaction tube (20 mL) charged with S12 (93 mg, 0.25 mmol), TFA (2 mL), and DCM (2 mL) was added. The resulting mixture was stirred at rt for 4 h. The crude material was purified by column chromatography on silica gel to give S13 (72 mg, 92%).1 H NMR (400 MHz, CDCl₃): δ 7.74 (1H, d, J=15.4 Hz), 7.49 (2H, d, J=8.8 Hz) 6.94 (2H, d, J=8.8 Hz), 6.69 (1 H, dd, J=2.6 Hz), 4.02 (2H, t, J=6.4 Hz), 2.41 (2H, t, J=7.4 Hz), 1.91-1.81 (2H, m), 1.77-1.69 (2H, m), 1.58-1.52 (2H, m); ¹⁹F (376 MHz, CDCl₃): δ +63.05; HRMS m/z (ESI): calcd for C₁₄H₁₇FO₅S [M−H]⁻: 315.0702, found 315.0703.

2,5-dioxopyrrolidin-1-yl (E)-6-(4-(2-(fluorosulfonyl)vinyl)phenoxy)hexanoate (Compound SSF-NHS)

An oven-dried reaction tube (20 mL) charged with S14 (63 mg, 0.2 mmol), N-Hydroxy succinimide (1.2 eq), EDC (1.2 eq) and CH₂Cl₂ (4 mL) was added. The resulting mixture was stirred at rt for 12 h. After completion, the reaction was quenched with 20 mL water, and extracted 3 times with 20 mL DCM. The combined organic phases were dried over anhydrous Na₂SO₄ and concentrated to give SSF-NHS (67 mg, 81%). ¹H NMR (400 MHz, CDCl₃): δ 7.74 (1H, d, J=15.4 Hz), 7.49 (2H, d, J=8.8 Hz) 6.94 (2H, d, J=8.8 Hz), 6.69 (1 H, dd, J=15.4 Hz 2.6 Hz), 4.03 (2H, t, J=6.4 Hz), 2.84 (4 H, s), 2.66 (2H, t, J=7.4 Hz), 1.88-1.82 (4H, m), 1.64-1.59 (2 H, m); ¹³C NMR (100 MHz, CDCl₃): 169.17, 168.46, 162.70, 148.68, 131.12, 123.48, 115.31, 114.35, 67.87, 33.96, 30.84, 28.54, 25.60, 24.29; ¹⁹F (376 MHz, CDCl₃): δ +63.06; HRMS m/z (ESI): calcd for C₁₈H₂₀FNO₇S [M+H]⁺: 436.0842, found 436.0827.

### Example 10.5 Synthesis of SSF-OSO₂F

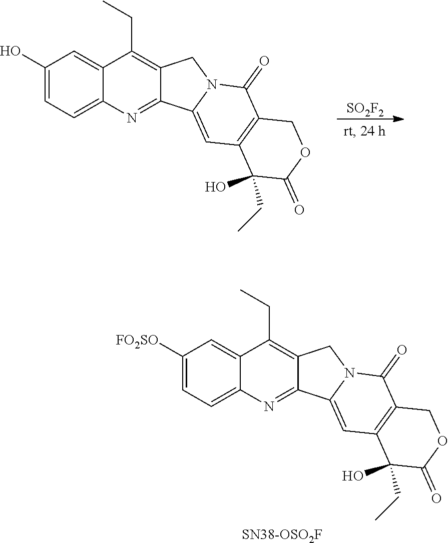

An oven-dried reaction tube (20 mL) charged with SN38 (98.3 mg, 0.25 mmol), Et3N (3 eq), and DCM (2 mL) was added. The mixture was stirred at room temperature for 24 h under SO₂F₂ balloon. The reaction was quenched by addition of brine (30 mL) and extracted with DCM (30 mL). The combined organic layer was dried over anhydrous Na₂SO₄, filtered and concentrated under reduced pressure. The residue was purified by column chromatography to obtain SN38-0.0F (100.5 mg, 85%) as a yellow foam. 1 H NMR (400 MHz, DMSO): δ 8.37 (1H, d, J=2.6 Hz), 8.33 (1H, d, J=9.4 Hz) 7.66 (1 H, s) 5.60 (1H, d, J=16.4 Hz), 5.41-5.37 (3H, m), 3.29-3.27 (2H, m), 1.99-1.94 (2H, m), 1.43 (3H, t, J=7.6 Hz), 1.01 (3H, t, J=7.4 Hz).

### In Vitro Cytotoxicity Studies

Cells were seeded in a 96-well plate at 5,000 cells per well for 24 h at 37° C. with 5% CO₂. Serial dilutions of SN38, SN38—OSO₂F were added to the cells in complete growth medium and incubated at 37° C. with 5% CO₂ for 96 h. Cell viability was evaluated using a Cell Counting-Lite 2.0 Luminescent Cell Viability Assay (Vazyme, DD1101-01). Cell viability was plotted as a percentage of untreated cells. Each measurement was taken in triplicate.

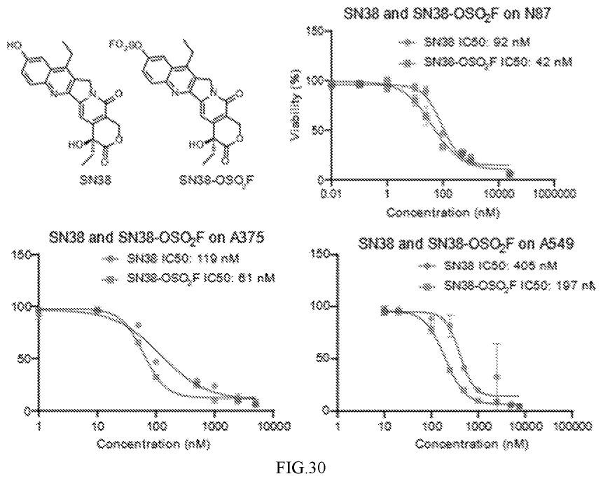

FIG. 30 shows the results of Cell viability assays with cell lines (N87) for assessing SN38, SN38—OSO₂F.

### Example 10.7 Synthesis of SSF-PEG4-T785

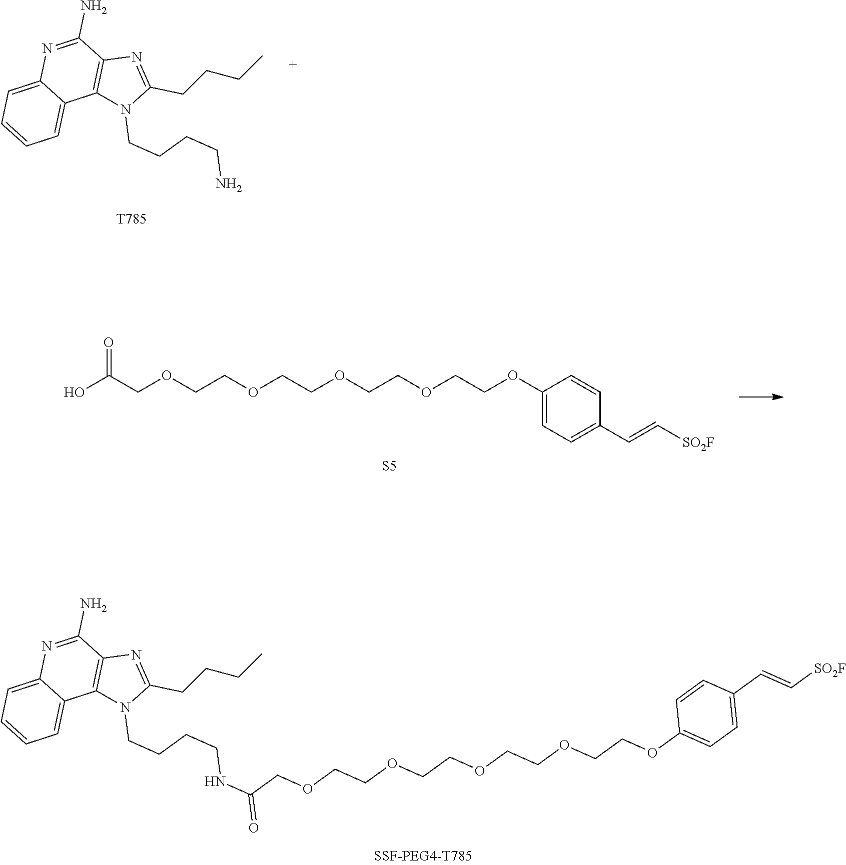 [SMILES: CCCCc1nc2c(N)nc3ccccc3c2n1CCCCN ; "T785" ; O=C(O)COCCOCCOCCOCCOc1ccc(/C=C/S(=O)(=O)F)cc1 ; "S5" ; CCCCc1nc2c(N)nc3ccccc3c2n1CCCCNC(=O)COCCOCCOCCOCCOc1ccc(/C=C/S(=O)(=O)F)cc1]

An oven-dried reaction tube (20 mL) charged with EDC (0.1 mmol, 2 eq), DIPEA (0.15 mmol, 3 eq), dry DMF (2 mL), T785 (15.6 mg, 0.05 mmol, 1 eq), and S5 (26 mg, 0.06 mmol, 1.2 eq) was added. The resulting mixture was incubated at rt for 12 h. The crude product was purified by preparative HPLC to give SSF-PEG4-T785 (15.6 mg, 24%). HRMS m/z (ESI): calcd for C₃₆H₄₈FN₅O₈S [M+H]⁺: 730.3286, found 730.3253.

### Example 11 Synthesis of antibody-biotin conjugate

### Example 11.1 Synthesis of trastuzumab-MA 16-Cy5.5 conjugate

Cysteine Selective Protein Modification with MA 16

MA 16 (1 μL, 20 mM in DMS0) and PBS (15 μL, pH=7.5, 50 mM) were added to a solution of protein (4 μL, 250 μM, in PBS buffer). After incubation at 37° C. for 2 h, the solution was desalted to give the product before LC-MS analysis.

### Reduction of Antibodies

TCEP.HCl (2.6 μL, 25 mM in PBS) was added to a solution of antibodies (KN026, KN046, trastuzumab) (40 μL, 160 μM in PBS), and the resulting solution was incubated at 37° C. for 2 h. The solution was desalted to give reduced antibodies before being subjected to LC-MS analysis.

MA 16-biotin (1.6 μL, 5 mM in DMSO) and PBS (34.5 μL, pH=7.4, 50 mM) were added to a solution of antibodies (4 μL, 100 μM, in PBS buffer). After incubation at 37° C. for 2 h, the solution was desalted to obtain antibody biotin conjugates before LC-MS analysis.

### Synthesis of Trastuzumab-MA 16-Cy5.5 Conjugate

MA 16 (1.6 μL, 5 mM in DMSO) and PBS (34.5 μL, pH=7.4, 50 mM) were added to a solution of trastuzumab (4 μL, 100 μM, in PBS buffer). After incubation at 37° C. for 2 h, the solution was desalted to obtain trastuzumab-MA 16.

DBCO-Cy5.5 (0.8 μL, 5 mM in DMSO) and PBS (15 μL, pH=7.4, 50 mM) were added to a solution of trastuzuma-MA 16 (4 μL, 50 μM, in PBS buffer). After incubation at 37° C. for 12 h, the solution was desalted to obtain trastuzumab-MA 16-Cy5.5 before LC-MS analysis.

### SDS-PAGE Analysis of Antibody Conjugates

Two microliters of trastuzumb, trastuzumb-MA 16-Cy5.5 and trastuzumb-MA 16-biotin were mixed with 10 μL of ultrapure water and 4 μL SDS-PAGE loading buffer containing 2-mercaptoethanol. Samples were heated to 95° C. or 10 min and completely loaded onto SDS-PAGE gels.

### Example 11.2 Synthesis of Trastuzumab-1 (Herceptin-PhESF-MMAE)

1 (i.e. SSF-PEG4-PAB-MMAE 2.6 μL, 25 mM in DMSO), DMSO (17.4 μL), and PBS (280 μL, pH=7.4, 50 mM) were added to a solution of trastuzumab (100 μL, 130 μM, PBS buffer). After incubation at 25° C. for 2 h, the solution was desalted to obtain trastuzumab-1.

### Example 11.3 Synthesis of Trastuzumab-2 (Herceptin-Mal-MMAE)

2 (i.e. Mal-vc-PAB-MMAE 1.8 μL, 25 mM in DMSO), DMSO (18.2 μL), and PBS (280 μL, pH=7.4, 50 mM) were added to a solution of trastuzumab (100 μL, 130 μM, PBS buffer). After incubation at 25° C. for 2 h, the solution was desalted to obtain trastuzumab-2. Mal-vc-PAB-MMAE was purchased from SHANGDONG MEITAI PHARM. CO., LTD.

### Example 11.4 Synthesis of Trastuzumab-3 (Herceptin-PhESF-Dxd)

3 (i.e. SSF-PEG4-GGFG-Dxd 1.5 μL, 25 mM in DMSO), DMSO (0.5 μL), and PBS (10 μL, pH=9.0, 50 mM) were added to a solution of trastuzumab (10 μL, 130 μM, PBS buffer). After incubation at 37° C. for 12 h, the solution was desalted to obtain trastuzumab-3.

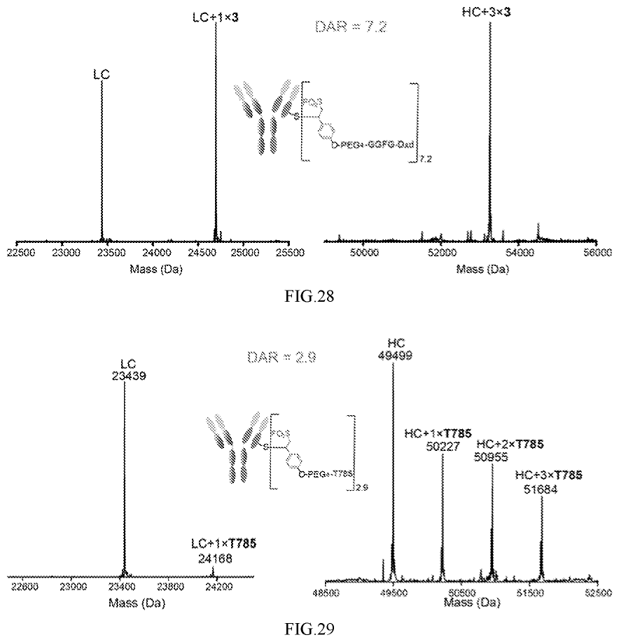

FIG. 28 shows the deconvoluted intact protein MS of trastuzumab-3.

### Example 11.5 Synthesis of Trastuzumab-T785

SSF-PEG4-T785 (13 μL, 10 mM in DMSO), DMSO (27 μL), and PBS (560 μL, pH=9.0, 50 mM) were added to a solution of trastuzumab (200 μL, 130 μM, PBS buffer). After incubation at 25° C. for 2 h, the solution was desalted to obtain trastuzumab-T785.

FIG. 29 shows the deconvoluted intact protein MS of trastuzumab-T785.

### Example 12 Analysis of MA 16 Related Conjugate

### SDS-PAGE Analysis of Antibody Conjugates

Two microliters of trastuzumb, trastuzumb-MA 16-Cy5.5 and trastuzumb-MA 16-biotin were mixed with 10 μL of ultrapure water and 4 μL SDS-PAGE loading buffer containing 2-mercaptoethanol. Samples were heated to 95° C. or 10 min and completely loaded onto SDS-PAGE gels.

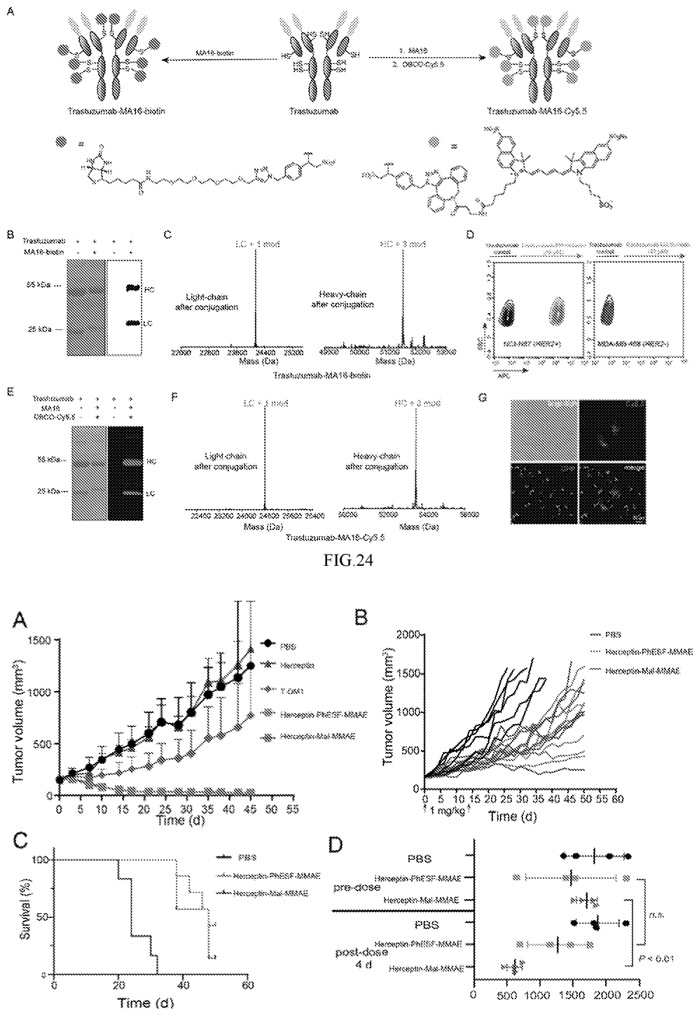

The results of trastuzumab modification with detection probes using MA 16 derivatives are shown in FIG. 24.

FIG. 24A shows the synthetic scheme for the attachment of MA16-biotin and MA16-Cy5.5 to trastuzumab. Reaction conditions of trastuzumab-MA16-biotin: 20 μM trastuzumab, 400 μM MA16-biotin, PBS, 37° C., 2 h. Reaction conditions of trastuzumab-MA16: 20 μM trastuzumab, 400 μM MA16, PBS, 37° C., 2 h. Reaction conditions of trastuzumab-MA16-Cy5.5: 10 μM trastuzumab-MA16, 200 μM DBCO-Cy5.5, PBS, 37° C., 12 h. FIG. 24B and FIG. 24C show the SDS-PAGE gel, western blot and LC-MS analysis of trastuzumab before and after the reaction with MA16-biotin.

### Flow Cytometry Assay

Cells were suspended in flow cytometry buffer (PBS with 2% FBS) containing trastuzumab-MA 16-biotin or control trastuzumab and incubated at 4° C. for 45 minutes. After washing with flow cytometry buffer twice, the cells were further incubated with APC-streptavidin (BioLegend, 405207) at 4° C. for 30 minutes, resuspended and washed with flow cytometry buffer another two times, and analyzed using an Agilent flow cytometer (Angilent NovoCyte Quanteon). Isotype control antibody staining was used to define gates for positive and negative cells. Agilent Novoexpress was used for all flow data analysis.

FIG. 24D shows the flow cytometry analysis of trastuzumab-MA16-biotin-stained cancer cells. NCI-N87 (HER2+) and MDA-MB-468 cells (HER2-) were incubated with trastuzumab-MA16-biotin, while the control groups were treated with trastuzumab. After staining, cells were further stained with SA-APC to detect biotin.

FIG. 24E and FIG. 24F show the analysis of trastuzumab before and after modification with Cy5.5 via fluorescence imaging (right), Coomassie staining (left) and LC-MS.

### Fluorescence Imaging

MDA-MB-231 and NCI-N87 cells were grown on sterile glass cover slips or slides overnight at 37° C. After washing briefly with DPBS, sections were incubated with 20 μg/mL trastuzumab-MA 16-Cy5.5 for 1 hour at 37° C. MDA-MB-231 cells were transfected with a plasmid encoding GFP. Cocultured cells were then imaged under a microscope at a magnification of 40×.

FIG. 24G shows the flourescence imaging of trastuzumab-MA16-Cy5.5 enabled specific cell surface HER2 detection (scale bar 50 μm).

### Example 13 Analysis of Trastuzumab-Related Conjugate

### Example 13.1 Stability Studies in Human

In an Eppendorf tube, 90 μL human serum was mixed with 10 μL trastuzumab-1 (20 mg/ml) or trastuzumab-2 for each sample individually to give a final solution of 0.2 mg/mL ADC in human serum. Samples were incubated in human mouse at 37° C. for 3 and 7 days. Samples were incubated in mouse serum at 37° C. for 3 days. Samples for day 0 were directly processed further.

### Example 13.2 In Vitro Cytotoxicity Studies

Cells were seeded in a 96-well plate at 5,000 cells per well for 24 h at 37° C. with 5% CO₂. Serial dilutions of trastuzumab-1, trastuzumab-2 and trastuzumab were added to the cells in complete growth medium and incubated at 37° C. with 5% CO₂ for 96 h. Cell viability was evaluated using a Cell Counting-Lite 2.0 Luminescent Cell Viability Assay (Vazyme, DD1101-01). Cell viability was plotted as a percentage of untreated cells. Each measurement was taken in triplicate.

### Example 13.3 Bystander Killing Assay

SKBR-3 and MDA-MB-231 cell mixtures were seeded in a 96-well plate at 1:1 per well, and MDA-MB-231 cells alone were seeded in 96-well plates at the same density for 24 h at 37° C. with 5% CO₂. Then, 2 μg/mL trastuzumab, trastuzumab-1 and trastuzumab-2 were added and incubated at 37° C. with 5% CO₂ for 96 h. MDA-MB-231 was a stable cell line that overexpress luciferase. Cell viability was evaluated by Dual Luciferase Reporter Assay Kit (Vazyme, DL101-01).

### Example 13.4 Nude Mouse Xenograft Assay

All in vivo studies were performed in accordance with the local guidelines of the Institutional Animal Care and Use Committee (Approval No: IACUC-2101001). NCI-N87 cells (2 million) were inoculated subcutaneously into specific pathogen-free female nude mice. The tumor-bearing mice were randomized into treatment and control groups. When the average volume of tumors reached approximately 100 to 200 mm³, dosing was initiated on day 0. Each substance was administered intravenously to the mice, 1 mg/kg trastuzumab-1 or trastuzumab-2 as well as vehicle (PBS) on days 0 and 14. Tumor volume was defined as 1/2* length * width², and tumor size was recorded every three days.

The results were shown in FIG. 25.

FIG. 25A shows the antitumor activity of ADCs (5 mg/kg) in an NCI-N87 tumor xenograft model in BALB/c nude mice. FIG. 25B shows the antitumor activity of ADCs (1 mg/kg) in an NCI-N87 tumor xenograft model in BALB/c nude mice. Tumor volumes of the seven mice per group are shown separately. FIG. 25C shows the Kaplan-Meier survival analysis of the study shown in FIG. 25B. FIG. 25D shows that Neutropenia observed in rats following a 20 mg/kg ADC dose. Four animals were dosed with trastuzumab-1, trastuzumab-2 or vehicle and sampled for hematology markers.

### Example 13.5 Safety Studies

All in vivo studies were performed in accordance with the local guidelines of the Institutional Animal Care and Use Committee (Approval No:ZJCLA-IACUC-20040026). ADCs or PBS were dosed intravenously in 12- to 14-week-old female rats at 20 mg/kg (four rats per dose group, randomly assigned). Prior to dosing and at post-dose days, 4 serum samples were taken for hematology analysis.

### Example 14 Analysis of MA 2-Related Conjugate

### Hydrolytic Stability of MA 2 vs MA 5

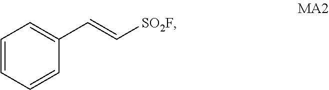 [SMILES: O=S(=O)(F)/C=C/c1ccccc1]

5 mM final concentration), or MA 5 (i.e.

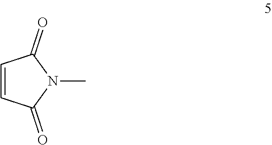

5 mM final concentration) in 100 μL of 50 mM PBS pH 9.0 was shaken for 48 h at 37° C. for 48 h. Then 1 μL the reaction mixture and PBS (15 μL, pH=7.4, 50 mM) were added to a solution of GFP (4 μL, 250 μM, HEPES buffer), respectively. After incubation at 37° C. for 2 h, the solution was desalted to give the product before LC-MS analysis.

The Hydrolytic stability results were shown in FIG. 21.

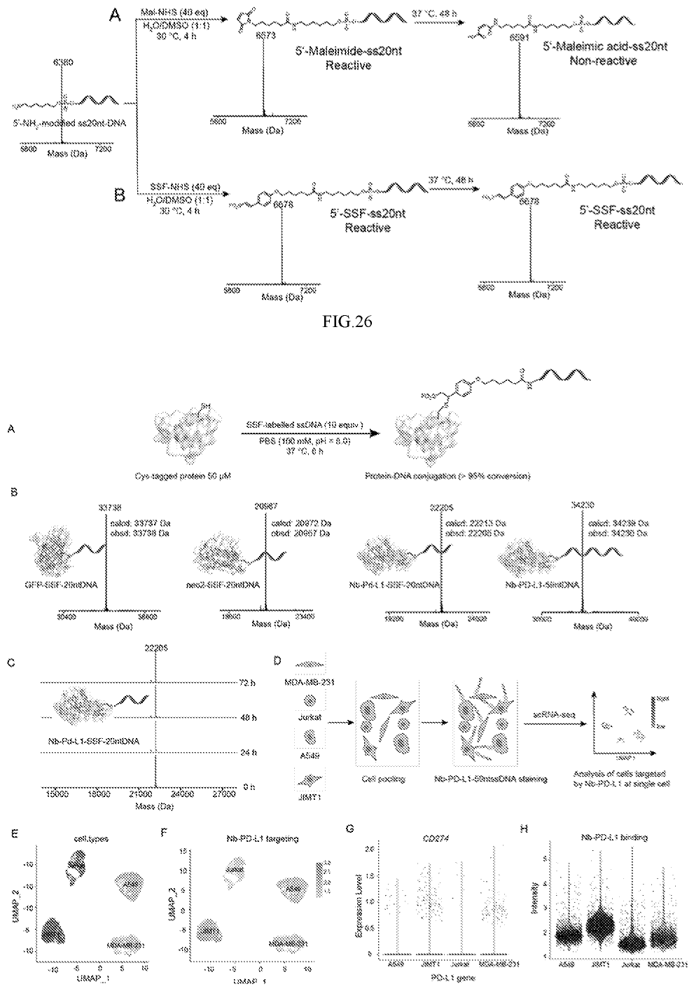

The results of stability of SSF (MA 2) versus maleimide in aqueous buffer were shown in FIG. 26.

FIG. 26A shows that 5′-malemide-ssDNA was shaken for 48 h at 37° C. Then, the reaction mixture was analyzed by LC-MS. Complete hydrolysis of 5′-malemide-ssDNA to 5′-malemic acid-ssDNA. FIG. 26B shows the hydrolytic stability of SSF: 5′-malemide-ssDNA was shaken for 48 h at 37° C. Then, the reaction mixture was analyzed by LC-MS and only the starting material was obtained.

### MA 2 Analogues Modification of GFP-TEV

MA 2 or MA 2 analogues (i.e. Compounds 1a, 1a-1, 1b, 1b-1) (1 μL, 20 mM in DMSO) and PBS (15 μL, pH=7.4, 50 mM) were added to a solution of GFP-TEV (4 μL, 250 μM in HEPES buffer). After incubation at 37° C. for 2 h, the solution was desalted to give the product before LC-MS analysis.

### Kinetic Experiment

MA 2 or previous stable linker (i.e. Compounds 2, 3, 6, 7) (1 μL, 5 mM in DMSO) or PBS (15 μL, pH=7.4, 50 mM) was added to a solution of GFP (4 μL, 250 μM, HEPES buffer). After incubation at 37° C. for various times (5 min, 10 min, 30 min, 60 min, 90 min, 120 min and 240 min), the solution was desalted to give the product before LC-MS analysis.

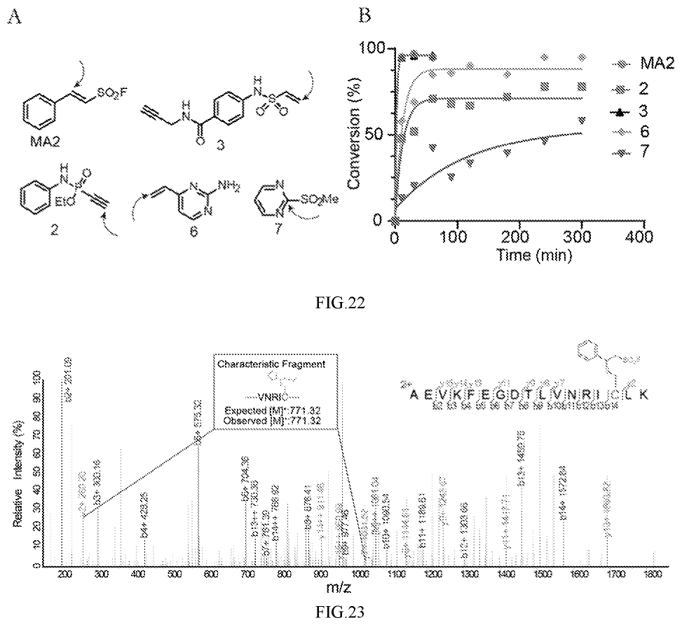

The results were shown in FIG. 22. FIG. 22 compares MA 2 with previously reported stable Cys-specific labeling reagents.

FIG. 22A shows the chemical structures of reported stable Cys-specific labeling reagents. The arrows point to the cysteine reaction sites. FIG. 22B shows the reaction kinetics of GFP (50 μM) with 5 equiv. labeling reagents.

### Competitive Experiment

MA 2 (0.5 μL, 20 mM in DMSO), 2-5 (0.5 μL, 20 mM in DMSO), and PBS (15 μL, pH=7.4, 50 mM) were added to a solution of GFP (4 μL, 250 μM in HEPES buffer). After incubation at 37° C. for 2 h, the solution was desalted to give the product before LC-MS analysis.

The result of MS/MS spectra of GFP fragment modified with MA 2 is shown in FIG. 23.

FIG. 24 showed the Cys-specific modification using SSF on different proteins.

FIG. 24A shows the reaction scheme for IA2 with different proteins. FIG. 24B shows the deconvoluted intact protein MS of the protein-MA6 conjugates, including neo2, Nb-PD-LI, GFP, KN046, trastuzumab, KN026.

### Determination of Trastuizumab-MA 2 Conjugate Binding Affinity

SKBR3 cells were allowed to bind with various concentrations of trastuzumab-MA6 or trastuzumab in 200 μL flow cytometry buffer (PBS with 2% FBS) on ice for 30 minutes. After binding, the cells were washed twice with PBS and further incubated with trastuzumab-Cy5.5 on ice for 30 minutes, resuspended and washed with flow cytometry buffer another two times, and analyzed using Agilent flow cytometer (Angilent NovoCyte Quanteon).

### Example 15 LC-MS/SDS-PAGE Based Conjugated Integrity Study of Nb-PD-L1-20ntssDNA in the Presence of 10% Human Serum

Nb-PD-L1-20ntssDNA (2 μL, 50 μM), human serum (2 μL) and PBS (16 μL, pH=8.0) the mixture was incubated at 37° C. in the dark for 24 h, 48 h, 72 h. The reaction mixture was analyzed by LC-MS and SDS-PAGE. SDS-PAGE: ten microliters of Nb-PD-L1-20nt ssDNA was mixed with 5 μL SDS-PAGE loading buffer containing 2-mercaptoethanol. Samples were heated to 95° C. for 10 min and completely loaded onto SDS-PAGE gels.

FIG. 27 shows the construction of site-specific DNA-protein conjugates by SSF-ssDNA and the application of Nb-PD-L1 ssDNA in single-cell RNA sequencing.

FIG. 27A shows the scheme of protein modification with SSF-ssDNA probes. FIG. 27B shows the deconvoluted mass spectra for DNA-protein conjugates constructed via 20nt SSF-ssDNA or 59nt SSF-ssDNA probes. FIG. 27C shows the LC-MS based conjugate integrity study of Nb-PD-L1-20ntssDNA in the presence of 10% human serum with deconvoluted mass spectra of samples taken at specified time points. FIG. 27D shows the schematic overview of Nb-PD-L1-ssDNA enabled CITE-seq for detecting targeted cells at single cell level with transcriptome. FIG. 27E shows the transcriptome-based clustering of single-cell expression profiles. Cyan: Jurkat; red: A549; green: JIMT-1; violet: MDA-MB-231. FIG. 27G shows the Relative intensity of Nb-PD-L1-ssDNA targeting superimposed on the UMAP projections shown in FIG. 27F. FIG. 27G shows the violin plot describing mRNA expression level of PD-L1 (CD274) in the four cell lines. FIG. 27H shows the violin plot describing scaled (z-score)normalized UMI counts of the 59nt-ssDNA barcode (Nb-PD-L1 binding intensity) in the four cell lines.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

1. A conjugate of formula (1): 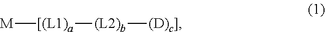
wherein
L1 is a compound of formula (I), 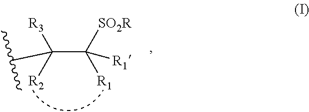
M is a biological macromolecule linked to L1 with a nucleophilic functional group of M,
L2 is a linker linked to R₁, R₃ or R₂,
D is a functional molecule,
a is an integer in a range of from 1 to 10,
b and c are independently an integer in a range of from 0 to 10, provided that b and c are not simultaneously 0,
R₁, R₂, and R₃, are independently H, an optionally substituted alkyl, or an optionally substituted aryl,
R₁, is H or the isotope thereof and
first C, linking R₁, and a second C, linking R₂, optionally form a ring.

2-3. (canceled)

4. The conjugate of claim 1, wherein M is selected from a group consisting of a protein, a DNA, a RNA, and a virus.

5-7. (canceled)

8. The conjugate of claim 1, wherein L2 is selected from the group consisting of a cleavable linker, a non-cleavable linker, a hydrophilic linker, a hydrophobic linker, a procharged linker, an uncharged linker, and a dicarboxylic acid-based linker.

9-14. (canceled)

15. The conjugate of claim 1, wherein R₁ is —H.

16. The conjugate of claim 1, wherein R₁ is —H.

17. The conjugate of claim 1, wherein R₃ is —H.

18. The conjugate of claim 1, wherein R₂ is an optionally substituted phenyl.

19. The conjugate of claim 1, wherein
R₂ is 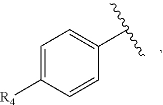
and
R₄ is selected from a group consisting of —OH, —PO₃H₂, —SeH, —SH, optionally substituted alkyl-OH, optionally substituted alkyl-halogen, optionally substituted alkyl-N₃, —B(OH)₂, -halogen, —OTf, optionally substituted alkyl-NH₂, —O-optionally substituted alkyl-C—CH, and —CO—NH—C≡CH-optionally substituted alkyl.

20. (canceled)

21. The conjugate of claim 1, wherein
R₂ is 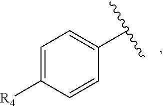
R₄ is —O—(CH₂)n₁—COO—R₅,
n₁ is an integer in a range of from 1 to 10, and
R₅ is 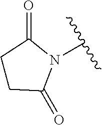 [SMILES: *N1C(=O)CCC1=O]
or H.

22. The conjugate of claim 1, wherein
R₂ is 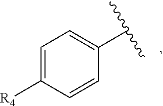
R₄ is —O—(CH₂)n₂—CO—NH—R₆,
n₂ and n, are independently an integer in a range of from 1 to 10,
R₆ is —(CH₂)n₃—CO—R₇, and
R₇ is

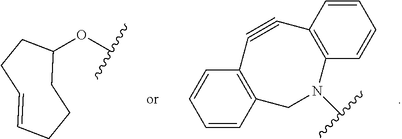 [SMILES: *OC1CC/C=C/CCC1 ; *N1Cc2ccccc2C#Cc2ccccc21]

23. The conjugate of claim 1, wherein R₂ is 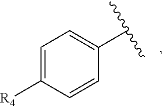
R₄ is —O—(CH₂)n₂—CO—NH—R₆,
n₂ and n₄ are independently an integer in a range of from 1 to 10,
R₆ is —(CH₂)n₄-R₈, and
R₈ is

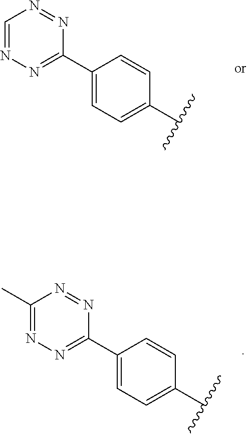 [SMILES: *c1ccc(-c2nncnn2)cc1 ; *c1ccc(-c2nnc(C)nn2)cc1]

24. The conjugate of claim 1, wherein
R₂ is 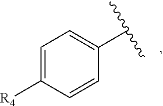
R₄ is —O—(CH₂)n₂—CO—NH—R₆,
n₂, and n₆ are independently an integer in a range of from 1 to 10, and
R₆ is —(CH₂CH₂—O)n₅—(CH₂)n₆—NH—CO—O—R₉, and R₉ is H or

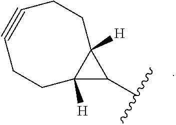 [SMILES: *C1[C@H]2CCC#CCC[C@@H]12]

25. The conjugate of claim 1, wherein
R₂ is 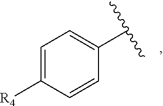
R₄ is —(OCH₂CH₂)n₇—O—(CH₂)ₙ₈—R₁₀,
n₇ and n₈ are independently an integer in a range of from 1 to 10, and
R₁₀ is COOH, —NH₂, or

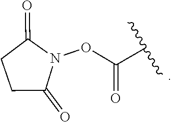 [SMILES: *C(=O)ON1C(=O)CCC1=O]

26. The conjugate of claim 1, wherein
R₂ is 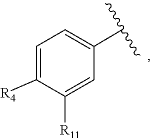
R₄ is —(OCH₂CH₂)n₇—O—(CH₂)n₈—R₁₀,
n₇ and n₈ are independently an integer in a range of from 1 to 10,
R₁₀ is COOH, —NH₂, or 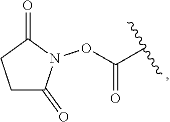 [SMILES: *C(=O)ON1C(=O)CCC1=O]
and
R₁₁ is optionally substituted alkyl-halogen, optionally substituted alkyl-N, or O optionally substituted alkyl.

27. The conjugate of claim 1, wherein R₂ is 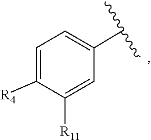
R₄ is —(OCH₂CH₂)n₇—O—(CH₂)ₙ₈—R₁₀
n₇ and n₈ are independently an integer in a range of from 1 to 10, R₁₀ is COOH, —NH₂, or 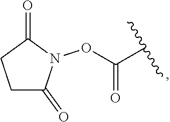 [SMILES: *C(=O)ON1C(=O)CCC1=O]
and R₁₁ is CF₃, —CN, or —OCH₃.

28. The conjugate of claim 1, wherein
R₂ is optionally substituted alkyl-CH═CH-R₁₂,
R₂ is 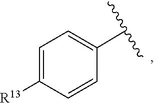
and
R₁₃ is —CH₂N₃.

29. The conjugate of claim 1, wherein the ring is an optionally substituted cycloolefin or an optionally substituted aryl-cycloolefin.

30. (canceled)

31. The conjugate of claim 1, wherein L1 is selected from the group consisting of:

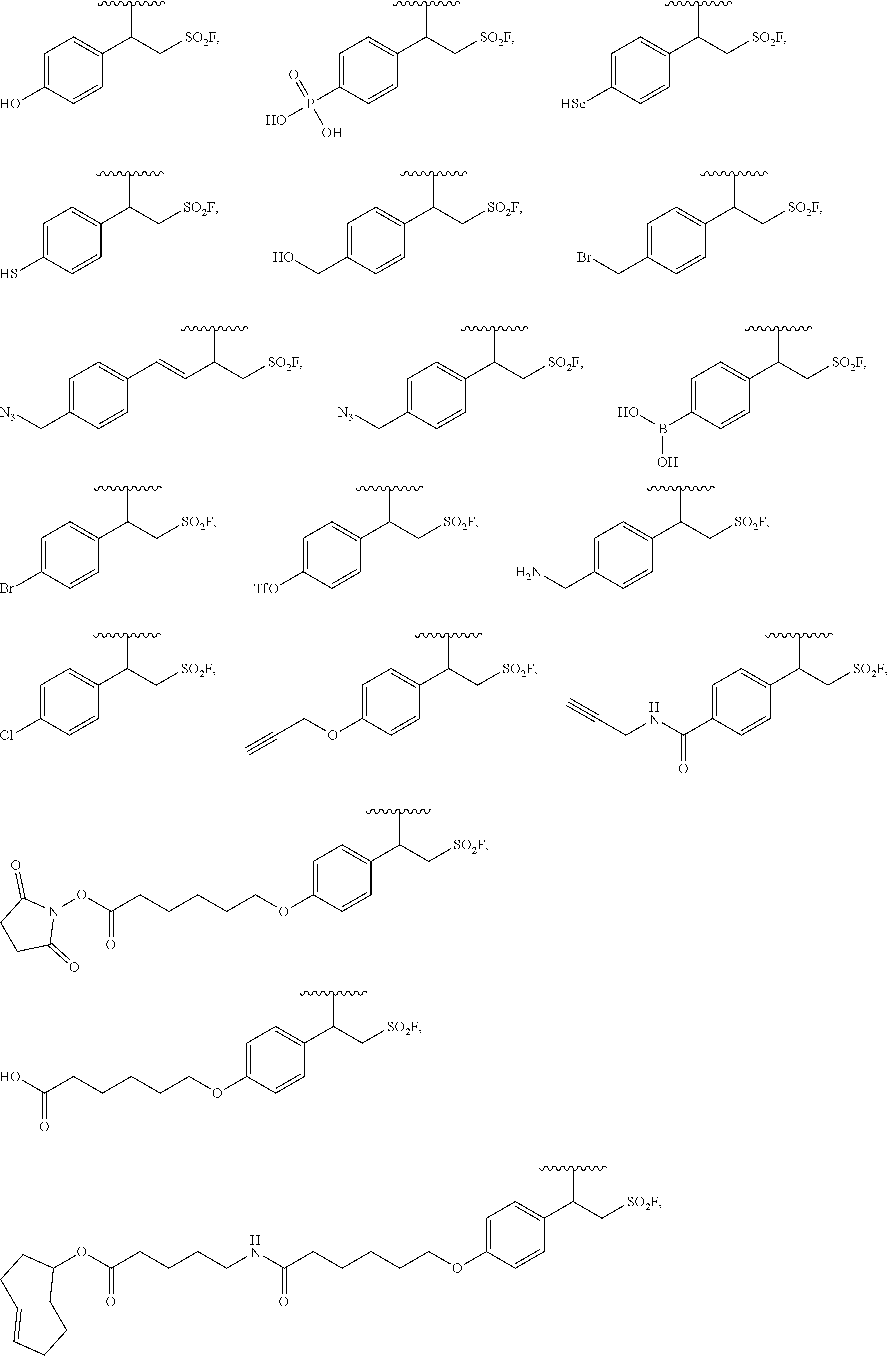

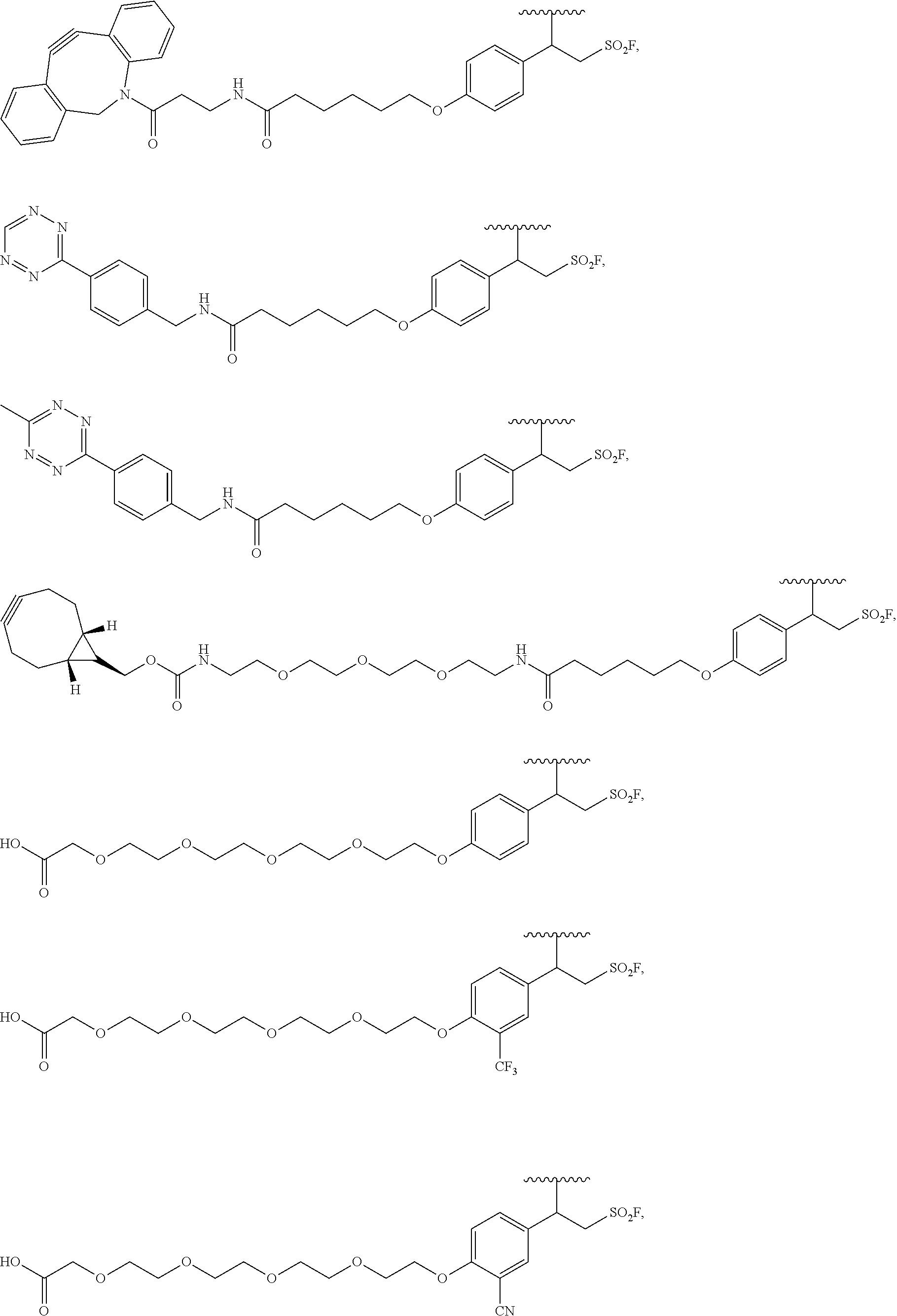

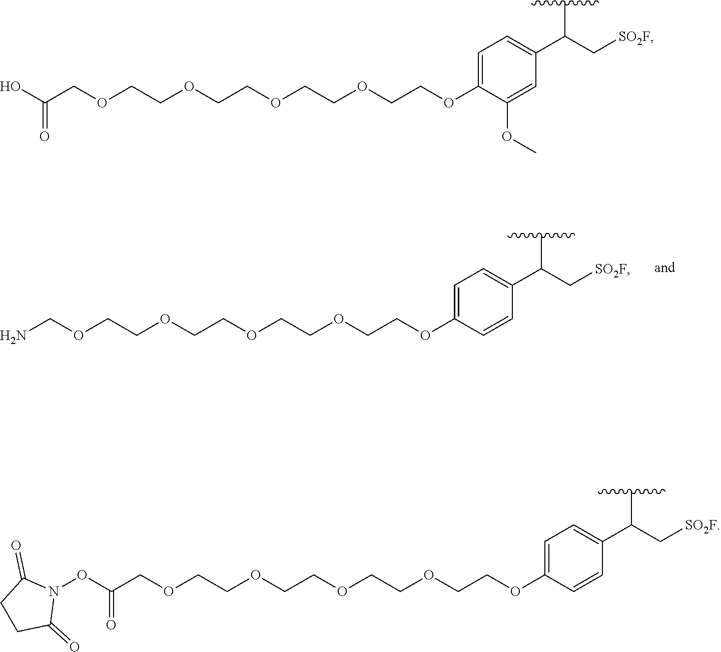 [SMILES: *C(CS(=O)(=O)F)c1ccc(OCCOCCOCCOCCOCC(=O)O)c(OC)c1 ; *C(CS(=O)(=O)F)c1ccc(OCCOCCOCCOCCOCN)cc1 ; *C(CS(=O)(=O)F)c1ccc(OCCOCCOCCOCCOCC(=O)ON2C(=O)CCC2=O)cc1]

32. The conjugate of claim 1, which is selected from the group consisting of

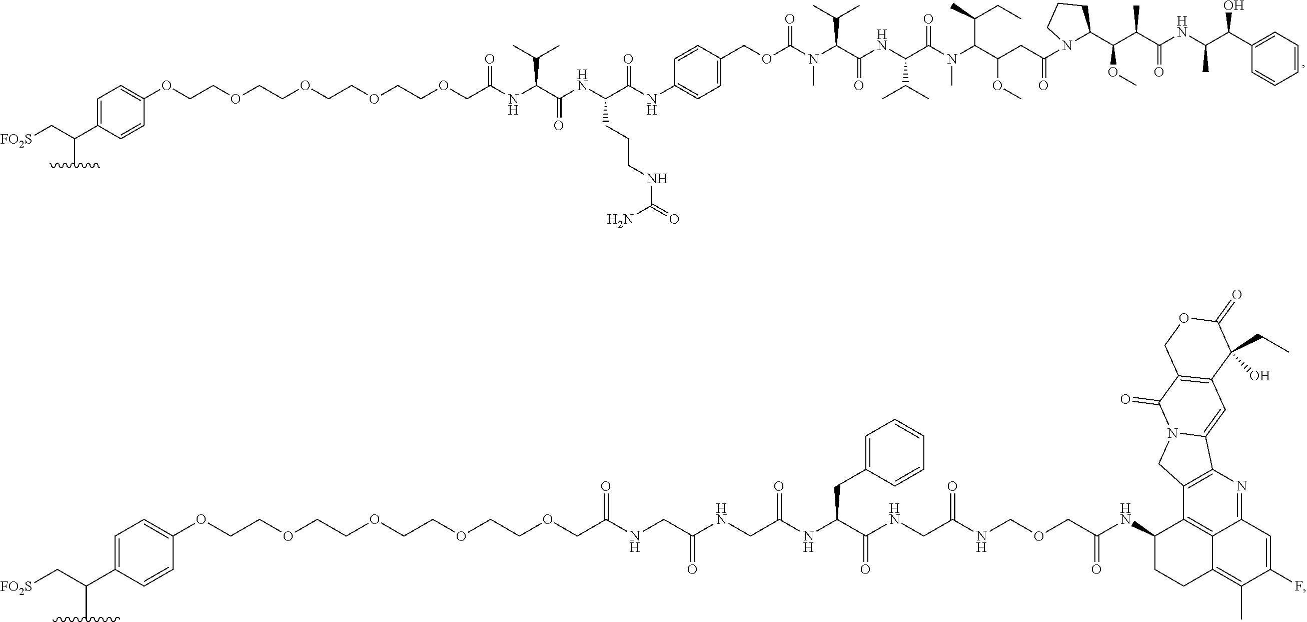

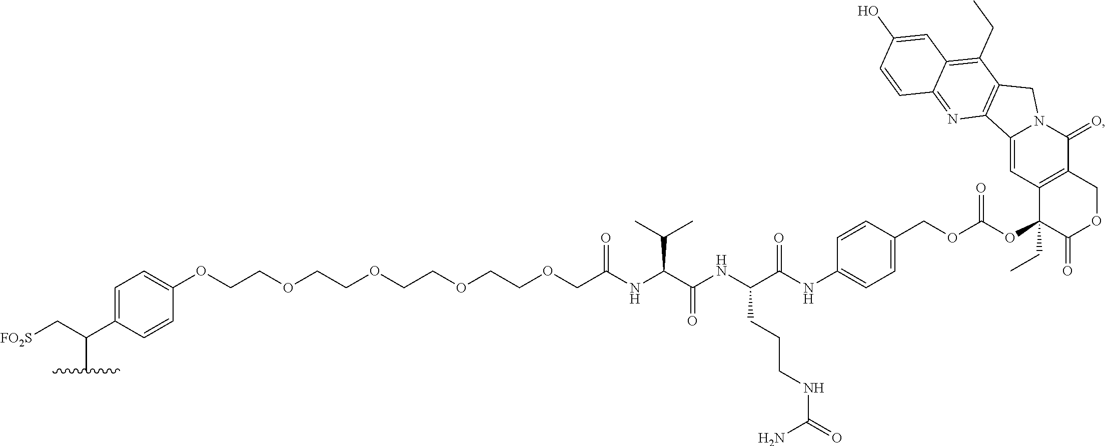

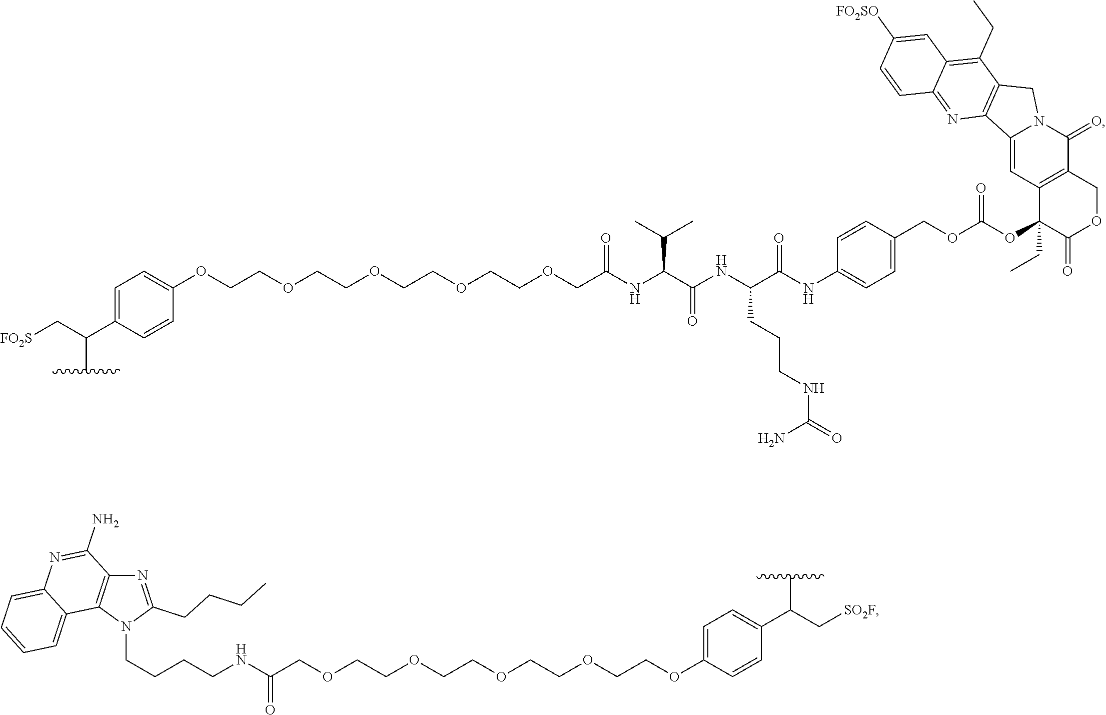

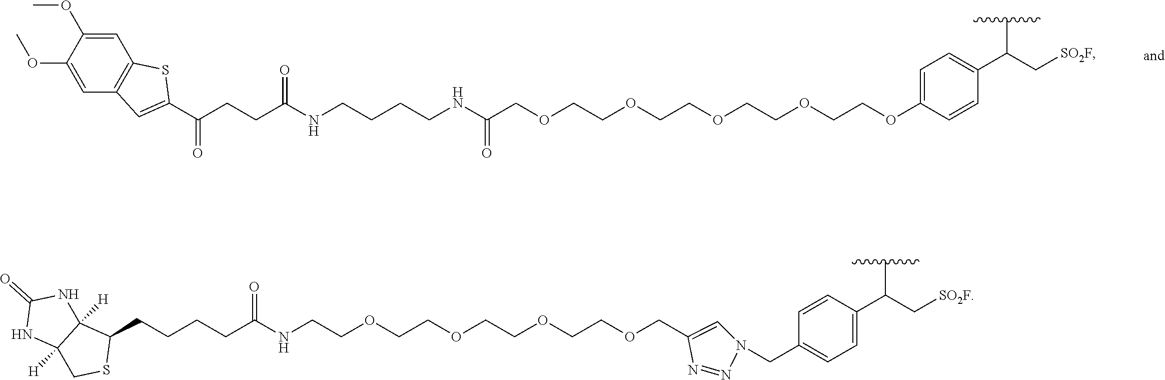 [SMILES: *C(CS(=O)(=O)F)c1ccc(OCCOCCOCCOCCOCC(=O)NCCCCNC(=O)CCC(=O)c2cc3cc(OC)c(OC)cc3s2)cc1 ; *C(CS(=O)(=O)F)c1ccc(Cn2cc(COCCOCCOCCOCCNC(=O)CCCC[C@H]3SC[C@H]4NC(=O)N[C@H]43)nn2)cc1]

33. A conjugate of formula (3): 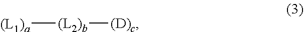
wherein L1 is a compound of formula (III), 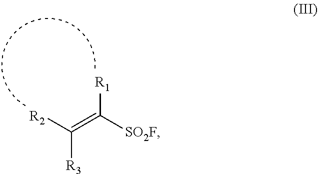
L2 is a linker linked to R₁, R₃, or R₂,
D is a functional molecule,
a is an integer in range of from 1 to 10,
b, c is each independently an integer in a range of from 0 to 10, provided that b and c are not simultaneously 0,
R¹, R², and R³ are independently H, an optionally substituted alkyl, or an optionally substituted aryl, and
a first C, linking R₁ and a second C, linking R₂, optionally form a ring.

34-94. (canceled)

95. A compound of formula (IV), (V), or (VI), or a pharmaceutically acceptable salt thereof: 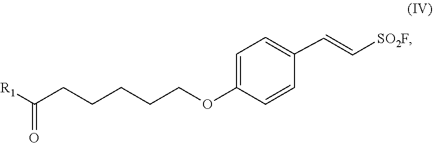
wherein
R₁ is NH—(CH₂)ₙ₁—CO—R₂, —OH, or NH—(CH₂)ₙ₂—R₃, NH—(CH₂CH₂—O)ₙ₃—(CH₂)ₙ₄—NH—CO—O—R₄, or 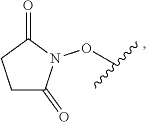 [SMILES: *ON1C(=O)CCC1=O]
n₁, n₂, n₃, and n₄ are independently an integer of 1 to 10, 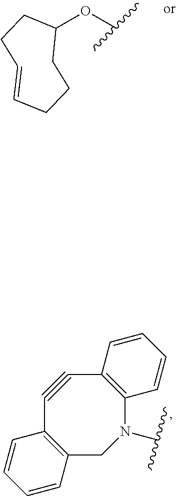 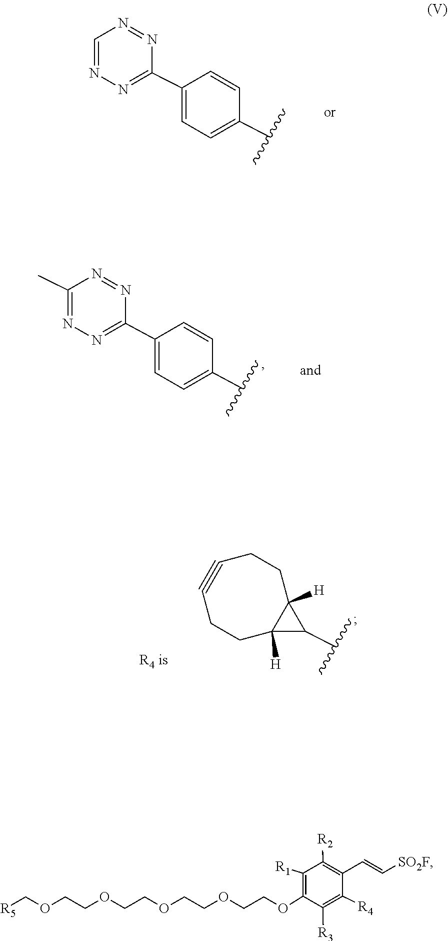
wherein
R₁, R₂ and R₄ are any substituent,
R₃ is H, an optionally substituted alkyl-F₃, an optionally substituted alkyl-N, or O—an optionally substituted alkyl, and
R₅ is —COOH, —NH₂, or 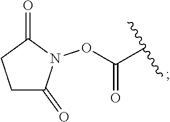 [SMILES: *C(=O)ON1C(=O)CCC1=O]
or

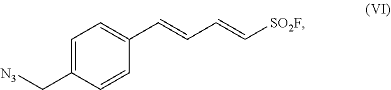 [SMILES: [N-]=[N+]=NCc1ccc(/C=C/C=C/S(=O)(=O)F)cc1]

96-97. (canceled)

98. The compound of claim 95 having the formula (V), optionally as the pharmaceutically acceptable salt.

99-103. (canceled)

104. The compound of claim 95, having the formula (VI), optionally as the pharmaceutically acceptable salt

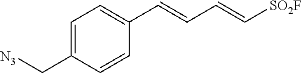 [SMILES: [N-]=[N+]=NCc1ccc(/C=C/C=C/S(=O)(=O)F)cc1]

105-107. (canceled)

108. A method for preventing and/or treating disease in a subject in need of the method comprising:
administering to the subject the conjugate of any of claim 1.

109-113. (canceled)

114. The compound of claim 95, having the formula (IV), optionally as the pharmaceutically acceptable salt.
